SEQUENCE LISTING (2 sequences)

SEQ ID NO: 1 - DNA, 23 bp, Artificial Sequence
  Primer hAluR
ttttttgagacggagtctcgctc

SEQ ID NO: 2 - DNA, 16 bp, Artificial Sequence
  Primer hAluF
ggcgcggtggctcacg

(19) United States
(12) Patent Application Publication
(10) Pub. No.: US 2023/0407264 A1
(43) Pub. Date: Dec. 21, 2023

(54) Hepatic Stem-Like Cells for the Treatment and/or the Prevention of Liver Disorders

(71) Applicants: Goliver Therapeutics, Nantes (FR); Nantes Universite, Nantes (FR); Institut National de la Santé et de la Recherche Médicale, Paris (FR)

(72) Inventors: Tuan Huy Nguyen, Nantes (FR); Angélique Fourrier, Sautron (FR); Raphaël Rispal, Oullins (FR); Frédéric Delbos, Saint-Sébastien-sur-Loire (FR)

(73) Assignees: Goliver Therapeutics, Nantes (FR); Nantes Universite, Nantes (FR); Institut National de la Santé et de la Recherche Médicale, Paris (FR)

(21) Appl. No.: 17/911,237

(22) PCT Filed: Mar. 15, 2021
(86) PCT No.: PCT/EP2021/056574

§ 371 Date: Sep. 13, 2022

(30) Foreign Application Priority Data
Mar. 13, 2020 (EP) 20305267.5

(51) Int. Cl. C12N 5/071; A61K 35/407; A61P 1/16

(52) U.S. Cl. CPC C12N 5/0672; C12N 5/0671; A61K 35/407; A61P 1/16

(57) ABSTRACT

The present invention relates to a population of cells comprising hepatic stem-like cells and therapeutic use thereof, for the treatment and the prevention of fulminant liver disorders. The hepatic stem-like cells according to the invention may be safely and reproducibly generated from pluripotent stem cells. In addition, although the hepatic stem-like cells according to the invention do not display the phenotype of physiologically mature hepatic cells, as they are lacking the albumin expression marker (ALB−), they may still be transplanted in a diseased liver with acute failure, rescue the diseased liver and promote liver regeneration. Moreover, various protocols of preparation of hepatic stem-like cells according to the invention may be implemented, all resulting in high quality and high yield of production. Finally, the hepatic stem-like cells according to the invention may be cryopreserved and may also be prepared as spheroid particles.

### SEQUENCE LISTING SUBMISSION VIA EFS-WEB

A computer readable text file, entitled “SequenceListing.txt” created on Sep. 6, 2022 with a file size of 823 bytes contains the sequence listing for this application and is hereby incorporated by reference in its entirety.

### FIELD OF INVENTION

The present invention relates to a population of cells comprising hepatic stem-like cells, and their therapeutic use for the treatment and/or the prevention of fulminant liver disorders. In particular, hepatic stem-like cells that are not expressing the ALB marker (ALB−) and that are expressing the AFP marker (AFP+), e.g. obtained from pluripotent stem cells (pSCs), may be injected in a liver having a fulminant liver disorder, such as an acute liver failure (ALF) or acute chronic liver failure (ACLF), and may promote liver regeneration.

### BACKGROUND OF INVENTION

Liver disorders affect millions of people worldwide. Among the liver disorders, fulminant liver disorders are characterized by a fast and severe dysfunction of the liver physiological performances and encompass disorders, such as, the acute liver failure (ALF) and the acute chronic liver failure (ACLF). The ACLF encompasses itself a liver disease characterized by an acute episode of liver failure, which is the consequence of progressive liver degradation associated with chronic liver diseases, such as, e.g., the non-alcoholic steato-hepatitis (NASH), alcoholic hepatitis, viral-induced hepatitis, cryptogenic liver diseases, malignant liver diseases such as hepatocellular carcinoma and cholangiocarcinoma, carcinoma, autoimmune hepatitis, vascular liver diseases such as Budd-Chiari syndrome, cholestatic liver diseases, inherited metabolic liver diseases such as Wilson's disease and urea cycle defects. In other words, a fulminant liver disorder refers to any disease prioritized for liver transplantation when using a scoring system for organ allocation such as the Model for End-stage Liver Disease (MELD) (Martin et al., 2014).

Liver transplantation is currently considered as the gold standard for treating patients with fulminant and/or severe metabolic liver disorders. Tens of thousands of patients are on the waiting lists for liver transplantation in the Western countries. From 1968 to 2015, approximately 130,000 liver transplantations were performed in Europe (European Liver Transplant Registry). However, 10 to 20% of patients are dying from not having received a transplant while on the waiting lists for liver transplantation, because of a shortage of organs' donors.

ALF and ACLF are short-term life-threatening diseases and rare conditions in which rapid deterioration of liver results in altered mentation and coagulopathy in individuals without (ALF) or with (ACLF) known pre-existing liver disorder. The main clinical signs of ALF and ACLF are rapid-onset jaundice of the skin and the eyeballs, pain in the abdomen, nausea, vomiting, weakness, and changes in mental status that can begin as mild confusion and progress to coma and to extra-hepatic multi-organ failures. The biochemical presentation of ALF and ACLF usually includes abnormal liver biochemical values and coagulopathy. In agreement with the clinical practice, and as mentioned above, the current treatment of choice for severe forms of ALF and ACLF is orthotopic liver (OLT).

However, OLT is severely limited due to the shortage of donors. To date, hepatocyte transplantation (HT) has become considered as an alternative to OLT and it has been found to improve liver functions in patients. HT has been demonstrated, and published worldwide showing the safety and preliminary efficacy of the technique (Dhawan et al.; 2010; Hansel et al.; 2014; Dhawan et al., 2019). However, obtaining large amounts of functional hepatocytes and with reproducible quality is difficult. In addition, patients receiving HT may also be treated with immunosuppressive agents, so as to limit transplant rejection.

There is thus a crucial need to explore the potential of new cell types, which include stem cells, to be amplified in vitro and subsequently differentiated into hepatocytes. Illustratively, definitive endoderm stem cells, human embryonic stem cells (hESCs), human induced pluripotent stem cells (hiPSCs) and multipotent stem cells, such as mesenchymal stem cells, have been used to generate hepatocytes or hepatocyte-like cells (HCLs) (Pareja et al., 2017).

For example, WO2016043666 disclosed methods of differentiating definitive endoderm stem cells in order to obtain hepatocyte-like cells (HLCs).

Indeed, human embryonic stem cells (hESCs) represent, in theory, an unlimited source of functional hepatocytes for liver regeneration. Human embryonic stem cells (hESCs) can be efficiently differentiated to hepatocytes-like cells (HLCs) in vitro, although intermediate cells may display an immature gene expression profile (see Cameron et al.; 2015). These HLCs share many of functions and gene expression profile with cells found in the adult liver (Payne et al.; 2011). Researchers could show in an acetaminophen-induced acute liver failure model in mice that the neonatal HLCs are able to repopulate and regenerate a diseased liver in vivo, without inducing a tumor. These data provide a convincing proof of concept that hESCs derived HLCs may be an alternative effective treatment to liver transplantation for treating liver diseases (Tolosa et al.; 2015).

In the same spirit, Roelandt et al. (2010) have attempted to provide the state in the art with hepatocytes derived from hESCs by the mean of differentiation culture media. Differentiation of definitive endoderm cells into hepatocytes necessitate the implementation of protocols involving a precise sequence of induction with various activators and/or inhibitors of physiological signalization pathways, as well as the presence of various growth factors. Hepatocyte-like cells are produces within 20 days, and are further characterized by their detoxifying capacities, as these cells are expressing markers that are proper to mature hepatocytes such as albumin and Cyp450s. Siller at al. (2015), further provided a method for differentiating pluripotent stem cells into hepatocytes in growth factor-free culture media.

Moreover, WO2019055345 disclosed methods for generating hepatocyte-like cells (HLCs) from human induced pluripotent stem cells (hiPSCs) for regenerative medicine. HLCs are capable of ammonium metabolism and possess detoxifying properties, as they express the albumin marker (ALB+) and may be used for treating patients with fulminant liver failure.

In addition, Takayama et al. (2013) provided the state of the art with hESCs-derived and hiPSCs-derived hepatoblast-like cells (HBCs). The authors showed that both early passaged HBCs (P0; ALB− cells) and late passage HBCs (P10; ALB+ cells) could be transplanted in the liver. The hepatocyte functionality of the hESC-derived HBC P0 or HBC P10 after transplantation was assessed by measuring secreted human ALB levels in the recipient mice. Takayama et al. observed that the ALB expression levels were higher upon transplantation with the HBC P10, as compared to HBC P0, which only resulted in a very weak ALB expression. Altogether, the results suggest to take advantage of HBC P10 (ALB+ cells) for liver transplantation, as the detoxifying properties associated with ALB are the most advanced. In agreement with these observations, Takayama et al. (2017) later published a study using later passaged HBCs (P10), which cells were specifically used as cells of choice for cell transplantation in liver therapy.

Human fetal liver cell transplantation has been also widely assessed for the treatment of liver diseases (see, e.g., Pietrosi et al., 2015; Rao et al., 2008; Zheng et al., 2006; Jochheim et al., 2004). Similarly to the hepatocytes disclosed by Roelandt et al., the human fetal liver cells disclosed by these studies are expressing the albumin protein (ALB+), which is a marker of mature hepatocytes.

Altogether, it emerges from the practice in the state of the art that the hepatic cells generated by these protocols are characterized by markers that are usually representative of a mature status of hepatic cells from a healthy liver, i.e. an expression of albumin (ALB+), which marker is associated with others properties of mature hepatocytes such as urea metabolism and CYP450 detoxifying properties, such as CYP2E1, CYP3A7 and some CYP3A4 activities (see Carpenter et al., 1996; Chinnici et al., 2015; Pietrosi et al., 2015).

Strikingly, although it is in theory feasible to transplant non mature hepatocytes, the consensus of having mature hepatocytes for transplantation is however well established in the scientific community, as illustrated by the following statement: “transplanted cells need to rescue liver functions promptly and therefore are required to be fully mature” (Goldman and Gouon-Evans, 2016). However, although highly reproducible, these protocols involve a long process, which is time consuming, and prone to viral and/or bacterial contamination.

Finally, cryopreservation of hepatocytes is a key in cell therapy for emergency transplantation in patients with ALF and ACLF. However, the difficulty with cryopreservation is due to cells being subjected to damaging conditions during both freezing and thawing steps leading to decreased cell viability (Terry et al.; 2010).

Therefore, there is a need to counteract the shortage of liver donors for liver transplantation and to provide means to easily generate hepatic cells that may be administered in a subject in need of a liver therapy, in particular, within limited amount of time, with high yield and of high quality. There is also a need to provide highly quantitative and qualitative cells that are compatible for regenerating and/or repairing a diseased liver, in particular in a liver undergoing a fulminant liver disorder, more particularly ALF or ACLF. There is further a need for providing a non-limited source of hepatic cells, irrespective of whether there is a shortage of liver donors, allowing both autologous and allogenic (heterologous) transplantation therapies. There is also a need for providing hepatic cells that resist to storage conditions, including cryopreservation. There is further a need to provide hepatic cells that could be administered without co-administration of immunosuppressive agents, as they would be universally transplantable. Finally, there is a need to provide new approaches to generate highly valuable therapeutic hepatic cells, in particular, approaches that are complying with the Good Manufacturing Practice (GMP).

### SUMMARY

A first aspect of the invention relates to a population of cells, in particular an isolated population of cells, comprising at least 5% of hepatic stem-like cells expressing the alpha-fœtoprotein marker (AFP+) and not expressing the albumin marker (ALB−), or an extract thereof.

In certain embodiments, the hepatic stem-like cells are further expressing the T-Box Transcription Factor 3 marker (TBX3+) and/or the Hepatocyte Nuclear Factor 4 Alpha marker (HNF4A+), preferably the T-Box Transcription Factor 3 marker (TBX3+) and the Hepatocyte Nuclear Factor 4 Alpha marker (HNF4A+).

In some embodiments, the hepatic stem-like cells are cryopreserved.

Another aspect of the invention relates to a particle, in particular a spheroid, comprising a population of cells comprising hepatic stem-like cells, or an extract thereof, according to the instant invention.

In one aspect, the invention relates to a suspension comprising a population of cells comprising hepatic stem-like cells, or an extract thereof, according to the instant invention.

A further aspect of the invention pertains to a pharmaceutical composition comprising (i) a population of cells comprising hepatic stem-like cells, or an extract thereof, and/or at least one particle, and/or a suspension, according to the instant invention, and (ii) a pharmaceutically acceptable vehicle.

A still further aspect of the invention relates to a medical device comprising a population of cells comprising hepatic stem-like cells, or an extract thereof, and/or at least one particle and/or a suspension, and/or a pharmaceutical composition, according to the instant invention.

In some aspect, the invention also relates to a non-human animal model comprising a heterologous population of cells comprising hepatic stem-like cells, or an extract thereof, according to the instant invention.

Another aspect of the invention relates to a hepatic stem-like cell, or an extract thereof, as defined in the instant disclosure, or the population of cells comprising hepatic stem-like cells, or an extract thereof, or the particle, or the suspension, or the pharmaceutical composition according to the instant invention, for use as a medicament.

A further aspect of the invention relates to a hepatic stem-like cell, or an extract thereof, as defined in the instant disclosure, or the population of cells comprising hepatic stem-like cells, or an extract thereof, or the particle, or the suspension, or the pharmaceutical composition, or the medical device according to the instant invention, for use in preventing and/or treating a fulminant liver disorder.

In one aspect, the invention relates to the hepatic stem-like cell, or an extract thereof, as defined in the instant disclosure, or the population of cells comprising hepatic stem-like cells, or an extract thereof, or the particle, or the suspension, or the pharmaceutical composition, or the medical device, for use according to the instant invention, wherein the fulminant liver disorder is an acute liver failure (ALF) or an acute chronic liver failure (ACLF).

A still further aspect of the invention relates to the hepatic stem-like cell, or an extract thereof, as defined in the instant invention, or the population of cells comprising hepatic stem-like cells, or an extract thereof, or the particle, or the suspension, or the pharmaceutical composition, or the medical device, for use according to the instant invention, and wherein the ACLF is associated with a liver disease selected in the group consisting of the non-alcoholic steatohepatitis (NASH); alcoholic hepatitis; viral-induced hepatitis; a cryptogenic liver disease; a malignant liver disease, such as hepatocellular carcinoma and cholangiocarcinoma; autoimmune hepatitis, a vascular liver disease, such as Budd-Chiari syndrome; a cholestatic liver disease; and an inherited metabolic liver disease, such as, Wilson's disease and an urea cycle disorder.

In one further aspect, the invention relates to the use of a cryopreserved population of cells comprising hepatic stem-like cells, or an extract thereof, according to the instant invention, for preparing a particle, as defined herein.

Another aspect of the invention relates to an in vitro method for screening a drug, said method comprising the steps of:
a) providing at least one hepatic stem-like cell, or an extract thereof, as defined herein, and/or the population of cells comprising hepatic stem-like cells, or an extract thereof, and/or particle, and/or suspension according to the instant invention;
b) contacting said at least one cell or an extract thereof, and/or said population of cells or extract thereof, and/or said particle, and/or said suspension, from step a), with a drug candidate;
c) measuring one or more biological parameter(s) and optionally comparing said one or more biological parameter(s) with one or more reference parameter(s);
d) determining whether the drug candidate is of therapeutic and/or diagnostic interest.

In one aspect, the invention relates to a kit for treating and/or preventing a fulminant liver disorder, said kit comprising:
a) hepatic stem-like cells, or an extract thereof, as defined herein, or a population of cells comprising hepatic stem-like cells, or an extract thereof, or particle, or suspension, or pharmaceutical composition according to the instant invention; and
b) a mean to administer said cells or extract thereof, population or extract thereof, or particle, or suspension or pharmaceutical composition.

### Definitions

In the present invention, the following terms have the following meanings:
“About” preceding a figure encompasses plus or minus 10%, or less, of the value of said figure. It is to be understood that the value to which the term “about” refers is itself also specifically, and preferably, disclosed.
“Comprise” is intended to mean “contain”, “encompass” and “include”. In some embodiments, the term “comprise” also encompasses the term “consist of”.
“Fulminant liver disorder” refers to a rapid and severe liver deterioration condition in an individual, with or without known pre-existing or diagnosed liver disease. It is to be understood that a fulminant liver disorder is itself a non-chronic disease, but may arise from a chronic liver condition. As used herein, a fulminant liver disorder refers to any liver disease prioritized for liver transplantation when using a scoring system for organ allocation such as the Model for End-stage Liver Disease (MELD) (Martin et al.; 2014). Examples of liver disease prioritized for liver transplantation include acute liver disorder (ALF) and acute chronic liver disorder (ACLF).
“Liver transplantation” refers to a surgical procedure performed to remove a diseased or injured liver and replace it with a whole or a portion of a healthy liver from another person (namely, the donor). The liver is the sole organ in the body able to self-regenerate, a transplanted segment of a liver can grow to normal size within weeks.
“Acute liver failure”, also termed “ALF”, refers to a highly specific and rare syndrome, characterized by an acute abnormality of liver blood tests in an individual without underlying chronic liver disease. ALF is mainly characterized by a mortality of 80% in absence of liver transplantation, as acknowledged in the publication of Larsen et al. (2016).
“Acute chronic liver failure”, also termed “ACLF”, refers to a highly specific and rare syndrome, characterized by an acute abnormality of liver blood tests in an individual with underlying chronic liver disease. In some embodiments, ACLF may also refer to as “acute-on-chronic liver failure”, as disclosed in Arroyo et al. (2016).
“Hepatic stem-like cells”, refers to, but is not limited to, auto-renewable hepatic stem cells which are capable of proliferating in suitable culturing conditions, and capable of differentiation into several cell types, in particular into hepatic cells (also referred to as hepatocytes) or cholangiocytes. The “hepatic stem-like cells” according to the invention are particularly characterized by the expression and/or non-expression of a set of specific markers. As used herein, the hepatic stem-like cells according to the invention, also referred to as “pStemHeps”, differ from mature hepatic cells in that they express the AFP marker (AFP+, a marker of immature hepatocytes) and do not express the ALB marker (ALB−). Indeed, the ALB marker is usually associated with the metabolic and detoxifying properties of mature hepatic cells.
“Cells derived from hepatic stem-like cells”, as used herein, refers to cells that are differentiated from hepatic stem-like cells into a differentiated cell type. Illustratively, cells derived from hepatic stem-like cells may encompass hepatic-like cells (HLCs) and cholangiocytes, and constitute the progeny of the hepatic stem-like cells according to the invention.
“Hepatic-like cells” or “HLCs”, as used herein, refers to hepatic cells that have been generated in vitro, and that possess the main markers of mature hepatocyte usually found within a functional healthy liver. In particular, HLCs possess (express) the markers associated with the detoxifying properties of a functional healthy liver, such as the ALB marker and the CYP3A4 marker.
“Expressing” or “expression” refers to the synthesis of a significant detectable level of a marker of interest, at the nucleic acid (RNA) level and/or the polypeptide or protein level. By extension, “expressing” or “expression” also refers to the level itself.
“Not expressing” or “non-expression” refers to the absence of synthesis of a significant detectable level of a marker of interest, at the nucleic acid (RNA) level and/or the polypeptide or protein level. By extension, “not expressing” or “non-expression” also refers to the level itself.
“Marker” refers to a molecule, preferably a protein, a glycoprotein or a lipoprotein that is expressed or not expressed, in particular differentially expressed or not expressed, by a given cell or a population of cells, and which expression level may be measured by suitable techniques (e.g., RT-PCR, RNA sequencing, ELISA, FACS, western blot, proteomics, immunofluorescence staining, protein activity), in order to characterize said cell or population of cells.
“Isolated” refers to a cell or population of cells that is removed from the initial culture medium that has allowed to generate this cell or population.
“Extract” refers to any cellular fraction, including cytosolic fraction, cytoplasmic fraction, membrane fractions, soluble fractions, insoluble fractions, vesicles, exosomes, and combination thereof; or to a culture supernatant obtained from a culture of hepatic stem-like cells, or a population, in particular an isolated population, of cells comprising hepatic stem-like cells according to the invention. In some embodiments, the extract comprises particles, such as extracellular vesicles (EVs), exosomes, or exosome-like particles.
“Exosome” refers to an extracellular nanovesicle that is naturally secreted/released from cells upon fusion of an intermediate endocytic compartment, the multivesicular body (MVB), with the plasma membrane. In other words, exosome is intended to relate to the intraluminal vesicle that is secreted/released into the extracellular milieu.
“Suspension” refers to a state in which the cells are cultured in 3D and floating in a culture medium.
“Particle/Spheroid” is meant to refer to a 3D particle wherein cells are aggregated to one another. The term “spheroid” is also intended to refer specifically to a particle with a spherical shape, whereas “particles” may have oval or tubular shapes.
“Extracorporeal bioartificial liver” or “EBAL” is meant to refer to an external medical device comprising cells with hepatic functions. Said device may be connected to a patient with a liver disorder, by the mean of the systemic circulation, and is intended to perform the detoxifying activity devoted to a functional healthy liver.
“Treating” or “treatment” or “alleviation” refers to both therapeutic treatment and prophylactic or preventative measures, wherein the object is to prevent or slow down (lessen) the targeted pathologic condition or disorder, in particular a liver disorder, more particularly a fulminant liver disorder. Those in need of treatment include those already with said disorder as well as those prone to develop the disorder or those in whom the disorder is to be prevented. An individual is successfully “treated” for a liver disorder, particularly a fulminant liver disorder, if, after receiving a therapeutic amount of hepatic stem-like cells according to the present invention, the individual shows observable and/or measurable reduction in or absence of one or more of the symptoms associated with the liver disorder (particularly the fulminant liver disorder); reduced morbidity and mortality, and improvement in quality of life issues. The above parameters for assessing successful treatment and improvement in the disease are readily measurable by routine procedures familiar to physician or authorized personnel.
“Preventing” refers to keeping from happening, and/or lowering the chance of the onset of, or at least one adverse effect or symptom of, a liver disease, disorder or condition associated with a deficiency in or absence of an organ, tissue or cell function, in particular of a fulminant liver disorder.
“Therapeutically efficient amount” refers to the level or the amount of the active agent that is aimed at, without causing significant negative or adverse side effects to the target, (1) delaying or preventing the onset of a liver disease, disorder, or condition, in particular of a fulminant liver disorder; (2) slowing down or stopping the progression, aggravation, or deterioration of one or more symptoms of a liver disease, disorder, or condition, in particular of a fulminant liver disorder; (3) bringing about ameliorations of the symptoms of a liver disease, disorder, or condition, in particular of a fulminant liver disorder; (4) reducing the severity or incidence of a liver disease, disorder, or condition, in particular of a fulminant liver disorder; or (5) curing a liver disease, disorder, or condition, in particular of a fulminant liver disorder. A therapeutically effective amount may be administered prior to the onset of a liver disease, disorder, or condition, in particular of a fulminant liver disorder, for a prophylactic or preventive action. Alternatively, or additionally, the therapeutically effective amount may be administered after the onset of a liver disease, disorder, or condition, in particular of a fulminant liver disorder, for a therapeutic action. In one embodiment, a therapeutically effective amount of the composition is an amount that is effective in reducing at least one symptom of a liver disease, disorder or condition, in particular of a fulminant liver disorder.
“Liver regeneration” refers to the ability of the liver to regain its functional biological or functional properties. “Liver regeneration” may encompass an increase of the proliferation of the endogenous “healthy” hepatocytes, or of endogenous stem cells and their subsequent differentiation into hepatocytes to compensate the dead of the “diseased” hepatocytes leading to an increase of the healthy liver tissue and healthy liver mass. “Liver regeneration” may also encompass a reduction of the liver inflammation associated with the liver disease.
“Pharmaceutically acceptable vehicle” refers to a vehicle that does not produce any adverse, allergic or other unwanted reactions when administered to an animal individual, preferably a human individual. It includes any and all solvents, dispersion media, coatings, antibacterial and antifungal agents, isotonic and absorption delaying agents and the like. For human administration, preparations should meet sterility, pyrogenicity, general safety, quality and purity standards as required by regulatory Offices, such as, e.g., the Food and Drug Administration (FDA) in the United States or the European Medicines Agency (EMA) in the European Union.
“Individual” is intended to refer to an animal individual, preferably a mammal individual, more preferably a human individual. Among the non-human mammal individuals of interest, one may non-limitatively mention pets, such as dogs, cats, guinea pigs; animals of economic importance such as cattle, sheep, goats, horses, monkeys. In one embodiment, an individual may be a “patient”, i.e. a warm-blooded animal, more preferably a human, who/which is awaiting the receipt of, or is receiving medical care or was/is/will be the object of a medical procedure, or is monitored for the development of a disease, disorder or condition, in particular, a liver disease, more particularly a fulminant liver disorder. In one embodiment, the individual is an adult (for example a human subject above the age of 18). In another embodiment, the individual is a child (for example a human subject below the age of 18). In one embodiment, the individual is a male. In another embodiment, the individual is a female.

### DETAILED DESCRIPTION

Unexpectedly, the inventors have shown that hepatic stem-like cells according to the invention, which have a markers' profile that does not comply with the markers' profile of mature hepatic cells, may be of therapeutic use for treating a fulminant liver disorder, in particular an acute liver failure (ALF) or an acute chronic liver failure (ACLF), which requires very fast regeneration of the liver, in particular regeneration of the healthy liver tissue within the diseased liver tissue. Contrarily to the prejudice in the state of the art, the population of cells comprising hepatic stem-like cells according to the invention, i.e. that express the AFP marker but do not express the ALB marker, which ALB marker is generally associated, together with the CYP3A4 marker, with the detoxifying function of mature hepatocytes, is still able to significantly reduce parameters such as the alanine aminotransferase (ALAT) concentration in the serum and the hepatic cell necrosis, very quickly, i.e. within 24 h after injection.

In other words, hepatic stem-like cells according to the invention, although they are not mature hepatic cells, are able to achieve regeneration of a diseased liver in an individual with fulminant liver failure, requiring very fast regeneration of the healthy liver tissue.

Without to be bound to a theory, the inventors consider that the presence of the detoxifying function usually found in mature hepatic cells, represented by the ALB and the CYP3A4 markers, is not necessary at the time of initiation of the treatment and that a stem cells-based cellular therapy relying upon a population of cells with less advanced differentiation status, is therapeutically satisfactory, contrarily to the prejudice in the field of liver transplantation.

In addition, the inventors have shown that non-limited amounts of hepatic stem-like cells may be generated from human pluripotent stem cells (hPSCs), and in particular from human embryonic stem cells (hESCs), undergoing numerous differentiation protocols.

Finally, the inventors have shown that transplantation of the hepatic stem-like cells according to the invention may be performed even in the absence of immunosuppressors, suggesting that the risk of acute transplant rejection is very low. These results therefore strongly suggest that allogenic transplantation of hepatic stem-like cells according to the invention may be safely performed in patients in need of the liver therapy, in particular in need of liver transplantation.

One aspect of the invention relates to a hepatic stem-like cell, in particular an isolated hepatic stem-like cell, expressing the alpha-fœtoprotein marker (AFP+) and not expressing the albumin marker (ALB−), or an extract thereof.

Within the scope of the invention, the alpha-fœtoprotein AFP also refers non-limitatively to the alpha-fetoprotein, alpha-1-fetoprotein, alpha fetoglobulin, HPAFP, AFPD and FETA. Within the scope of the invention, the albumin also refers non-limitatively to the serum albumin, PRO0883, PRO0903, PRO1341 and HSA.

In certain embodiments, the cell, in particular the human cell, is expressing the human alpha-fœtoprotein marker (AFP+) and not expressing the human albumin marker (ALB−), or an extract thereof.

The invention also refers to an in vitro cell culture of hepatic stem-like cells, expressing the alpha-fœtoprotein marker (AFP+), and not expressing the albumin marker (ALB−), or an extract thereof.

The invention also relies upon a population of cells comprising hepatic stem-like cells expressing the alpha-fœtoprotein marker (AFP+) and not expressing the albumin marker (ALB−), or an extract thereof.

More particularly, the invention relates to a population of cells, in particular an isolated population of cells, comprising hepatic stem-like cells expressing the alpha-fœtoprotein marker (AFP+) and not expressing the albumin marker (ALB−), or an extract thereof.

In some embodiments, the population of cells is a population of human cells.

Hence, another aspect of the invention relates to a population of cells, in particular an isolated population of cells, comprising at least 5% of hepatic stem-like cells expressing the alpha-fœtoprotein marker (AFP+) and not expressing the albumin marker (ALB−), or an extract thereof.

According to the instant invention, it is understood that a population of cells comprising hepatic stem-like cells according to the invention encompasses a population wherein at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or 100% of the cells are hepatic stem-like cells according to the invention.

Within the scope of the invention, the expression “at least about 5%” encompasses about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%.

In one embodiment, at least about 5% of the cells, preferably at least about 10% of the cells, are hepatic stem-like cells expressing the alpha-fœtoprotein marker (AFP+) and not expressing the albumin marker (ALB−).

In some embodiments, the population of cells is an in vitro population of cells, in particular an in vitro population of isolated cells.

The invention also refers to an in vitro cell culture comprising a population of cells, in particular a population of human cells, wherein at least about 5% of said cells are hepatic stem-like cells, particularly human hepatic stem-like cells, expressing the alpha-fœtoprotein marker (AFP+), particularly the human alpha-fœtoprotein marker (AFP+) and not expressing the albumin marker (ALB−), particularly the human albumin marker (ALB−), or an extract thereof.

In some embodiments, the hepatic stem-like cells are expressing the human alpha-fœtoprotein marker (AFP+) and not expressing the human albumin marker (ALB−).

In certain embodiments, the population is an isolated population of cells comprising hepatic stem-like cells expressing the alpha-fœtoprotein marker (AFP+), in particular the human alpha-fœtoprotein marker (AFP+) and not expressing the albumin marker (ALB−), in particular the human albumin marker (ALB−).

In some embodiments, the isolated population comprises at least about 50% of hepatic stem-like cells, preferably at least about 70% of hepatic stem-like cells according to the instant invention. In certain embodiments, the isolated population is a substantially pure population of hepatic stem-like cells according to the invention. Within the scope of the invention, the expression “substantially pure” is meant to refer to a population wherein said hepatic stem-like cells represent at least about 50% of the total cellular content of said population.

As used herein, the expression “at about least 50%” includes about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%.

In certain embodiments, the isolated population according to the invention comprises at least about 75%, preferably at least about 80%, preferably at least about 90% of hepatic stem-like cells according to the instant invention.

In practice, the population or the isolated population of cells comprising the hepatic stem-like cells according to the invention comprises hepatic stem-like cells and other cell types, such as e.g., modified fibroblast cells. In practice, the nature of the other cell types may depend of the nature of the cells used to generate the hepatic stem-like cells according to the invention.

Expression or absence of expression (non-expression) of these markers may be monitored by any suitable method known in the art, at the nucleic acid (mRNA) level or at the polypeptide or protein level. Illustratively, these methods may encompass at the nucleic acid level, a real-time RT-PCR (qPCR) analysis of RNA extracted from cultured cells with specific primers, RNA sequencing (RNASeq). At the polypeptide or protein level, these methods encompass an immunofluorescence analysis with markers-specific antibodies, Western blotting, ELISA, flow cytometry (also referred to as fluorescent activated cell sorting or FACS), or any functional protein activity assay.

It is understood that the percentage of hepatic stem-like cells comprised in the population of cells according to the invention may vary with respect to the method used to quantify the expression of selected markers, such as, in particular, the AFP and ALB markers, within said population of cells.

In some embodiments, the total relative expression of the cellular AFP mRNA may be measured by any suitable method known from the state of the art, or a method adapted therefrom. Illustratively, the total relative expression of the cellular AFP mRNA may be assessed by qPCR (quantitative PCR, also referred to as real-time PCR or RT-PCR). In practice, the total relative expression of the cellular AFP mRNA is assessed by the mean of the Taqman® technology, with the appropriate primers. In some embodiments, the relative expression of the cellular AFP mRNA may be normalized to a housekeeper gene expression, such as, e.g., GAPDH, and is expressed as fold of levels found in undifferentiated hESCs cells.

In certain embodiments, the total relative expression level of the cellular AFP mRNA is at least about 10² times higher than the expression level detected in AFP non expressing-cells when assessed by qPCR. As used herein, AFP non-expressing-cells are intended to refer to cells wherein significant detectable levels of AFP mRNA cannot be achieved when assessed by qPCR.

Within the scope of the instant invention, the term “at about least 10²” includes about 10², 5×10², 10³, 5×10³, 10⁴, 5×10⁴, 10⁵, 5×10⁵ 10⁶, 5×10⁶, 10⁷ or more. In some embodiments, the cells have a relative expression of AFP at least about 10³, preferably at least about 10⁵ times higher than the expression level detected in AFP non expressing-cells when assessed by qPCR.

In some embodiments, the immunofluorescence assay to measure the percentage of cells expressing the alpha-fœtoprotein marker (AFP+) and not expressing the albumin marker (ALB−) within the population of cells according to the invention may be performed by the mean of suitable anti-AFP and anti-ALB antibodies. Illustratively, suitable antibodies may be commercial antibodies, e.g., from Sigma Aldrich® (mouse anti-AFP antibody, A8452) and from Cedarlane® (mouse anti-ALB antibody, CL2513A). In practice, the immunofluorescence assay is performed according to the standard protocols from the state of the art, or protocols adapted therefrom. In some embodiments, the population of cells according to the invention, in particular the isolated population of cells comprises at least about 50% of hepatic stem-like cells according to the instant invention, as assessed by immunofluorescence.

In some embodiments, the flow cytometry assay to measure the percentage of cells expressing the alpha-fœtoprotein marker (AFP+) and not expressing the albumin marker (ALB−) within the population of cells according to the invention may be performed according to the standard protocols from the state of the art, or protocols adapted therefrom. In some embodiments, the population of cells according to the invention, in particular the isolated population of cells comprises at least about 5% of hepatic stem-like cells according to the instant invention, as assessed by flow cytometry.

In certain embodiments, the hepatic stem-like cells according to the invention secrete the expressed AFP.

In some embodiments, AFP secretion may be assessed by any suitable method known from the state of the art, or a method adapted therefrom. Illustratively, the AFP secretion may be assessed by the ELISA technique. In practice, the ELISA technique may be performed according to the standard protocols from the state of the art, or protocols adapted therefrom. In some embodiments, the ELISA technique is performed by the mean of a commercial kit, such as, e.g., the human AFP ELISA Quantification kit from ABCAM®. Assessment of the absence of ALB secretion in the cells' culture may be confirmed by ELISA, in particular by the mean of a commercial kit, such as, e.g., the Human Albumin ELISA Quantification kit from Bethyl®. When using commercial kits, the protocols are implemented following the manufacturer's instructions, with the appropriate controls. In some embodiments, hepatic stem-like cells secrete at least about 25 ng/10⁶ cells/24 h of the expressed AFP.

Within the scope of the instant invention, the term “at least about 25 ng/10⁶ cells/24 h of the expressed AFP” includes 25 ng, 50 ng, 100 ng, 200 ng, 300 ng, 400 ng, 500 ng, 600 ng, 700 ng, 800 ng, 900 ng, 1 μg, 2 μg, 3 μg, 4 μg, 5 μg/10⁶ cells/24 h or more. In some embodiments, the cells secrete at least 500 ng/10⁶ cells/24 h, preferably at least 1 μg/10⁶ cells/24 h of the expressed AFP. In some embodiments, the hepatic stem-like cells secrete from about 0.1 μg/10⁶ cells/24 h to about 100 μg/10⁶ cells/24 h of the expressed AFP, preferably from about 1 μg/10⁶ cells/24 h to about 70 μg/10⁶ cells/24 h of the expressed AFP.

In certain embodiments, the stem-like cells according to the invention further express the T-Box Transcription Factor 3 marker (TBX3+) and/or the Hepatocyte Nuclear Factor 4 Alpha marker (HNF4A+), preferably the T-Box Transcription Factor 3 marker (TBX3+) and the Hepatocyte Nuclear Factor 4 Alpha marker (HNF4A+).

In certain embodiments, the stem-like cells according to the invention further express the human T-Box Transcription Factor 3 marker (TBX3+) and/or the human Hepatocyte Nuclear Factor 4 Alpha marker (HNF4A+), preferably the human T-Box Transcription Factor 3 marker (TBX3+) and the human Hepatocyte Nuclear Factor 4 Alpha marker (HNF4A+).

In certain embodiments, the stem-like cells according to the invention further express at least one marker, in particular at least two markers, more particularly three markers, preferably human markers, selected in the group consisting of KRT19, EPCAM and TTR. In some embodiments, the stem-like cells according to the invention further express at least one marker, in particular at least two markers, more particularly three markers, even more particularly four markers, preferably human markers, selected in the group consisting of KRT19, EPCAM, TTR and HGF.

In some embodiments, the stem-like cells according to the invention further express at least one marker, in particular at least two markers, more particularly three markers, preferably human markers, selected in the group consisting of KRT19, EPCAM and TTR; and/or further express HGF, preferably human HGF.

In certain embodiments, the stem-like cells according to the invention express the alpha-fœtoprotein marker (AFP+), in particular the human alpha-fœtoprotein marker (AFP+) and not express the albumin marker (ALB−), in particular the human albumin marker (ALB−); and further express the T-Box Transcription Factor 3 marker (TBX3+) (particularly human TBX3+) and/or the Hepatocyte Nuclear Factor 4 Alpha marker (HNF4A+) (particularly human HNF4A+), preferably the T-Box Transcription Factor 3 marker (TBX3+) and the Hepatocyte Nuclear Factor 4 Alpha marker (HNF4A+) (particularly human TBX3+ and HNF4A+); and further express at least one marker, in particular at least two markers, more particularly three markers, preferably human markers, selected in the group consisting of KRT19, EPCAM and TTR; and/or further express HGF, preferably human HGF.

In some embodiments, the hepatic stem-like cells according to the invention express the AFP, TBX3 and HNF4A markers, and do not express the ALB marker. In certain embodiments, the hepatic stem-like cells according to the invention express the AFP, TBX3, HNF4A and HGF markers, and do not express the ALB marker. In some embodiments, the hepatic stem-like cells according to the invention express the AFP, TBX3, HNF4A and TTR markers, and do not express the ALB marker. In certain embodiments, the hepatic stem-like cells according to the invention express the AFP, TBX3, HNF4A, HGF and TTR markers, and do not express the ALB marker. In some embodiments, the hepatic stem-like cells according to the invention express the AFP, TBX3, HNF4A and EPCAM markers, and do not express the ALB marker. In certain embodiments, the hepatic stem-like cells according to the invention express the AFP, TBX3, HNF4A, EPCAM and HGF markers, and do not express the ALB marker. In some embodiments, the hepatic stem-like cells according to the invention express the AFP, TBX3, HNF4A, EPCAM, TTR and KRT19 markers, and do not express the ALB marker.

In certain embodiments, the hepatic stem-like cells according to the invention express the AFP, TBX3, HNF4A, EPCAM, TTR, KRT19, and HGF markers, and do not express the ALB marker. Preferably the marker is a human marker.

In some embodiments, said hepatic stem-like cells according to the invention are further expressing the EPCAM marker (EPCAM+), and/or are not expressing the CYP3A4 cytochrome marker (CYP3A4−).

In certain embodiments, the hepatic stem-like cells according to the invention are expressing the human EPCAM marker (EPCAM+), and/or are not expressing the human CYP3A4 cytochrome marker (CYP3A4−).

In some embodiments, the stem-like cells according to the invention further express the SOX17 marker (SOX17+), preferably the human SOX17 marker and/or the APOA1 marker (APOA1+), preferably the human APOA1 marker.

In some embodiments, the stem-like cells according to the invention further express the SERPINA1 marker (SERPINA1+), preferably the human SERPINA1 marker.

In certain embodiments, the stem-like cells according to the invention express the alpha-fœtoprotein marker (AFP+), in particular the human alpha-fœtoprotein marker (AFP+) and not express the albumin marker (ALB−), in particular the human albumin marker (ALB−); and further express the T-Box Transcription Factor 3 marker (TBX3+) (particularly human TBX3+) and/or the Hepatocyte Nuclear Factor 4 Alpha marker (HNF4A+) (particularly human HNF4A+), preferably the T-Box Transcription Factor 3 marker (TBX3+) and the Hepatocyte Nuclear Factor 4 Alpha marker (HNF4A+) (particularly human TBX3+ and HNF4A+); and further express at least one marker, in particular at least two markers, more particularly three markers, preferably human markers, selected in the group consisting of KRT19, EPCAM and TTR; and/or further express HGF, preferably human HGF; and/or further express the SOX17 marker (SOX17+), preferably the human SOX17 marker and/or the APOA1 marker (APOA1+), preferably the human APOA1 marker; and/or further express the SERPINA1 marker (SERPINA1+), preferably the human SERPINA1 marker.

In one embodiment, the hepatic stem-like cells, in particular within the population of cells, do not express the ALB marker (ALB−) and do not express the CYP3A4 marker (CYP3A4−), but in particular express the AFP marker (AFP+). It is understood that both the ALB and the CYP3A4 proteins are known to participate in the metabolic and detoxifying properties of a healthy adult liver. Therefore, in said embodiment, the hepatic stem-like cells within the population of cells does not possess the metabolic and detoxifying properties associated with the ALB and CYP3A4 markers.

In certain embodiments, the hepatic stem-like cells, in particular within the population of cells, may further be characterized by a combination of at least two of the following features:
an expression of one or more of the following markers, preferably human markers: APOA1, APOA2, APOA4, APOB, APOC3, APOE, BMP2, BMP4, CD164, CD24, CXCR4, DLKT, DPP4, FOXA2, GATA4, GATA6, GJA1, GSTA1, GSTA2, HNF1B, HNF4A, KI67, KRT18, KRT19, KRT8, SEPP1, SMAD7, SOD1, SPARC, TBX3, TTR, VIM, VTN;
an absence of expression (non-expression) of one or more of the following markers, preferably human markers: ABCB11, ASGR1, CYP1A2, CYP2A6, CYP2B6, CYP2B7P, CYP2C9, CYP2E1, CYP3A7, F9, NAGS, PDX1, UGT1A1.

In some embodiments, the hepatic stem-like cells according to the invention, in particular within the population of cells, may further be characterized by a combination of at least two of the following features:
an expression of one or more of markers, preferably human markers, selected in the group consisting of APOA1, APOA2, APOA4, APOB, APOC3, APOE, BMP2, BMP4, CD164, CD24, CD99, CXCR4, DCN, DLK1, DPP4, EPCAM, FGF19, FOXA2, GATA4, GATA6, GJA1, GPC3, GSTA1, GSTA2, HGF, HMOX1, HNF1B, HNF4A, IGF1, IGFBP3, IL6ST, ITGA6, KI67, KRT18, KRT19, KRT8, LCP1, MKI67, MYDGF, NODAL, PITX2, PROX1, SEPP1, SERPINA1, SMAD7, SNAI2, SOD1, SOX17, SPARC, TBX3, TTR, UGT3A1, VIM, and VTN; and/or
an absence of expression (non-expression) of one or more of markers, preferably human markers, selected in the group consisting of ABCB11, ASGR1, CYP1A2, CYP2A6, CYP2B6, CYP2B7P, CYP2C9, CYP2E1, CYP3A4, CYP3A7, F9, NAGS, PDX1, UGT1A1.

Within the scope of the invention, EPCAM also refers non-limitatively to the Epithelial Cell Adhesion Molecule, Tumor-Associated Calcium Signal Transducer 1, Major Gastrointestinal Tumor-Associated Protein GA733-2, Trophoblast Cell Surface Antigen 1, Adenocarcinoma-Associated Antigen, Cell Surface Glycoprotein Trop-1, Epithelial Glycoprotein 314, TACSTD1, EGP314, MIC18, TROP1, M4S1, KSA, Antigen Identified By Monoclonal Antibody AUA1, Human Epithelial Glycoprotein-2, Epithelial Cell Surface Antigen, Epithelial Glycoprotein, KS 1/4 Antigen, CD326 Antigen, GA733-2, HEGP314, HNPCC8, Ep-CAM, DIAR5, EGP-2, EGP40, KS1/4, MK-1, M1S2, ESA and EGP.

Within the scope of the invention, CYP3A4 also refers non-limitatively to the Cytochrome P450 Family 3 Subfamily A Member 4, Cytochrome P450 Subfamily IIIA Polypeptide 4, Cytochrome P450 Family 3 Subfamily A Polypeptide 4, Albendazole Monooxygenase (Sulfoxide-Forming), Taurochenodeoxycholate 6-Alpha-Hydroxylase, 1,8-Cineole 2-Exo-Monooxygenase, Cholesterol 25-Hydroxylase, Albendazole Sulfoxidase, Quinine 3-Monooxygenase, Cytochrome P450 NF-25, Cytochrome P450-PCN1, Cytochrome P450 3A3, Cytochrome P450 3A4, Cytochrome P450 HLp, Nifedipine Oxidase, CYPIIIA3, CYPIIIA4, CYP3A3, Cytochrome P450, Subfamily IIIA Polypeptide 3, Glucocorticoid-Inducible P450, P450-III, Steroid Inducible, Albendazole Monooxygenase, P450PCN1, P450C3, CYP3A, NF-25, CP33, CP34 and HLP.

Within the scope of the invention, APOA1 also refers non-limitatively to the Apolipoprotein A1, Apolipoprotein A-I, Apo-AI, Epididymis Secretory Sperm Binding Protein, Apo(A), ApoA-I; APOA2 also refers non-limitatively to the Apolipoprotein A2, Apolipoprotein A-II, Apo-AII, ApoA-II and ApoAII; APOA4 also refers non-limitatively to the Apolipoprotein A4, Apolipoprotein A-IV, Apo-AIV, and ApoA-IV; APOB also refers non-limitatively to the Apolipoprotein B, Apolipoprotein B-100, Apolipoprotein B48, Apo B-100, ApoB-100, ApoB-48, LDLCQ4, FCHL2 and FLDB; APOC3 also refers non-limitatively to the Apolipoprotein C3, Apolipoprotein C-III, Apo-CIII, ApoC-III and APOCIII; APOE also refers non-limitatively to the Apolipoprotein E, Alzheimer Disease 2 (APOE*E4-Associated, Late Onset), Apolipoprotein E3, LDLCQ5, APO-E, ApoE4, Apo-E, LPG and AD2; BMP2 also refers non-limitatively to the Bone Morphogenetic Protein 2, Bone Morphogenetic Protein 2A, BMP2A, BMP-2A, SSFSC, BMP-2 and BDA2; BMP4 also refers non-limitatively to the Bone Morphogenetic Protein 4, Bone Morphogenetic Protein 2B, BMP2B, BMP2B1, MCOPS6, BMP-2B, OFC11, BMP-4, ZYME and DVR4; CD164 also refers non-limitatively to the CD164 Molecule, Multi-Glycosylated Core Protein 24, Sialomucin Core Protein 24, CD164 Antigen, Sialomucin, Endolyn, MGC-24v, MGC-24, MUC-24, Deafness Autosomal Dominant 66, CD164 Antigen, DFNA66; CD24 also refers non-limitatively to the CD24 Molecule, CD24 Antigen, Signal Transducer CD24, CD24A, Small Cell Lung Carcinoma Cluster 4 Antigen, CD24 Antigen; CD99 also refers non-limitatively to the CD99 antigen, Antigen Identified By Monoclonal Antibodies 12E7, F21 And 013, T-cell surface glycoprotein E2, E2 antigen, MIC2X, MIC2Y, MIC2, cell surface antigen HBA-71, cell surface antigen 12E7, cell surface antigen MIC2, HBA71, MSK5X, 12E7; CXCR4 also refers non-limitatively to the C-X-C Motif Chemokine Receptor 4, Leukocyte-Derived Seven Transmembrane Domain Receptor, Lipopolysaccharide-Associated Protein 3, Stromal Cell-Derived Factor 1 Receptor, Chemokine (C-X-C Motif) Receptor 4, C-X-C Chemokine Receptor Type 4, LPS-Associated Protein 3, SDF-1 Receptor, CD184 Antigen, Fusin, LAP-3, LESTR, NPYRL, FB22, HM89, LCR1, Seven-Transmembrane-Segment Receptor Spleen, Chemokine (C-X-C Motif) Receptor 4, Seven Transmembrane Helix Receptor, Neuropeptide Y Receptor Y3, Neuropeptide Y3 Receptor, Chemokine Receptor, D2S201E, HSY3RR, NPYY3R, CXC-R4, CXCR-4, CD184, NPY3R, WHIMS, LAP3, NPYR, WHIM 3; DCN also refers non-limitatively to the Decorin, SLRR1B, Bone Proteoglycan II, DSPG2, PG40, Dermatan Sulphate Proteoglycans II, Small Leucine-Rich Protein 1B, Proteoglycan Core Protein, Decorin Proteoglycan, PG-S2, CSCD, PGII, PGS2, DCN 5; DLK1 also refers non-limitatively to the Delta Like Non-Canonical Notch Ligand 1, Protein Delta Homolog 1, DLK-1, DLK, PG2, Delta-Like 1 Homolog, Delta-Like Homolog, Preadipocyte Factor 1, Delta-Like 1 Homolog, Fetal Antigen 1, Secredeltin, Delta1, Pref-1, PREF1, FA1 and ZOG; DPP4 also refers non-limitatively to the Dipeptidyl Peptidase 4, Adenosine Deaminase Complexing Protein 2, Dipeptidylpeptidase IV, CD26 Adenosine Deaminase Complexing Protein 2, T-Cell Activation Antigen CD26, Dipeptidyl Peptidase IV, EC 3.4.14.5, ADCP-2, DPP IV, ADCP2, ADABP, TP103, CD26, Dipeptidyl-Peptidase 4, Dipeptidylpeptidase 4, CD26 Antigen and DPPIV; FGF19 also refers non-limitatively to the Fibroblast Growth Factor 19; FOXA2 also refers non-limitatively to the Forkhead Box A2, Hepatocyte Nuclear Factor 3-Beta, Forkhead Box Protein A2, Transcription Factor 3B, HNF-3-Beta, HNF-3B, TCF-3B and Hepatic Nuclear Factor-3-Beta; GATA4 also refers non-limitatively to the GATA Binding Protein 4, Transcription Factor GATA-4, GATA-Binding Factor 4, GATA-Binding Protein 4, TACHD, ASD2, VSD1 and TOF; GATA6 also refers non-limitatively to the GATA Binding Protein 6, Transcription Factor GATA-6, GATA-Binding Factor 6 and GATA-Binding Protein 6; GJA1 also refers non-limitatively to the Gap Junction Protein Alpha 1, Gap Junction 43 KDa Heart Protein, Gap Junction Alpha-1 Protein, Connexin-43, GJAL, Oculodentodigital Dysplasia (Syndactyly Type III), Gap Junction Protein Alpha-Like, Connexin 43, AVSD3, EKVP3, HLHS1, PPKCA, CMDR, CX43, EKVP, ODDD, Cx43 and HSS; GPC3 also refers non-limitatively to the Glypican 3, Intestinal Protein OCI-5, Glypican Proteoglycan 3, Glypican-3, GTR2-2, OCI-5, SGBS1, DGSX, MXR7, SGBS, SGB, Heparan Sulphate Proteoglycan, Secreted Glypican-3, SDYS, OCIS; GSTA1 also refers non-limitatively to the Glutathione S-Transferase Alpha 1, Glutathione S-Transferase A1, 13-Hydroperoxyoctadecadienoate Peroxidase, Androst-5-Ene-3,17-Dione Isomerase, GST Class-Alpha Member 1, GST HA Subunit 1, GST-Epsilon, EC 2.5.1.18, GSTA1-1, GTH1, Glutathione S-Transferase Ha Subunit 1, S-(Hydroxyalkyl)Glutathione Lyase A1, Glutathione S-Alkyltransferase A1, Glutathione S-Aryltransferase A1, Testicular Tissue Protein Li 80, Glutathione S-Transferase 2, EC 1.11.1., EC 5.3.3. and GST2; GSTA2 also refers non-limitatively to the Glutathione S-Transferase Alpha 2, Glutathione S-Transferase A2, GST Class-Alpha Member 2, GST HA Subunit 2, EC 2.5.1.18, GST-Gamma, GSTA2-2, GST2, GTH2, Testis Tissue Sperm-Binding Protein Li 59n, S-(Hydroxyalkyl)Glutathione Lyase A2, Glutathione S-Aralkyltransferase A2, Glutathione S-Alkyltransferase A2, Glutathione S-Aryltransferase A2, Liver GST2 and GTA2; HGF also refers non-limitatively to the Hepatocyte Growth Factor, HPTA, SF, Hepatocyte Growth Factor (Hepapoietin A; Scatter Factor), Fibroblast-Derived Tumor Cytotoxic Factor, Lung Fibroblast-Derived Mitogen, Hepatopoietin-A, Scatter Factor, F-TCF, HGFB, DFNB39; HMOX1 also refers non-limitatively to the Heme Oxygenase 1, HO-1, Heme Oxygenase (Decycling) 1, BK286B10, Heat Shock Protein 32-KD, HMOX1D, HSP32, HO1, HO; HNF1B also refers non-limitatively to the HNF1 Homeobox B, Hepatocyte Nuclear Factor 1-Beta, Homeoprotein LFB3, HNF-1-Beta, HNF-1B, VHNF1, TCF-2, TCF2, Variant Hepatic Nuclear Factor, Variant Hepatic Nuclear Factor 1, Transcription Factor 2 Hepatic, Transcription Factor 2, HNF1 Beta A, HNF1beta, HPC11, LF-B3, MODY5, FJHN, HNF2 and LFB3; HNF4A also refers non-limitatively to the Hepatocyte Nuclear Factor 4 Alpha, Nuclear Receptor Subfamily 2 Group A Member 1, Hepatocyte Nuclear Factor 4-Alpha, Transcription Factor HNF-4, Transcription Factor 14, TCF-14, TCF14, NR2A1, HNF4, Hepatic Nuclear Factor 4 Alpha, HNF4alpha10/11/12, HNF-4-Alpha, HNF4alpha, HNF4a7, HNF4a8, HNF4a9, NR2A21, FRTS4, MODY1, MODY and TCF3; IGF1 also refers non-limitatively to the Insulin Like Growth Factor 1, IGF-I, Mechano Growth Factor, Somatomedin-C, IGFI, IGF, MGF, Insulin-Like Growth Factor IB, IGF1A, IBP1; IGFBP3 also refers non-limitatively to the Insulin Like Growth Factor Binding Protein 3, IGF-Binding Protein 3, IBP3, Acid Stable Subunit Of The 140 K IGF Complex, Growth Hormone-Dependent Binding Protein, Binding Protein 53, Binding Protein 29, IGFBP-3, BP-53, IBP-3; IL6ST also refers non-limitatively to the Interleukin 6 Signal Transducer, Interleukin-6 Receptor Subunit Beta, Oncostatin-M Receptor Subunit Alpha, Gp130 Oncostatin M Receptor, IL-6 Receptor Subunit Beta, Membrane Glycoprotein 130, IL-6R Subunit Beta, CD130 Antigen, IL-6RB, SGP130, CD130, GP130, Gp130 Of The Rheumatoid Arthritis Antigenic Peptide-Bearing Soluble Form, Interleukin Receptor Beta Chain, Interleukin-6 Signal Transducer, Membrane Glycoprotein Gp130, IL-6R-Beta, CDW130, HIES4; ITGA6 also refers non-limitatively to the Integrin Subunit Alpha 6, CD49 Antigen-Like Family Member F, Integrin Alpha 6, CD49f, VLA-6, Integrin Alpha6B, CD49f Antigen, ITGA6B; KRT18 also refers non-limitatively to the Keratin 18, Cell Proliferation-Inducing Gene 46 Protein, Keratin, Type I Cytoskeletal 18, Keratin 18, Type I, CK-18, CYK18, K18, Cytokeratin 18, Cytokeratin-18 and Keratin-18; KRT19 also refers non-limitatively to the Keratin 19, Keratin Type I Cytoskeletal 19, 40-KDa Keratin Intermediate Filament, Keratin Type I 40-Kd, Keratin 19 Type I, Cytokeratin 19, CK-19, K19, Cytokeratin-19, Keratin-19, CK19 and K1CS; KRT8 also refers non-limitatively to the Keratin 8, Keratin Type II Cytoskeletal 8, Type-II Keratin Kb8, Keratin 8 Type II, Cytokeratin-8, CK-8, CYK8, K8, Keratin-8, CARD2, K2C8, CK8 and KO; LCP1 so refers non-limitatively to the Lymphocyte Cytosolic Protein 1, LC64P, PLS2, L-PLASTIN, Plastin-2, LCP-1, CP64, L-Plastin (Lymphocyte Cytosolic Protein 1) (LCP-1) (LC64P), BA139H14.1 (Lymphocyte Cytosolic Protein 1 (L-Plastin)), Lymphocyte Cytosolic Protein 1 (L-Plastin), Lymphocyte Cytosolic Protein-1 (Plasmin), Epididymis Secretory Protein Li 37, Plastin 2, L-Plastin, HEL-S-37, LPL 3; MKI67 also refers non-limitatively to the Marker Of Proliferation Ki-67, Antigen Identified By Monoclonal Antibody K1-67, Protein Phosphatase 1 Regulatory Subunit 10⁵, Proliferation Marker Protein Ki-67, Antigen Ki67, Proliferation-Related Ki-67 Antigen, Antigen KI-67, PPP1R105, MIB-1, MIB and KIA; MYDGF also refers non-limitatively to the Myeloid Derived Growth Factor, Interleukin 27 Working Designation, C19orf10, R33729_1, IL25, SF20, Stromal Cell-Derived Growth Factor SF20, Chromosome 19 Open Reading Frame 10, UPF0556 Protein C19orf10, EUROIMAGE1875335, Interleukin-25, IL27w, and IL27. NODAL also refers non-limitatively to the Nodal Growth Differentiation Factor, Nodal Homolog, Nodal Mouse Homolog, HTX5; PITX2 also refers non-limitatively to the Paired Like Homeodomain 2, Paired-Like Homeodomain Transcription Factor 2, ARP1, ALL1-Responsive Protein ARP1, Homeobox Protein PITX2, Pituitary Homeobox 2, Solurshin, Otlx2, RIEG1, Brx1, IGDS, RIEG, RGS, RS, Rieg Bicoid-Related Homeobox Transcription Factor 1, RIEG Bicoid-Related Homeobox Transcription Factor, All1-Responsive Gene 1, ASGD4, IGDS2, IRID2, IDG2, IHG2; PROX1 also refers non-limitatively to the Prospero Homeobox 1, Homeobox Prospero-Like Protein PROX1, Prospero-Related Homeobox 1; SEPP1 also refers non-limitatively to the Selenoprotein P, Selenoprotein P Plasma 1, SELP, SeP and SEPP; SERPINA1 also refers non-limitatively to the Serpin Family A Member 1, Alpha-1-Antitrypsin, AAT, Serpin Peptidase Inhibitor Clade A (Alpha-1 Antiproteinase, Antitrypsin) Member 1, Protease Inhibitor 1 (Anti-Elastase) Alpha-1-Antitrypsin, Alpha-1 Protease Inhibitor, Alpha-1-Antiproteinase, Serpin A1, Alpha1AT, A1AT, A1A, PI1, PI, Serine (Or Cysteine) Proteinase Inhibitor Clade A (Alpha-1 Antiproteinase, Antitrypsin) Member 1, Serpin Peptidase Inhibitor Clade A (Alpha-lantiproteinase, Antitrypsin) Member 1, Alpha-1-Antitrypsin Short Transcript Variant 1C4, Alpha-1-Antitrypsin Short Transcript Variant 1C5, Serpin Peptidase Inhibitor Clade A Member 1, Epididymis Secretory Sperm Binding Protein, Alpha-1-Antitrypsin Null, Alpha-1 Antitrypsin, PRO2275, NNIF; SMAD7 also refers non-limitatively to the SMAD Family Member 7, Mothers Against Decapentaplegic Homolog 7, Mothers Against DPP Homolog 8, MAD Homolog 8, HSMAD7, MADH7, MADH8, MAD, Mothers Against DPP Homolog 7, MAD Homolog 7, SMAD 7, CRCS3 and Smad7; SNAI2 also refers non-limitatively to the Snail Family Transcriptional Repressor 2, SLUGH, Snail Family Zinc Finger 2, Zinc Finger Protein SNAI2, Protein Snail Homolog 2, SLUGH1, SNAIL2, SLUG, Slug Homolog Zinc Finger Protein (Chicken), Slug (Chicken Homolog) Zinc Finger Protein, Neural Crest Transcription Factor SLUG, Neural Crest Transcription Factor Slug, Snail Homolog 2 (Drosophila), Snail Homolog, WS2D 3; SOD1 also refers non-limitatively to the Superoxide Dismutase 1, Superoxide Dismutase 1 Soluble, Superoxide Dismutase [Cu—Zn], EC 1.15.1.1, HSod1, Amyotrophic Lateral Sclerosis 1 (Adult), Epididymis Secretory Protein Li 44, Superoxide Dismutase Cystolic, Cu/Zn Superoxide Dismutase, Indophenoloxidase A, SOD Soluble, Homodimer, HEL-S-44, IPOA, ALS1, SOD and ALS; SOX17 also refers non-limitatively to the SRY-Box Transcription Factor 17, SRY (Sex Determining Region Y)-Box 17, Transcription Factor SOX-17, SRY-Box 17, SRY-Related HMG-Box Transcription Factor SOX17, and VUR3; SPARC also refers non-limitatively to the Secreted Protein Acidic And Cysteine Rich, Secreted Protein Acidic And Rich In Cysteine, Basement-Membrane Protein 40, Osteonectin, BM-40, ON, Secreted Protein Acidic Cysteine-Rich, Cysteine-Rich Protein and 0117; TBX3 also refers non-limitatively to the T-Box Transcription Factor 3, T-Box 3, T-Box Transcription Factor TBX3, T-Box Protein 3, Bladder Cancer Related Protein XHL, Ulnar Mammary Syndrome, TBX3-ISO, XHL and UMS; TTR also refers non-limitatively to the Transthyretin, Prealbumin Amyloidosis Type I, PALB, ATTR, TBPA, Epididymis Luminal Protein 111, Thyroxine-Binding Prealbumin, Carpal Tunnel Syndrome 1, Prealbumin, HsT2651, HEL111, CTS1 and CTS; UGT3A1 also refers non-limitatively to the UDP Glycosyltransferase Family 3 Member A1, UDP Glycosyltransferase 3 Family Polypeptide A1, UDP-Glucuronosyltransferase 3A1, UDPGT 3A1, FLJ34658; VIM also refers non-limitatively to the Vimentin and Epididymis Secretory Sperm Binding Protein; VTN also refers non-limitatively to the Vitronectin, Serum Spreading Factor, Complement S-Protein, Somatomedin B, S-Protein, V75, VN, Serum-Spreading Factor, Epibolin and VNT.

Within the scope of the invention, ABCB11 also refers non-limitatively to the ATP Binding Cassette Subfamily B Member 11, Bile Salt Export Pump, ATP-Binding Cassette Sub-Family B (MDR/TAP) Member 11, Progressive Familial Intrahepatic Cholestasis 2, ATP-Binding Cassette Sub-Family B Member 11, ABC Member 16 MDR/TAP Subfamily, BSEP, Sister P-Glycoprotein, EC 3.6.3.44, EC 7.6.2., EC 3.6.3, PFIC-2, ABC16, BRIC2, PFIC2, PGY4 and SPGP; ASGR1 also refers non-limitatively to the Asialoglycoprotein Receptor 1, C-Type Lectin Domain Family 4 Member H1, Hepatic Lectin H1, CLEC4H1, HL-1, ASGP-R 1, ASGPR 1, ASGPR1 and ASGPR; CYP1A2 also refers non-limitatively to the Cytochrome P450 Family 1 Subfamily A Member 2, Cytochrome P450, Subfamily I (Aromatic Compound-Inducible) Polypeptide 2, Cytochrome P450 Family 1 Subfamily A Polypeptide 2, Hydroperoxy Icosatetraenoate Dehydratase, Cholesterol 25-Hydroxylase, Cytochrome P450 1A2, Cytochrome P(3)450, Cytochrome P450-P3, Cytochrome P450 4, CYPIA2, Flavoprotein-Linked Monooxygenase, Aryl Hydrocarbon Hydroxylase, Microsomal Monooxygenase, Xenobiotic Monooxygenase, Dioxin-Inducible P3-450, EC 1.14.14.1, EC 1.14.14., EC 4.2.1.152, P450 Form 4, P450(PA), P3-450 and CP12; CYP2A6 also refers non-limitatively to the Cytochrome P450 Family 2 Subfamily A Member 6, Cytochrome P450 Subfamily IIA (Phenobarbital-Inducible) Polypeptide 6, Cytochrome P450 Family 2 Subfamily A Polypeptide 6, 1,4-Cineole 2-Exo-Monooxygenase, Coumarin 7-Hydroxylase, Cytochrome P450 IIA3, Cytochrome P450 2A6, Cytochrome P450(I), CYPIIA6, CYP2A3, Flavoprotein-Linked Monooxygenase, Xenobiotic Monooxygenase, EC 1.14.14.1, EC 1.14.13., P450C2A, P450PB, CYP2A and CPA6; CYP2B6 also refers non-limitatively to the Cytochrome P450 Family 2 Subfamily B Member 6, Cytochrome P450 Subfamily IIB (Phenobarbital-Inducible) Polypeptide 6, Cytochrome P450 Family 2 Subfamily B Polypeptide 6, 1,4-Cineole 2-Exo-Monooxygenase, Cytochrome P450 IIB1, Cytochrome P450 2B6, CYPIIB6, Cytochrome P450 Subfamily IIB (Phenobarbital-Inducible), Cytochrome P450 Family 2 Subfamily B, EC 1.14.14.1, EC 1.14.13., CYP2B7P, CYP2B7, CYP2B, CPB6, EFVM, IIB1 and P450; CYP2B7P also refers non-limitatively to the Cytochrome P450 Family 2 Subfamily B Member 7 Pseudogene, Cytochrome P450 Family 2 Subfamily B Polypeptide 7 Pseudogene 1, Cytochrome P450 Family 2 Subfamily B Polypeptide 7 Pseudogene, Cytochrome P450 Subfamily IIB (Phenobarbital-Inducible) Polypeptide 7, Cytochrome P450 2B7 Short Isoform, Cytochrome P450 2B7 Isoform, CYP2B7P1, CYP2B7 and CYP2B; CYP2C9 also refers non-limitatively to the Cytochrome P450 Family 2 Subfamily C Member 9, Cytochrome P450 Family 2 Subfamily C Polypeptide 9, Cytochrome P450 PB-1, Cytochrome P450 2C9, Cytochrome P-450MP, CYP2C10, CYPIIC9, Cytochrome P450 Subfamily IIC (Mephenytoin 4-Hydroxylase) Polypeptide 9, Cytochrome P-450 S-Mephenytoin 4-Hydroxylase, Flavoprotein-Linked Monooxygenase, (R)-Limonene 6-Monooxygenase, (S)-Limonene 6-Monooxygenase, (S)-Limonene 7-Monooxygenase, S-Mephenytoin 4-Hydroxylase, Cholesterol 25-Hydroxylase, Microsomal Monooxygenase, Xenobiotic Monooxygenase, Cytochrome P450 MP-4, Cytochrome P450 MP-8, EC 1.14.14.53, EC 1.14.14.51, EC 1.14.14.52, EC 1.14.14.1, EC 1.14.14, P450IIC9, CYP2C and CPC9; CYP2E1 also refers non-limitatively to the Cytochrome P450 Family 2 Subfamily E Member 1, Cytochrome P450 Subfamily IIE (Ethanol-Inducible) Polypeptide 1, Cytochrome P450 Family 2 Subfamily E Polypeptide 1, 4-Nitrophenol 2-Hydroxylase, Cytochrome P450 2E1, Cytochrome P450-J, CYPIIE1, CYP2E, Flavoprotein-Linked Monooxygenase, Microsomal Monooxygenase, Xenobiotic Monooxygenase, EC 1.14.13.n7, EC 1.14.14.1, EC 1.14.14., P450C2E, P450-J and CPE1; CYP3A7 also refers non-limitatively to the Cytochrome P450 Family 3 Subfamily A Member 7, Cytochrome P450 Family 3 Subfamily A Polypeptide 7, Cytochrome P450 Subfamily IIIA Polypeptide 7, Cytochrome P450-HFLA, Cytochrome P450 3A7, CYPIIIA7, P450HLp2, Flavoprotein-Linked Monooxygenase, Aryl Hydrocarbon Hydroxylase, Microsomal Monooxygenase, Xenobiotic Monooxygenase, P-450(HFL33), EC 1.14.14.1, EC 1.14.14., P-4501 11A7, P450-HFLA and CP37; F9 also refers non-limitatively to the Coagulation Factor IX, Plasma Thromboplastin Component, Plasma Thromboplastic Component, Christmas Factor, EC 3.4.21.22, PTC, Factor IX F9, Hemophilia B, Factor IX, EC 3.4.21, Factor 9, F9 P22, THPH8, HEMB, FIX and P19; NAGS also refers non-limitatively to the N-Acetylglutamate Synthase, N-Acetylglutamate Synthase Mitochondrial, Amino-Acid Acetyltransferase, EC 2.3.1.1, AGAS and ARGA; PDX1 also refers non-limitatively to the Pancreatic And Duodenal Homeobox 1, Insulin Promoter Factor 1 Homeodomain Transcription Factor, Somatostatin-Transactivating Factor 1, Pancreas/Duodenum Homeobox Protein 1, Somatostatin Transcription Factor 1, Insulin Upstream Factor 1, Islet/Duodenum Homeobox-1, Glucose-Sensitive Factor, IDX-1, IPF-1, IUF-1, PDX-1, STF-1, IPF1, GSF, Pancreatic-Duodenal Homeobox Factor 1, Insulin Promoter Factor 1, PAGEN1, MODY4, IUF1 and STF1; UGT1A1 also refers non-limitatively to the UDP Glucuronosyltransferase Family 1 Member A1, UDP Glycosyltransferase 1 Family Polypeptide A1, Bilirubin-Specific UDPGT Isozyme 1, UDP-Glucuronosyltransferase 1-1, UDP-Glucuronosyltransferase 1-A, UDP-Glucuronosyltransferase 1A1, EC 2.4.1.17, UDPGT 1-1, HUG-BR1, UGT1-01, UGT-1A, UGT1*1, UGT1.1, UGT1A, GNT1, UGT1, UDP Glucuronosyltransferase 1 Family Polypeptide A1, Bilirubin UDP-Glucuronosyltransferase Isozyme 1, Bilirubin UDP-Glucuronosyltransferase 1-1, Bilirubin UDP-Glucuronosyltransferase, BILIQTL1 and UDPGT.

Illustratively, the UGT1A1 marker is usually associated with ammonia detoxification and bilirubin conjugation. In one embodiment, the hepatic stem cells, in particular within the population of cells, are characterized by the non-expression of the UGT1A1 marker (UGT1A1−), preferably the non-expression of the human UGT1A1 marker. In practice, the said hepatic stem-like cells have an impaired ability to detoxify ammonia and to conjugate bilirubin.

It is understood that ammonia metabolism via the urea cycle is an essential function of hepatocytes in an advanced state of maturation. In some embodiments, ammonia metabolism may be evaluated by absence of expression of urea cycle genes (such as NAGS) or changes in ammonium concentration in the cell culture supernatant over a 24-hour period after addition of ammonium chloride of known concentration. In practice, 1 mM of ammonium chloride standard may be added to the cell culture, supernatant may be collected 24 h upon ammonium chloride addition, and ammonium concentration may be measured, e.g., using a colorimetric ammonia assay kit (BioVision®).

In some embodiments, the hepatic stem-like cells according to the invention express at least one growth factor marker, in particular the hepatocyte growth factor marker (HGF+), and/or at least one cytokine, and/or at least one molecule having anti-inflammatory properties, immunosuppressive properties, anti-fibrotic properties, anti-steatosis properties and/or anti-oxidative stress properties, and the likes. In some embodiments, said hepatic stem-like cell is derived from a precursor cell selected in the group consisting of a pluripotent stem cell (pSC), an induced pluripotent stem cell (ipSC), a multipotent stem cell, a differentiated hepatic cell and a transdifferentiated non-hepatic cell.

As used herein, the term “pluripotent cell” refers to a cell having the capacity to generate a cellular progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism. A standard art-accepted test, such as the ability to form a teratoma in 8 to 12 weeks-old SCID mice, can be used to establish the pluripotency of a cell population. However, identification of various pluripotent stem cell characteristics can also be used to identify pluripotent cells.

In some embodiments of the invention, the pluripotent stem cells are animal pluripotent stem cells, more preferably human pluripotent stem cells.

In certain embodiments, human pluripotent stem cells may express at least two, and optionally all, of the 13 markers selected in the group consisting of SSEA-3, SSEA-4, TRA-I-60, TRA-I-81, TRA-2-49/6E, ALP, SOX2, E-cadherin, UTF-I, OCT4, LIN28, REX1, and NANOG. As used herein, the expression “at least two” includes 2, 3, 4, 5, 6, 7, 8, 9, 10, 11, 12 and 13.

As used herein, an “induced pluripotent stem cell” (iPSC) refers to a pluripotent stem cell artificially derived from a non-pluripotent cell. A non-pluripotent cell may be a cell of lesser ability (or potency) to self-renew and to differentiate as compared to a pluripotent stem cell. Cells of lesser potency may be, but are not limited to, somatic stem cells, tissue specific progenitor cells, primary or secondary cells. iPSCs have been reproducibly obtained by reprogramming different cell types by forced, induced expression and/or overexpression of factors important for embryonic development, proliferation and cell cycle control, in particular the OCT4, SOX2, c-MYC and KLF4 transcription factor cocktail or by an alternative combination of factors, substituting KLF4 and c-MYC by or adding NANOG and LIN28, or any methods known from the skilled man to improve reprogramming process (carrying out the use of small molecules such as DNA methyltransferase (DNMT) inhibitors, miRNAs, vitamin, hypoxia, etc. . . . ).

As used herein, the term “reprogramming” refers to the process of changing the fate of a given cell into that of a different cell type, by the mean of a forced expression of a set of factors (or reprogramming factors) in the given cells. Methods for generating iPSCs based on expression vectors encoding reprogramming factors have been described in the art, e.g., WO2007/69666, EP2096169 and WO2010/042490. In practice, reprogramming may be achieved through the use of expression vectors allowing the ectopic expression of the reprogramming factors, in particular bacterial artificial chromosome (BAC) vectors, cosmid vectors, plasmid vectors, transposon-based vectors (such as PiggyBac), viral vectors, RNA, protein, small molecules and the likes. Suitable expression vectors are disclosed, e.g., in Gonzilez et al. (2011).

In some embodiments, the iPSCs are animal iPSCs, more preferably human iPSCs (hiPSCs). In certain embodiments, the iPSCs, preferably the hiPSCs, are derived from cells obtained indifferently from a healthy subject or from a subject with a liver disorder. In some embodiments, the iPSCs, preferably the hiPSCs, are derived from cells obtained from an individual with no liver disorder, in particular with no chronic liver disorder. In some alternative embodiments, the iPSCs, preferably the hiPSCs, are derived from cells obtained from an individual with a liver disorder, in particular a chronic liver disorder, with the proviso that the iPSCs, preferably the hiPSCs, are not deriving from hepatic cells. In practice, the choice of iPSCs, in particular hiPSCs, may be advantageous for performing autologous transplantation. Non-limitative examples of sources of iPSCs are peripheral blood mononuclear cells (PBMCs), fibroblasts, mesenchymal stem cells, urinary cells and the likes.

In practice, iPSCs may be commercially available, e.g., from ATCC®. Non-limitative examples of iPSCs are: iPSCs derived from foreskin fibroblasts (ATCC® ACS-7030); sendai virus reprogrammed hiPSC from bone marrow CD34+ cells (ATCC® ACS-1027; ATCC® ACS-1028; ATCC® ACS-1029; ATCC® ACS-1030; ATCC® ACS-1031); Yamanaka retrovirus reprogrammed hiPSC from dermal fibroblast (ATCC® ACS-1023); iPSC-derived Mesenchymal Stem Cells (ATCC® ACS-7010); sendai virus reprogrammed hiPSC from hepatic fibroblast (ATCC® ACS-1020); sendai virus reprogrammed hiPSC from cardiac fibroblast (ATCC® ACS-1021).

In some embodiments, a population of cells comprising hepatic stem-like cells according to the invention may be obtained from the differentiation of multipotent cells, such as mesenchymal stem cells, optionally on a solid support.

As used herein, the term “multipotent” refers to cells capable of differentiating into at least two terminally differentiated cell types. In some embodiments, the multipotent cells according to the invention are animal multipotent cells, more preferably human multipotent cells.

As used herein, “mesenchymal stem cells” (MSCs) generally refer to stromal cells from a specialized tissue (also named differentiated tissue) and capable of self-renewal (i.e. making identical copies of themselves) for the lifetime of the organism and have multipotent differentiation potential. In some embodiments, the MSCs according to the invention are animal MSCs, more preferably human MSCs (hMSCs).

In practice, hMSCs suitable for implementing the instant invention thus encompass any suitable human multipotent stem cells derived from any suitable tissue, using any appropriate isolation method.

Illustratively, hMSCs encompass, but are not limited to, adult multilineage inducible (MIAMI) cells (D'Ippolito et al.; 2004), cord blood derived stem cells (Kogler et al.; 2004), mesoangioblasts (Sampaolesi et al.; 2006; Dellavalle et al.; 2007), and amniotic stem cells (De Coppi et al.; 2007). Furthermore, umbilical cord blood banks (e.g., Etablissement Français du Sang, France) provide secure and easily available sources of such cells for transplantation. hMSCs may be commercially available, e.g., from CREATIVE BIOARRAY®. Non-limitative examples of hMSCs are: HMSC.BM-100; HMSC.AD-100; Human Mesenchymal Stem Cells-Adult(HMSC-Ad); Human Mesenchymal Stem Cells Wharton's Jelly (HMSC-WJ); Human Mesenchymal Stem Cells from Umbilical Cord Matrix (hMSC-UC); Human Mesenchymal Stem Cells-adipose (HMSC-ad); Human Mesenchymal Stem Cells-bone marrow (HMSC-bm); Human Mesenchymal Stem Cells-hepatic (HMSC-he).

In certain embodiments, a population of cells comprising hepatic stem-like cells according to the invention may be obtained from differentiated hepatic cells, i.e., the differentiation of cells isolated from adult livers (e.g., hepatocyte progenitor cells). In some embodiments, the differentiated hepatic cells are animal differentiated hepatic cells, more preferably human differentiated hepatic cells.

In certain embodiments, a population of cells comprising hepatic stem-like cells according to the invention may be obtained from transdifferentiated non-hepatic cells, i.e., the conversion of somatic cells such as fibroblasts. In some embodiments, the transdifferentiated hepatic cells are animal transdifferentiated hepatic cells, more preferably human transdifferentiated hepatic cells.

In some embodiments, said pluripotent stem cells (pSCs) are obtained from embryonic stem cells (ESCs), preferably from human embryonic stem cells (hESCs).

As used herein, “embryonic stem cells” refer to embryonic cells, which are capable of differentiating into cells of any one of the three embryonic germ layers, namely endoderm, ectoderm or mesoderm, or maintaining in an undifferentiated state. Such cells may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763), embryonic germ (EG) cells which are obtained from the genital tissue of a fetus any time during gestation, preferably before 10 weeks of gestation and other methods with non-fertilized eggs, such as parthenogenesis method or nuclear transfer.

In practice, suitable embryonic stem cells may be obtained using well-known cell-culture methods. For example, hESCs can be isolated from human blastocysts. Human blastocysts are typically obtained from human in vivo preimplantation embryos or from in vitro fertilized (IVF) embryos. Alternatively, a single cell human embryo can be expanded to the blastocyst stage. Further details on methods of preparation hESCs may be found in U.S. Pat. No. 5,843,780.

In practice, hESCs may advantageously be obtained without embryo destruction, as described by Chung et al. (2008), or by parthenogenetic activation of an unfertilized oocyte, as described by Sagi et al. (2016).

In some aspects, the invention further relates to cells derived from hepatic stem-like cells according to the instant invention. In some embodiments, cells derived from hepatic stem-like cells according to the instant invention include hepatocyte-like cells (HLCs) and cholangiocytes. As used herein, the term “cholangiocyte” is intended to refer to epithelial cells of the bile duct. As used herein, the cells derived from hepatic stem-like cells according to the instant invention constitute the progeny of said hepatic stem-like cells.

As used herein, the term “extract thereof” refers to an extract of hepatic stem-like cells, or the population, in particular isolated population, of cells comprising hepatic stem-like cells according to the invention. The term “extract” refers to any cellular fraction or culture supernatant obtained from a culture of hepatic stem-like cells, or a population, in particular isolated population, of cells comprising hepatic stem-like cells according to the invention, provided that the extract would conserve the properties of the hepatic stem-like cells, in particular their therapeutic properties.

Cellular fractions may be obtained according to any suitable method known from the state in the art, or a method adapted therefrom. Obtaining cellular fractions may include mechanical, chemical and/or enzymatic cellular lysis, centrifugation, ultracentrifugation, affinity chromatography. Cellular fractions encompass cytosolic fraction, cytoplasmic fraction, membrane fractions, soluble fractions, insoluble fractions, vesicles, exosomes, and combination thereof.

In some embodiments, the extract of the hepatic stem-like cells, or the population, in particular isolated population, of cells comprising or consisting of hepatic stem-like cells according to the invention comprises exosomes or exosome-like vesicles.

As used herein, the term “Exosome” may refer to endocytic-derived nanovesicles that are naturally secreted by nearly all cell types in the body. The exosomes are lipidic vesicles that comprise proteins, nucleic acids, and lipids. In practice, the exosomes may be collected, isolated and/or purified according to any suitable method known in the state of the art, or a method adapted therefrom.

Illustratively, the exosomal fraction may be isolated by differential centrifugation from culture medium; by polymer precipitation; by high-performance liquid chromatography (HPLC). Non-limitative example of differential centrifugation method from culture medium may include the following steps:
1) centrifugation for 10-20 min at a speed of about 300×g to about 500×g, so as to remove cells;
2) centrifugation for 10-20 min at a speed of about 1,500×g to about 3,000×g, so as to remove dead cells;
3) centrifugation for 20-45 min at a speed of about 7,500×g to about 15,000×g, so as to remove cell debris;
4) one or more ultracentrifugation for 30-120 min at a speed of about 100,000×g to about 200,000×g, so as to pellet the exosomes.

Alternatively, isolation of exosomes or exosome-like vesicles may be performed by the mean of a commercial kit, such as, e.g., the exoEasy Maxi Kit (QIAGEN®) or the Total Exosome Isolation Kit (THERMOFISHER SCIENTIFIC®).

In some embodiments, the exosomes or the exosome-like vesicles have an average diameter ranging from about 1 nm to about 250 nm, preferably from about 20 nm to about 200 nm, more preferably from about 90 nm to 150 nm. Within the scope of the instant invention, the expression “from about 1 nm to about 250 nm” includes 1, 2, 3, 4, 5, 6, 7, 8, 9, 10, 15, 25, 30, 35, 40, 45, 50, 55, 60, 65, 70, 75, 80, 85, 90, 95, 100, 105, 110, 115, 120, 125, 130, 135, 140, 145, 150, 160, 170, 180, 190, 200, 210, 220, 230, 240 and 250 nm.

In some embodiments, the extract obtained from the hepatic stem-like cells according to the invention further comprises the alpha-fœtoprotein marker (AFP+) and not expressing the albumin marker (ALB−).

In some embodiments, the extract obtained from the hepatic stem-like cells according to the invention further comprises the T-Box Transcription Factor 3 marker (TBX3+) and/or the Hepatocyte Nuclear Factor 4 Alpha marker (HNF4A+), preferably the T-Box Transcription Factor 3 marker (TBX3+) and the Hepatocyte Nuclear Factor 4 Alpha marker (HNF4A+).

In certain embodiments, the extract obtained from the hepatic stem-like cells according to the invention further comprises at least one marker, in particular at least two markers, more particularly three markers, preferably human markers, selected in the group consisting of KRT19, EPCAM and TTR. In some embodiments, the extract obtained from the hepatic stem-like cells according to the invention further comprises at least one marker, in particular at least two markers, more particularly three markers, even more particularly four markers, preferably human markers, selected in the group consisting of KRT19, EPCAM, TTR and HGF. In some embodiments, the extract obtained from the hepatic stem-like cells according to the invention further comprises at least one marker, in particular at least two markers, more particularly three markers, preferably human markers, selected in the group consisting of KRT19, EPCAM and TTR; and/or further express HGF, preferably human HGF.

In some embodiments, the extract obtained from the hepatic stem-like cells according to the invention further comprises the AFP, TBX3 and HNF4A markers, and does not comprise the ALB marker. In certain embodiments, the extract obtained from the hepatic stem-like cells according to the invention further comprises the AFP, TBX3, HNF4A and HGF markers, and does not comprise the ALB marker. In some embodiments, the extract obtained from the hepatic stem-like cells according to the invention further comprises the AFP, TBX3, HNF4A and TTR markers, and does not comprise the ALB marker. In certain embodiments, the extract obtained from the hepatic stem-like cells according to the invention further comprises the AFP, TBX3, HNF4A, HGF and TTR markers, and does not comprise the ALB marker. In some embodiments, the extract obtained from the hepatic stem-like cells according to the invention further comprises the AFP, TBX3, HNF4A and EPCAM markers, and does not comprise the ALB marker. In certain embodiments, the extract obtained from the hepatic stem-like cells according to the invention further comprises the AFP, TBX3, HNF4A, EPCAM and HGF markers, and does not comprise the ALB marker. In some embodiments, the extract obtained from the hepatic stem-like cells according to the invention further comprises the AFP, TBX3, HNF4A, EPCAM, TTR and KRT19 markers, and does not comprise the ALB marker. In certain embodiments, the extract obtained from the hepatic stem-like cells according to the invention further comprises the AFP, TBX3, HNF4A, EPCAM, TTR, KRT19, and HGF markers, and does not comprise the ALB marker. Preferably the marker is a human marker.

In some embodiments, the extract obtained from the hepatic stem-like cells according to the invention may comprise one or more of markers selected in the group consisting of ACTB, ATG1, AFP, ANXA2, ANXA5, ANXA6, APOA1, APOA2, APOA4, APOB, APOC3, APOE, BMP2, BMP4, CD164, CD24, CD63, CD81, CD9, CD99, CLTC, CXCR4, DCN, DLKT, DPP4, EEF1A1, EEF2, ENO1, EPCAM, FGF19, FOXA2, GAPDH, GATA4, GATA6, GJA1, GPC3, GSTA1, GSTA2, HGF, HMOX1, HNF1B, HNF4A, HSP90AA1, HSP90AB, HSPA8, HSPG2, IGF1, IGFBP3, IL6ST, ITGA6, KRT18, KRT19, KRT8, LCP1, MKI67, MYDGF, NODAL, PKM, PITX2, PROX1, SEPP1, SERPINA1, SMAD7, SNAI2, SOD1, SOX17, SPARC, TBX3, TFRC, TUBA1A, TUBB, TUBB3, TUBB6, TUBB4A, TUBB4B, TUBA1B, TUBB2A, TUBB2B, TTR, UGT3A1, VIM, and VTN; and/or may not comprise one or more of markers selected in the group consisting of ALB, ABCB11, ASGR1, CYP1A2, CYP2A6, CYP2B6, CYP2B7P, CYP2C9, CYP2E1, CYP3A4, CYP3A7, F9, NAGS, PDX1, UGT1A1.

The invention further relates to a method for generating hepatic stem-like cells, as disclosed herein, comprising the steps of:
a) culturing definitive endoderm cells in an induction culture medium, so as to generate hepatic stem-like cells expressing the AFP marker (AFP+) and not expressing the ALB marker (ALB−);
b) isolating the hepatic stem-like cells generated at step a).

In some embodiments, the isolated hepatic stem-like cells constitute a population of cells.

As used herein, “definitive endoderm cells” refer to cells expressing phenotypic markers that are characteristic of the definitive endoderm differentiation phase, including but not limited to the SOX17 and the FOXA2 markers. In addition, definitive endoderm cells are not expressing the ALB marker (ALB−).

As used herein, an “induction culture medium” refers to a culture medium that is capable of inducing differentiation of definitive endoderm cells into hepatic stem-like cells, as defined by the instant invention.

In practice, a “culture medium” refers to the generally accepted definition in the field of cellular biology, i.e., any medium suitable for promoting the growth of the cells of interest. In some embodiments, a suitable culture medium may include a chemically defined medium, i.e., a nutritive medium only containing specified components, preferably components of known chemical structure.

In some embodiments, a chemically defined medium may be a serum-free and/or feeder-free medium. As used herein, a “serum-free” medium refers to a culture medium containing no added serum. As used herein, a “feeder-free” medium refers to a culture medium containing no added feeder cells.

A suitable culture medium for use according to the invention may be an aqueous medium that may include a combination of substances such as one or more salts, carbon sources, amino acids, vitamins, minerals, reducing agents, buffering agents, lipids, nucleosides, antibiotics, cytokines, and growth factors. Examples of suitable culture media include, without being limited to RPMI medium, William's E medium, Basal Medium Eagle (BME), Eagle's Minimum Essential Medium (EMEM), Minimum Essential Medium (MEM), Dulbecco's Modified Eagles Medium (DMEM), Ham's F-10, Ham's F-12 medium, Kaighn's modified Ham's F-12 medium, DMEM/F-12 medium, and McCoy's 5A medium, which may be further supplemented with any one of the above-mentioned substances. In some embodiments, a culture medium according to the invention may be a synthetic culture medium such as the RPMI (Roswell Park Memorial Institute medium) or the CMRL-1066 (Connaught Medical Research Laboratory).

In practice, the media may be supplemented with additional additives. Illustratively, the commercial B-27 supplement from INVITROGEN® may represent a suitable supplement, as it comprises insulin, albumin, superoxide dismutase (SOD), catalase and other anti-oxidants (GSH), and unique fatty acids, such as linoleic acid, linolenic acid and lipoic acid.

In some embodiments, step a) is performed for about 5 days to 8 days in an induction culture medium comprising a bone morphogenetic protein, preferably BMP4 and/or comprising a fibroblast growth factor, preferably FGF10, and optionally comprising the hepatocyte growth factor HGF and/or a GSK3 inhibitor, preferably CHIR-99021.

In some embodiments, the bone morphogenetic protein (BMP) is selected in a group of growth factors that are members of the TGF-beta superfamily comprising molecules activating AR Smads, such as, e.g., Activin A, Activin B, Activin C, Activin E, GDF-8/Myostatin, Nodal, TGF-beta 1, TGF-beta 2, TGF-beta 3; and molecules activating BR Smads, such as, e.g., BMP2, BMP4, BMP6, BMP8a, BMP8b, GDF5, GDF6, GDF7, AMH. Suitable BMPs according to the invention are, e.g., disclosed in Miyazono et al. (2019).

In some embodiments, the fibroblast growth factor (FGF) is selected in a group comprising a FGF from the FGF1 subfamily, including FGF1 (also named aFGF), FGF2 (also named bFGF); a FGF from the FGF4 subfamily, including FG4, FGF5, FGF6; a FGF from the FGF7 subfamily, including FGF3, FGF7, FGF10, FGF22; a FGF from the FGF8 subfamily, including FGF8, FGF17, FGF18; a FGF from the FGF9 subfamily, including FGF9, FGF16, FGF20; a FGF from the FGF11 subfamily, including FGF11, FGF12, FGF13, FGF14; and a FGF from the FGF19 subfamily, including FGF15/19, FGF21, FGF23. In some embodiments, the fibroblast growth factor (FGF) is selected in the group consisting of FGF1, FGF2, FGF3, FGF4, FGF5, FGF6, FGF7, FGF8, FGF9, FGF10, FGF11, FGF12, FGF13, FGF14, FGF16, FGF17, FGF18, FGF15/19, FGF20, FGF21, FGF22 and FGF23.

In practice, the bone morphogenetic protein (BMP) and/or the fibroblast growth factor (FGF) is/are present in the induction medium at a concentration from about 0.01 ng/ml to about 500 ng/ml, preferably from about 0.5 ng/ml to about 250 ng/ml, more preferably from about 1 ng/ml to about 50 ng/ml. Within the scope of the instant invention, the expression “from about 0.01 ng/ml to about 500 ng/ml” encompasses 0.01 ng/ml, 0.05 ng/ml, 0.1 ng/ml, 0.5 ng/ml, 1.0 ng/ml, 1.5 ng/ml, 2.0 ng/ml, 2.5 ng/ml, 5.0 ng/ml, 7.5 ng/ml, 10.0 ng/ml, 12.5 ng/ml, 15.0 ng/ml, 17.5 ng/ml, 20 ng/ml, 25 ng/ml, 30 ng/ml, 35 ng/ml, 40 ng/ml, 45 ng/ml, 50 ng/ml, 55 ng/ml, 60 ng/ml, 70 ng/ml, 80 ng/ml, 90 ng/ml, 100 ng/ml, 150 ng/ml, 200 ng/ml, 250 ng/ml, 300 ng/ml, 350 ng/ml, 400 ng/ml, 450 ng/ml and 500 ng/ml.

In some embodiments, BMP4 is comprised in the induction medium in a concentration of from about 0.1 ng/ml to about 100 ng/ml, preferably from about 0.5 ng/ml to about 50 ng/ml, more preferably from about 1 ng/ml to about 25 ng/ml.

In some embodiments, FGF10 is comprised in the induction medium in a concentration of from about 0.1 ng/ml to about 100 ng/ml, preferably from about 0.5 ng/ml to about 50 ng/ml, more preferably from about 1 ng/ml to about 25 ng/ml.

Within the scope of the instant invention, the expression “from about 0.1 ng/ml to about 100 ng/ml” encompasses 0.1 ng/ml, 0.5 ng/ml, 1.0 ng/ml, 1.5 ng/ml, 2.0 ng/ml, 2.5 ng/ml, 5.0 ng/ml, 7.5 ng/ml, 10.0 ng/ml, 12.5 ng/ml, 15.0 ng/ml, 17.5 ng/ml, 20 ng/ml, 25 ng/ml, 30 ng/ml, 35 ng/ml, 40 ng/ml, 45 ng/ml, 50 ng/ml, 55 ng/ml, 60 ng/ml, 70 ng/ml, 80 ng/ml, 90 ng/ml and 100 ng/ml.

When present in the induction medium, the hepatocyte growth factor HGF is comprised in a concentration of from about 0.5 ng/ml to about 150 ng/ml, preferably from about 1 ng/ml to about 100 ng/ml, more preferably from about 5 ng/ml to about 50 ng/ml. Within the scope of the instant invention, the expression “from about 0.5 ng/ml to about 150 ng/ml” encompasses 0.5 ng/ml, 1.0 ng/ml, 1.5 ng/ml, 2.0 ng/ml, 2.5 ng/ml, 5.0 ng/ml, 7.5 ng/ml, 10.0 ng/ml, 12.5 ng/ml, 15.0 ng/ml, 17.5 ng/ml, 20 ng/ml, 25 ng/ml, 30 ng/ml, 35 ng/ml, 40 ng/ml, 45 ng/ml, 50 ng/ml, 55 ng/ml, 60 ng/ml, 70 ng/ml, 80 ng/ml, 90 ng/ml, 100 ng/ml, 110 ng/ml, 120 ng/ml, 130 ng/ml, 140 ng/ml and 150 ng/ml.

Within the scope of the invention, for about 5 days to about 8 days encompasses 5, 6, 7 and 8 days.

In some embodiments, step a) may be preceded by step al) comprising culturing pluripotent stem cells (PSCs), induced pluripotent stem cells (iPSCs) or multipotent stem cells, in a culture medium so as to generate definitive endoderm cells expressing the FOXA2 and the SOX17 markers. Noticeably, definitive endoderm cells are not expressing the ALB marker (ALB−).

In certain embodiments, step al) may be performed for about 3 days to about 6 days in a culture medium comprising a GSK3 inhibitor, preferably CHIR-99021 and optionally comprising a Transforming Growth Factor-beta compound, preferably ACT-A, and/or an activator of the Wnt signaling pathway, preferably Wnt3A.

Within the scope of the invention, for about 3 days to about 6 days encompasses 3, 4, 5 and 6 days.

In some embodiments, the GSK3 inhibitor is selected in a group comprising 3F8 (CAS No. 159109-11-2), Alsterpaullone (CAS No. 237430-03-4), CHIR-98014 (CAS No. 252935-94-7), CHIR-99021 (CAS No. 1797989-42-4), Indirubin-3′-oxime (CAS No. 160807-49-8), Kenpaullone (CAS No. 142273-20-9), SB216763 (CAS No. 280744-09-4), TC-G 24 (CAS No. 1257256-44-2) TCS 2002 (CAS No. 1005201-24-0) and TWS119 (CAS No. 601514-19-6), lithium, copper, mercury, tungsten, zinc curcumin, beryllium, 6-BIO, dibromocantharelline, hymenialdesine, indirubin, meridianin, CT98014, CT98023, CT99021, SB-41528, AR-A014418, AZD-1080, Cazpaullone, Manzamine A, Palinurine, Tricantine, TDZD-8, NP00111, NP031115, Tideglusib, HMK-32, L803-mts, valproic acid, curcumin, aloisines, IM-12, LY2090314. In practice, GSK3 inhibitors may be commercially available, e.g., from SANTA CRUZ BIOTECHNOLOGY®, SELLECKCHEM® and TOCRIS®.

In some embodiments, the GSK3 inhibitor is present in the culture medium in a concentration of from about 0.01 μM to about 50 μM. Within the scope of the instant invention, the expression “from about 0.01 μM to about 50 μM” encompasses 0.01 μM, 0.02 μM, 0.03 μM, 0.04 μM, 0.05 μM, 0.06 μM, 0.07 μm, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 1.5 μM, 2.0 μM, 2.5 μM, 3.0 μM, 3.5 μM, 4.0 μM, 4.5 μM, 5.0 μM, 5.5 μM, 6.0 μM, 6.5 μM, 7.0 μM, 7.5 μM, 8 μM, 9 μM, 10 μM, 11 μM, 12 μM, 13 μM, 14 μM, 15 μM, 20 μM, 25 μM, 30 μM, 35 μM, 40 μM, 45 μM and 50 μM.

In some embodiments, the GSK3 inhibitor is CHIR-99021. In some embodiments, CHIR-99021 is comprised in the culture medium in a concentration of from about 0.1 μM to about 15 μM, preferably from about 0.5 μM to about 10 μM, more preferably from about 1 μM to about 5 μM. Within the scope of the instant invention, the expression “from about 0.1 μM to about 15 μM” encompasses 0.1 μM, 0.2 μM, 0.3 μM, 0.4 μM, 0.5 μM, 0.6 μM, 0.7 μM, 0.8 μM, 0.9 μM, 1.0 μM, 1.5 μM, 2.0 μM, 2.5 μM, 3.0 μM, 3.5 μM, 4.0 μM, 4.5 μM, 5.0 μM, 5.5 μM, 6.0 μM, 6.5 μM, 7.0 μM, 7.5 μM, 8.0 μM, 9.0 μM, 10.0 μM, 11.0 μM, 12.0 μM, 13.0 μM, 14.0 μM and 15 μM.

In some embodiments, the Transforming Growth Factor-beta compound is selected in a group comprising Activin A, Activin B, Activin C, Activin E, AMH, BMP2, BMP4, BMP6, BMP8a, BMP8b, GDF5, GDF6, GDF7, GDF-8/Myostatin, Nodal, TGF-beta 1, TGF-beta 2, TGF-beta 3.

In practice, the Transforming Growth Factor-beta is present in the culture medium at a concentration from about 0.01 ng/ml to about 1,000 ng/ml, preferably from about 0.5 ng/ml to about 500 ng/ml, more preferably from about 1 ng/ml to about 250 ng/ml. Within the scope of the instant invention, the expression “from about 0.01 ng/ml to about 1,000 ng/ml” encompasses 0.01 ng/ml, 0.05 ng/ml, 0.1 ng/ml, 0.5 ng/ml, 1.0 ng/ml, 1.5 ng/ml, 2.0 ng/ml, 2.5 ng/ml, 5.0 ng/ml, 7.5 ng/ml, 10.0 ng/ml, 12.5 ng/ml, 15.0 ng/ml, 17.5 ng/ml, 20 ng/ml, 25 ng/ml, 30 ng/ml, 35 ng/ml, 40 ng/ml, 45 ng/ml, 50 ng/ml, 55 ng/ml, 60 ng/ml, 70 ng/ml, 80 ng/ml, 90 ng/ml, 100 ng/ml, 150 ng/ml, 200 ng/ml, 250 ng/ml, 300 ng/ml, 350 ng/ml, 400 ng/ml, 450 ng/ml, 500 ng/ml, 600 ng/ml, 700 ng/ml, 800 ng/ml, 900 ng/ml and 1,000 ng/ml.

In some embodiments, the Transforming Growth Factor-beta compound is activin A (ACT-A). When present in the culture medium, ACT-A is comprised in a concentration of from about 1 ng/ml to about 500 ng/ml, preferably from about 25 ng/ml to about 250 ng/ml, more preferably from about 50 ng/ml to about 150 ng/ml. Within the scope of the instant invention, the expression “from about 1 ng/ml to about 500 ng/ml” encompasses 1 ng/ml, 5 ng/ml, 10 ng/ml, 20 ng/ml, 25 ng/ml, 50 ng/ml, 75 ng/ml, 100 ng/ml, 150 ng/ml, 200 ng/ml, 250 ng/ml, 300 ng/ml, 350 ng/ml, 400 ng/ml, 450 ng/ml and 500 ng/ml.

In some embodiments, the activator of the Wnt signaling pathway is selected in the group of the Wnt family consisting of Wnt-1 (also referred to as Int-1), Wnt-2 (also referred to as Irp (Int-1-related Protein)), Wnt-2b (also referred to as Wnt-13), Wnt-3 (referred to as Int-4), Wnt-3a, Wnt-4, Wnt-5a, Wnt-5b, Wnt-6, Wnt-7a, Wnt-7b, Wnt-8a (referred to as Wnt-8d), Wnt-8b, Wnt-9a (referred to as Wnt-14), Wnt-9b (referred to as Wnt-14b or Wnt-15), Wnt-10a, Wnt-10b (referred to as Wnt-12), Wnt-11, Wnt-12 (also referred to as Wnt-10b), Wnt-13 (also referred to as Wnt-2b), Wnt-14 (also referred to as Wnt-9a), Wnt-14b (also referred to as Wnt-9ab), Wnt-15 (also referred to as Wnt-9b) and Wnt-16.

In practice, the activator of the Wnt signaling pathway is present in the culture medium at a concentration from about 0.01 ng/ml to about 1,000 ng/ml, preferably from about 0.5 ng/ml to about 500 ng/ml, more preferably from about 1 ng/ml to about 250 ng/ml.

In some embodiments, the activator of the Wnt signaling pathway is Wnt-3a. When present in the culture medium, Wnt-3a is comprised in a concentration of from about 1 ng/ml to about 250 ng/ml, preferably from about 5 ng/ml to about 150 ng/ml, more preferably from about 25 ng/ml to about 100 ng/ml. Within the scope of the instant invention, the expression “from about 1 ng/ml to about 250 ng/ml” encompasses 1 ng/ml, 5 ng/ml, 10 ng/ml, 15 ng/ml, 20 ng/ml, 25 ng/ml, 50 ng/ml, 75 ng/ml, 100 ng/ml, 125 ng/ml, 150 ng/ml, 175 ng/ml, 200 ng/ml, 225 ng/ml and 250 ng/ml.

In practice, step b) may be performed by any suitable method known in the art, e.g., by FACS, and optionally, one or more wash(es) of the cells in an appropriate medium (culture medium or suitable cellular buffer) may be performed to remove unwanted ingredients from the culture medium.

In certain embodiments, step a) may be followed by, and step b) may be preceded by, step b1) comprising the stripping of the cells from the culture vessel used to perform step a). In practice, the stripping may be performed by chemical and/or enzymatic stripping, including contacting the cells with EDTA and/or trypsin; and/or by mechanical stripping, including scrapping with a suitable tool (e.g., a spatula), or by creating an ebb and flow.

The inventors observed that the hepatic stem-like cells according to the invention are easily handled, when compared to hepatocyte-like cells (HLCs), which present the mature characteristics of hepatic cells within a functional healthy liver. In fact, HLCs generated in vitro strongly adhere to each other and to the culture vessel. Collecting the HLCs therefore requires harsh conditions of chemical and/or enzymatic and/or mechanical stripping. At the industrial scale, the mechanical stripping is often not possible to implement, which results in a negative impact on the yield. Contrarily to HLCs, the hepatic stem-like cells according to the invention are more easily recovered from the culture vessel used to generate them, as loose to moderate stripping conditions, e.g., by chemical stripping, are sufficient to recover more than 90% of the hepatic stem-like cells.

As used herein, loose or moderate chemical and/or enzymatic stripping conditions include the use of trypsin at a final concentration up to at most about 0.5% (v/v) and/or up to at most about 1 mM EDTA.

In certain embodiments, the enzymatic stripping of the hepatic stem-like cells according to the invention from the culture vessel comprises contacting said cells with from about 0.0125% to about 0.5% trypsin. Within the scope of the instant invention, the term “from about 0.0125% to about 0.5% trypsin” includes 0.0125%, 0.015%, 0.0175%, 0.02%, 0.0225%, 0.025%, 0.0275%, 0.03%, 0.0325%, 0.035%, 0.0375%, 0.04%, 0.0425%, 0.045%, 0.0475%, 0.05%, 0.0525%, 0.055%, 0.0575%, 0.06%, 0.0625%, 0.0650%, 0.0675%, 0.07%, 0.0725%, 0.075%, 0.0775%, 0.08%, 0.0825%, 0.085%, 0.0875%, 0.09%, 0.0925%, 0.095%, 0.0975%, 0.1%, 0.125%, 0.15%, 0.175%, 0.2%, 0.225%, 0.25%, 0.275%, 0.3%, 0.325%, 0.35%, 0.375%, 0.4%, 0.425%, 0.45%, 0.475% and 0.5%.

In some embodiments, trypsin may be commercially available, e.g., from THERMOFISCHER SCIENTIFIC®, such as TrypLE™ Express or TrypLE™ Select.

In certain embodiments, the chemical stripping of the hepatic stem-like cells according to the invention from the culture vessel comprises contacting said cells with from about 0.01 mM to about 1 mM EDTA. Within the scope of the instant invention, the term “from about 0.01 mM to about 1 mM EDTA” include 0.01 mM, 0.02 mM, 0.03 mM, 0.04 mM, 0.05 mM, 0.06 mM, 0.07 mM, 0.08 mM, 0.09 mM, 0.1 mM, 0.15 mM, 0.2 mM, 0.25 mM, 0.3 mM, 0.35 mM, 0.4 mM, 0.45 mM, 0.5 mM, 0.55 mM, 0.6 mM, 0.65 mM, 0.7 mM, 0.75 mM, 0.8 mM, 0.85 mM, 0.9 mM, 0.95 mM and 1 mM EDTA.

In some embodiments, the pluripotent stem cells (PSCs) are induced pluripotent stem cells (iPSCs) or embryonic stem cells (ESCs), preferably embryonic stem cells (ESCs), more preferably human embryonic stem cells (hESCs).

In practice, the culture parameters such as the temperature, the pH, the salinity, and the levels of O₂ and CO₂ are adjusted accordingly to the standards established in the state of the art.

In some embodiments, the level of CO₂ during the course of culture is maintained constant and ranges from about 1% to about 10%, preferably from about 2.5% to about 7.5%. Within the scope of the instant invention, the expression “from about 1% to about 10%” encompasses 1%, 1.5%, 2%, 2.5%, 3%, 3.5%, 4%, 4.5%, 5%, 5.5%, 6%, 6.5%, 7%, 7.5%, 8%, 8.5%, 9%, 9.5% and 10%.

Illustratively, the temperature for culturing the cells according to the invention may range from about 30° C. to about 42° C., preferably from about 35° C. to about 40° C., and more preferably from about 36° C. to about 38° C. Within the scope of the invention, the expression “from about 30° C. to about 42° C.” encompasses 30° C., 31° C., 32° C., 33° C., 34° C., 35° C., 36° C., 37° C., 38° C., 39° C., 40° C., 41° C. and 42° C.

In certain embodiments, the culture medium is changed at least every other day, preferably every day, during the course of the culture. In practice, the culture medium is removed, the cells may be washed once or twice with fresh culture medium and a fresh culture medium is provided to the cells.

In some embodiments, the culture of cells in a suitable culture medium, so as to generate hepatic stem-like cells, may be performed in the presence of a matrix, e.g., an extracellular matrix.

As used herein, the term “matrix” refers to a component/material, natural, synthetic or a combination thereof, forming a polymeric network, which provides to in vitro cultured cells (e.g., on culture vessel such as flat plasticware) an in vivo like morphology and physiologically relevant environments. In other words, the matrix, in particular the extracellular matrix, provides the cells to be cultured with a more realistic environment, intended to strengthen the intercellular interactions, to facilitate cell attachment, and to improve cellular growth and differentiation.

In some embodiments, the matrix, in particular the extracellular matrix may comprise at least one ingredient selected in the group consisting of a laminin, a collagen, a fibronectin, a gelatin and a mixture thereof.

In certain embodiments, the matrix, in particular the extracellular matrix, comprises or consists of at least one laminin, preferably wherein said at least one laminin is selected from the group consisting of laminin-111 (LN-111), laminin-211 (LN-211), laminin-332 (LN-332), laminin-411 (LN-411), laminin-421 (LN-421), laminin-511 (LN-511) and laminin-521 (LN-521) and functional fragments thereof. As used herein, the term “functional fragments” refers to fragments of laminin that reproduce the biological function of the full-length laminin protein.

In some embodiments, said laminin is an animal laminin, preferably a human laminin, more preferably a human recombinant laminin. As used herein, the term “recombinant” refers to a laminin which is produced by expression from a corresponding encoding nucleic acid. Systems for cloning and expression of a polypeptide in a variety of different host cells are well known in the art. In practice, recombinant human laminins, such as e.g., recombinant human LN-111 or LN-521, may be commercially available from BIOLAMINA®.

In some embodiments of the invention, the laminin may be coated to a solid support (culture vessel), such as a plate (e.g., a Petri dish) or a vial, in a concentration ranging from about 0.02 μg/ml to about 50 μg/ml, preferably from about 0.1 μg/ml to about 10 μg/ml, more preferably about 5 μg/ml. Within the scope of the instant invention, the expression “from about 0.02 μg/ml to about 50 μg/ml” encompasses 0.02 μg/ml, 0.05 μg/ml, 0.1 μg/ml, 0.5 μg/ml, 1.0 μg/ml, 1.5 μg/ml, 2.0 μg/ml, 2.5 μg/ml, 5.0 μg/ml, 7.5 μg/ml, 10.0 μg/ml, 12.5 μg/ml, 15.0 μg/ml, 17.5 μg/ml, 20 μg/ml, 25 μg/ml, 30 μg/ml, 35 μg/ml, 40 μg/ml, 45 μg/ml and 50 μg/ml.

In some embodiments of the invention, the functional fragment of laminin may be coated to a solid support (culture vessel), such as a plate (e.g., a Petri dish) or a vial, in a concentration ranging from about 0.02 μg/ml to about 100 μg/ml, preferably from about 0.1 μg/ml to about 50 μg/ml, more preferably about 25 μg/ml. Within the scope of the instant invention, the expression “from about 0.02 μg/ml to about 100 μg/ml” encompasses 0.02 μg/ml, 0.05 μg/ml, 0.1 μg/ml, 0.5 μg/ml, 1.0 μg/ml, 1.5 μg/ml, 2.0 μg/ml, 2.5 μg/ml, 5.0 μg/ml, 7.5 μg/ml, 10.0 μg/ml, 12.5 μg/ml, 15.0 μg/ml, 17.5 μg/ml, 20 μg/ml, 25 μg/ml, 30 μg/ml, 35 μg/ml, 40 μg/ml, 45 μg/ml, 50 μg/ml, 60 μg/ml, 70 μg/ml, 80 μ/ml, 90 μg/ml and 100 μg/ml.

In certain embodiments, the matrix, in particular the extracellular matrix may comprise or consist of a mixture of LN-521 and LN-111, in particular, in a respective ratio of about 5%/95%; 10%/90%; 20%/80%; 25%/75%; 30%/70%; 40%/60%; 50%/50%; 60%/40%; 70%/30%; 75%/25%; 80%/20%; 90%/10%; 95%/5%.

In some embodiments, the at least one collagen comprised in the extracellular matrix is a fibrillar collagen. In some embodiments, said fibrillar collagen is selected from the group consisting of type I collagen, type II collagen, type III collagen, type V collagen, type VI collagen, type XI collagen, type XXIV collagen, type XXVII collagen and any mixtures thereof. In certain embodiments, the collagen, preferably the fibrillar collagen is present in the culture medium in a concentration of from about 0.25 mg/ml to about 3.00 mg/ml. Within the scope of the instant invention, “from about 0.25 mg/ml to about 3.00 mg/ml” encompasses about 0.25 mg/ml, 0.50 mg/ml, 0.75 mg/ml, 1.00 mg/ml, 1.25 mg/ml, 1.50 ng/ml, 1.75 mg/ml, 2.00 mg/ml, 2.25 mg/ml, 2.50 mg/ml, 2.75 mg/ml and 3.00 mg/ml.

In one embodiment, when present, the fibronectin is in a concentration of from about 0.01 mg/ml to about 10 mg/ml. Within the scope of the instant invention, “from about 0.01 mg/ml to about 10 mg/ml” encompasses about 0.01 mg/ml, 0.05 mg/ml, 0.1 mg/ml, 0.25 mg/ml, 0.50 mg/ml, 0.75 mg/ml, 1 mg/ml, 1.25 mg/ml, 1.50 ng/ml, 1.75 mg/ml, 2 mg/ml, 2.25 mg/ml, 2.50 mg/ml, 2.75 mg/ml, 3 mg/ml, 4 mg/ml, 5 mg/ml, 6 mg/ml, 7 mg/ml, 8 mg/ml, 9 mg/ml and 10 mg/ml.

In one embodiment, when present, the gelatin is in a concentration of from about 0.01 mg/ml to about 10 mg/ml. Within the scope of the instant invention, “from about 0.01 mg/ml to about 10 mg/ml” encompasses about 0.01 mg/ml, 0.05 mg/ml, 0.1 mg/ml, 0.25 mg/ml, 0.50 mg/ml, 0.75 mg/ml, 1 mg/ml, 1.25 mg/ml, 1.50 ng/ml, 1.75 mg/ml, 2 mg/ml, 2.25 mg/ml, 2.50 mg/ml, 2.75 mg/ml, 3 mg/ml, 4 mg/ml, 5 mg/ml, 6 mg/ml, 7 mg/ml, 8 mg/ml, 9 mg/ml and 10 mg/ml.

In some embodiments, the method comprises a step c) of freezing the hepatic stem-like cells according to the invention, isolated at step b).

In practice, the hepatic stem-like cells obtained by the methods disclosed herein may be collected, washed, optionally fractionated in order to obtain an extract thereof, and resuspended in a conservation medium, preferably comprising DMSO in a concentration of from about 0.1% (v/v) to about 20% (v/v), more preferably of about 10% (v/v). Alternatively, conservation medium can be free of DMSO, such as PRIME-XV® MSC FreezIS DMSO-Free (IRVINE SCIENTIFIC®), STEM-CELLBANKER® DMSO free (AMSBIO®), Ibidi Freezing Medium DMSO free (IBIDI®), CryoSOfree™ DMSO free Cryopreservation Medium (SIGMA ALDRICH®), trehalose-containing solutions (see, e.g., Ntai et al.; 2018). Illustratively, conservation media may be commercially available (CRYOSTOR®) and be purchased, e.g., from MERCK®.

Within the scope of the instant invention, the expression “from about 0.10% to about 20%” encompasses 0.1%, 0.5%, 1%, 2%, 3%, 4%, 5%, 6%, 7%, 8%, 9%, 10%, 11%, 12%, 13%, 14%, 15, 16%, 17%, 18%, 19%, and 20%.

The hepatic stem-like cells, or an extract thereof, once in a suitable conservation medium may be subjected to a freezing process whereby the final temperature range of from about −80° C. to about −196° C.

In certain embodiments, the hepatic stem-like cells comprised in the population of cells are cryopreserved.

As used, herein, “cryopreserved” and “frozen” may substitute one other.

In another aspect, the invention also relates to cryopreserved hepatic stem-like cells, or an extract thereof, susceptible to be obtained by the method according to the instant invention.

One aspect of the invention relates to a cryopreserved population of cells comprising hepatic stem-like cells, or an extract thereof, according to the invention, in particular, a population susceptible to the obtained by the method according to the invention.

The invention further relates to a cryopreserved in vitro culture of hepatic stem-like cells, or an extract thereof, susceptible to be obtained by the method according to the invention.

Another aspect of the invention relates to a particle, in particular a spheroid, comprising hepatic stem-like cells, or an extract thereof, and/or a population of cells comprising hepatic stem-like cells, or an extract thereof, and/or cells derived from the hepatic stem-like cells, or an extract thereof, according to the instant invention.

In some embodiments, hepatic stem-like cells, the population of cells comprising hepatic stem-like cells, or an extract thereof, according to the invention, is in the form of particles or spheroids.

In certain embodiments, the particle is in the form of a spheroid, preferably a spheroid having a mean diameter comprised from about 50 μm to about 250 μm.

Within the scope of the instant invention, the term “from about 50 μm to about 250 μm” encompasses 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm and 250 μm.

In some embodiments, the particle comprises from about 2 hepatic stem-like cells/particle to about 2,500 hepatic stem-like cells/particle. In certain embodiments, the particle comprises from about 250 hepatic stem-like cells/particle to about 1,500 hepatic stem-like cells/particle. Within the scope of the instant invention, the expression “from 2 to about 2,500 cells/particle” encompasses 2, 4, 6, 8, 10, 12, 14, 16, 18, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1,000, 1,050, 1,100, 1,150, 1,200, 1,250, 1,300, 1,350, 1,400, 1,450, 1,500, 1,550, 1,600, 1,650, 1,700, 1,750, 1,800, 1,850, 1,900, 1,950, 2,000, 2,050, 2,100, 2,150, 2,200, 2,250, 2,300, 2,350, 2,400, 2,450 and 2,500 cells/particle.

In practice, the particles or the spheroids may be obtained by culturing the hepatic stem-like cells, or the population of cells comprising hepatic stem-like cells according to the invention, in a culture medium, optionally supplemented with HGF and/or one or more cytokine and/or one or more ingredient having anti-inflammatory and/or immunosuppressive properties.

In one embodiment, when present in the culture medium, HGF is comprised in a concentration of from about 0.1 ng/ml to about 1,000 ng/ml, preferably from about 1 ng/ml to about 500 ng/ml, more preferably from about 10 ng/ml to about 30 ng/ml. Within the scope of the instant invention, the expression “from about 0.1 ng/ml to about 1,000 ng/ml” encompasses about 0.1 ng/ml, 0.25 ng/ml, 0.5 ng/ml, 0.75 ng/ml, 1 ng/ml, 5 ng/ml, 10 ng/ml, 15 ng/ml, 20 ng/ml, 25 ng/ml, 50 ng/ml, 75 ng/ml, 100 ng/ml, 200 ng/ml, 300 ng/ml, 400 ng/ml, 500 ng/ml, 600 ng/ml, 700 ng/ml, 800 ng/ml, 900 ng/ml and 1,000 ng/ml.

In one embodiment, when present in the culture medium, the cytokine is comprised in a concentration of from about 0.1 ng/ml to about 100 ng/ml, preferably from about 1 ng/ml to about 50 ng/ml, more preferably from about 10 ng/ml to about 30 ng/ml. In one embodiment, the cytokine is oncostatin M (OSM). Within the scope of the instant invention, the expression “from about 0.1 ng/ml to about 100 ng/ml” encompasses about 0.1 ng/ml, 0.25 ng/ml, 0.5 ng/ml, 0.75 ng/ml, 1 ng/ml, 5 ng/ml, 10 ng/ml, 15 ng/ml, 20 ng/ml, 25 ng/ml, 50 ng/ml, 75 ng/ml and 100 ng/ml.

In certain embodiments, the culture medium further comprises an ingredient having anti-inflammatory and/or immunosuppressive properties, preferably a corticosteroid. When present in the culture medium, the corticosteroid is comprised in a concentration of from about 0.01 μM to about 10 μM, preferably from about 0.1 μM to about 5 μM. In one embodiment, the corticosteroid is dexamethasone. Within the scope of the instant invention, the expression “from about 0.01 μM to about 10 μM” encompasses 0.01 μM, 0.025 μM, 0.05 μM, 0.075 μM, 0.1 μM, 0.25 μM, 0.5 μM, 0.75 μM, 1 μM, 1.5 μM, 2 μM, 2.5 μM, 5 μM, 7.5 μM and 10 μM.

In some embodiments, the particle may be obtained after 1 day, 2 days or 3 days in suitable conditions. In practice, the hepatic stem-like cells may be cultured on a solid support (culture vessel) to favorize their aggregation. Illustratively, suitable supports may be commercially available from STEM CELLS TECHNOLOGIES®, such as, e.g., the Aggrewell® plates.

In some embodiments, the particles or the spheroids are prepared from a cryopreserved hepatic stem-like cells, a cryopreserved isolated population of cells comprising hepatic stem-like cells, or an extract thereof, according to the invention.

Without wishing to be bound to a theory, the inventors consider that a cell suspension comprising hepatic stem-like cells assembled into a 3D structure such as spheroids may be more suitable than single cell suspensions for some administration sites, such as, e.g., for intraperitoneal cell transplantation. In some embodiments, the particles or the spheroids according to the invention may represent a bioartificial liver (BAL), suitable to be administered to an individual in need of liver therapy.

Another aspect of the invention relates to particles comprising cells differentiated from the hepatic stem-like cells according to the invention, or a population thereof.

Another aspect of the invention pertains to a suspension comprising hepatic stem-like cells, or an extract thereof, and/or a population of cells comprising hepatic stem-like cells, or an extract thereof, and/or cells derived from the hepatic stem-like cells, or an extract thereof, according to the instant invention.

In some embodiments, the isolated population of cells comprising hepatic stem-like cells, or an extract thereof, according to the invention is in the form of a suspension. As used herein, the term “suspension” refers to a composition wherein the cells or the material are floating cells or material.

In certain embodiments, the suspension may comprise from about 10¹ to about 10¹² hepatic stem-like cells per ml. Within the scope of the instant invention, “from about 10¹ to about 10¹² hepatic stem-like cells per ml” includes 10¹, 5×10¹, 10², 5×10², 10³, 5×10³, 10⁴, 5×10⁴, 10⁵, 5×10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹ and 10¹² hepatic stem-like cells per ml.

In some aspects, the invention relates to a suspension comprising cells differentiated from the hepatic stem-like cells according to the invention, or a population thereof.

The invention further relates to a pharmaceutical composition comprising (i) hepatic stem-like cells, or an extract thereof, and/or a population of cells comprising hepatic stem-like cells, or an extract thereof, and/or cells derived from the hepatic stem-like cells, or an extract thereof, and/or at least one particle, and/or a suspension according to the invention and (ii) a pharmaceutically acceptable vehicle.

As used herein, “pharmaceutically acceptable vehicle” refers to any solvent, dispersion medium, coating, antibacterial and/or antifungal agent, isotonic and absorption delaying agent and the like.

In practice, the pharmaceutically acceptable vehicle may comprise one or more ingredient(s) selected in a group of additives polypeptides; amino acids; lipids; and carbohydrates. Among carbohydrates, one may cite sugars, including monosaccharides, di-, tri-, tetra-, and oligosaccharides; derivatized sugars such as alditols, aldonic acids, esterified sugars and the like; and polysaccharides or sugar polymers.

Exemplary polypeptidic pharmaceutically acceptable vehicle may include gelatin, casein, and the like.

In some embodiments, the pharmaceutical composition may comprise from about 10¹ to about 10¹² hepatic stem-like cells per ml. Within the scope of the instant invention, “from about 10¹ to about 10¹² hepatic stem-like cells per ml” includes 10¹, 5×10¹, 10², 5×10², 10³, 5×10³, 10⁴, 5×10⁴, 10⁵, 5×10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹ and 10¹² hepatic stem cells per ml.

In one aspect, the invention relates to a pharmaceutical composition consists essentially of (i) hepatic stem-like cells, or an extract thereof, and/or a population of cells comprising hepatic stem-like cells, or an extract thereof, and/or cells derived from the hepatic stem-like cells, or an extract thereof, and/or at least one particle, and/or a suspension according to the invention and (ii) a pharmaceutically acceptable vehicle. AS used herein, the term “consists essentially of” is intended to mean that the hepatic stem-like cells, or an extract thereof, and/or a population of cells comprising hepatic stem-like cells, or an extract thereof, and/or cells derived from the hepatic stem-like cells, or an extract thereof, and/or at least one particle, and/or a suspension according to the invention is/are the sole active ingredient of the composition.

In some aspects, the invention relates to a pharmaceutical composition comprising cells differentiated from the hepatic stem-like cells according to the invention, or a population thereof, and a pharmaceutically acceptable vehicle.

Another aspect of the invention relates to a medical device comprising hepatic stem-like cells, or an extract thereof, and/or a population of cells comprising hepatic stem-like cells, or an extract thereof, and/or cells derived from the hepatic stem-like cells, or an extract thereof, and/or at least one particle, and/or a suspension, and/or a pharmaceutical composition according to the invention.

In certain embodiments, the medical device comprises one or more item selected in the group consisting of a pump, filter, tubing, catheter, and the like.

In some embodiments, the medical device is in the form of an external bioartificial liver (EBAL). In some embodiments, the medical device, in particular the external bioartificial liver, comprises a bioreactor comprising hepatic stem-like cells, and/or the population of cells according to the instant invention and/or cells derived from the hepatic stem-like cells. In certain embodiments, the medical device, in particular the external bioartificial liver, may further comprise at least one heparin pump, at least one plasma filter, at least one roller pump.

In practice, the medical device, in particular the external bioartificial liver, may comprise a unit that resembles a cardio-pulmonary bypass machine. In practice, the medical device, in particular the external bioartificial liver, is configured to treat the patient's blood plasma before being returned to the patient.

Non-limitative examples of suitable medical devices for implementing the invention are described in Struecker et al. (2014); Glorioso et al. (2015); Chen et al. (2019).

In some embodiments, the medical device according to the invention is used for extracorporeal liver therapy.

Another aspect of the invention relates to a non-human animal model comprising heterologous hepatic stem-like cells, or an extract thereof, and/or a heterologous population of cells comprising hepatic stem-like cells, or an extract thereof, and/or heterologous cells derived from the hepatic stem-like cells, or an extract thereof.

As used herein, the term “heterologous” is intended to mean that the non-human animal and the cells are not originating from the same species.

In some embodiments, the non-human animal model is a humanized non-human animal model. As used herein, the term “humanized” is intended to mean that the non-human animal model comprises human hepatic stem-like cells, human cells derived from the hepatic stem-like cells, or an extract thereof, according to the instant invention.

In some embodiments, the non-human animal is a non-human mammal, preferably selected in the group consisting of dogs, cats, guinea pigs, rats, mice, rabbits, cattle, sheep, goats, horses, llamas, monkeys. In certain embodiments, the non-human animal is a mouse or a rat.

In practice, the non-human animal model is administered with the hepatic stem-like cells, and/or the population of cells, and/or cells derived from the hepatic stem-like cells, and/or an extract thereof, as disclosed by the invention, so that, the liver of the animal comprises heterologous hepatic stem like cells, and/or heterologous cells derived from the hepatic stem-like cells, and/or an extract thereof. In some embodiments, the non-human animal model may be used to assess the liver toxicity of a drug candidate. As used herein, the term “liver toxicity” is intended to refer to a degree of being poisonous towards the liver. By extension, the term “liver toxicity of a drug candidate” is intended to refer to the degree by which the drug candidate limits, restrains, inhibits, precludes or prevents the liver to exert its natural and physiological detoxifying function, as compared to a healthy functional liver.

The drug candidate may be evaluated through the assessment of its impact onto monitored biological parameters, such as, e.g., temperature, weight gain or weight loss, respiratory capacity, encephalogram, cardiogram, cognitive capacity, mortice capacity, level of serum markers, blood numeration, and the likes.

In some embodiments, the non-human animal model may be treated with a compound suitable to generate a liver disorder. In said embodiments, the non-human animal model with a liver disorder may be used to assess the efficacy of drug candidates intended to treat or prevent said liver disorder, in particular to promote apoptosis of the diseased cells and/or to repair the diseased cells into non-diseased, particularly normal cells and/or to stimulate the proliferation of non-diseased cells. Non-limitative examples of compounds suitable to generate a liver disorder include acetaminophen (APAP), alcohol, aspirin, ibuprofen, naproxen sodium and thioacetamide. In some embodiments, the non-human animal model may be infected with an infectious agent, such as a pathogenic bacterium and/or a virus.

Another aspect of the invention relates to hepatic stem-like cells, or an extract thereof, or the population of cells comprising hepatic stem-like cells, or an extract thereof, or cells derived from the hepatic stem-like cells, or an extract thereof, or the particle, or the suspension, or the pharmaceutical composition, according to the instant invention, for use as a medicament.

The invention further relates to the use of hepatic stem-like cells, or a population of cells comprising hepatic stem-like cells, or cells derived from the hepatic stem-like cells, or an extract thereof, or a particle, or a suspension, or a pharmaceutical composition, according to the instant invention for the preparation or the manufacture of a medicament.

Another aspect of the invention relates to hepatic stem-like cells, or an extract thereof, or the population of cells comprising hepatic stem-like cells, or an extract thereof, or cells derived from the hepatic stem-like cells, or an extract thereof, or the particle, or the suspension, or the pharmaceutical composition, or the medical device according to the instant invention, for use for treating and/or preventing a liver disorder.

A further aspect of the invention relates to the use of hepatic stem-like cells, or an extract thereof, or the population of cells comprising hepatic stem-like cells, or an extract thereof, or cells derived from the hepatic stem-like cells, or an extract thereof, or the particle, or the suspension, or the pharmaceutical composition, or the medical device according to the instant invention, for treating and/or preventing a liver disorder.

The invention further relates to a method for treating and/or preventing a liver disorder in an individual in need thereof, comprising the administration of a therapeutically efficient amount of hepatic stem-like cells, and/or the population, or cells derived from the hepatic stem-like cells, or an extract thereof, or the particle, or the suspension, or the pharmaceutical composition according to the instant invention.

The invention further relates to a method for treating and/or preventing a liver disorder in an individual in need thereof, comprising the step of implementing a medical device according to the instant invention. The invention further relates to a method for treating and/or preventing a liver disorder in an individual in need thereof, comprising the steps of:
a) connecting the individual to a medical device according to the instant invention, by the mean of an extracorporeal blood circuit;
b) providing the medical device with the plasma of said individual, so that the plasma is detoxified by the medical device; and
c) providing the individual with the detoxified plasma generated at step b).

In another aspect, the invention relates to the use of a medical device according to the invention in a method for treating and/or preventing a liver disorder in an individual in need thereof.

In certain embodiments, the liver disorder is selected in the group consisting of Alagille syndrome; alcohol-related liver disease; alpha-1 antitrypsin deficiency; autoimmune hepatitis; benign liver tumors; biliary atresia; cirrhosis; hemochromatosis; hepatic encephalopathy; hepatitis A; hepatitis B; hepatitis C; hepatorenal Syndrome; intrahepatic cholestasis of pregnancy (ICP); lysosomal acid lipase deficiency (LAL-D); liver cysts; liver cancer; newborn jaundice; non-alcoholic fatty liver disease; non-alcoholic steatohepatitis; primary biliary cholangitis (PBC); primary sclerosing cholangitis (PSC); progressive familial intrahepatic cholestasis (PFIC); Reye syndrome; type I glycogen storage disease; an acute liver failure (ALF); an acute chronic liver failure (ACLF); the non-alcoholic steato-hepatitis (NASH); alcoholic hepatitis; viral-induced hepatitis; a cryptogenic liver disease; a malignant liver disease, such as, e.g., hepatocellular carcinoma and cholangiocarcinoma; autoimmune hepatitis, a vascular liver disease, such as, e.g., Budd-Chiari syndrome; a cholestatic liver disease; an inherited metabolic liver disease, such as, e.g., Wilson's disease and an urea cycle disorder.

In certain embodiments, the liver disorder is a fulminant liver disorder.

As mentioned above, a fulminant liver disorder refers to any disorder prioritized for liver transplantation using a scoring system for organ allocation such as the Model for End-stage Liver Disease (MELD).

Another aspect of the invention relates to hepatic stem-like cells, or an extract thereof, or the population of cells comprising hepatic stem-like cells, or an extract thereof, or cells derived from the hepatic stem-like cells, or an extract thereof, or the particle, or the suspension, or the pharmaceutical composition, or the medical device according to the instant invention, for use for treating and/or preventing a fulminant liver disorder.

A further aspect of the invention relates to the use of hepatic stem-like cells, or an extract thereof, or the population of cells comprising hepatic stem-like cells, or an extract thereof, or cells derived from the hepatic stem-like cells, or an extract thereof, or the particle, or the suspension, or the pharmaceutical composition, or the medical device according to the instant invention, for treating and/or preventing a fulminant liver disorder.

The invention further relates to a method for treating and/or preventing a fulminant liver disorder in an individual in need thereof, comprising the administration of a therapeutically efficient amount of the hepatic stem-like cells, or the population, or cells derived from the hepatic stem-like cells, or an extract thereof, or the particle, or the suspension, or the pharmaceutical composition, according to the instant invention.

In some embodiments, said fulminant liver disorder is selected in the group consisting of an acute liver failure (ALF) and an acute chronic liver failure (ACLF).

In certain embodiments, the acute chronic liver failure (ACLF) may be associated with a liver disease, in particular a chronic liver disease, selected group consisting of the non-alcoholic steato-hepatitis (NASH); alcoholic hepatitis; viral-induced hepatitis; a cryptogenic liver disease; a malignant liver disease, such as, hepatocellular carcinoma and cholangiocarcinoma; autoimmune hepatitis, a vascular liver disease, such as, Budd-Chiari syndrome; a cholestatic liver disease; and an inherited metabolic liver disease, such as, Wilson's disease and an urea cycle disorder. In other words, in certain embodiments, the ACLF refers to a highly specific and rare syndrome, characterized by an acute abnormality of liver blood tests in an individual with underlying chronic liver disease, in particular selected group consisting of the non-alcoholic steato-hepatitis (NASH); alcoholic hepatitis; viral-induced hepatitis; a cryptogenic liver disease; a malignant liver disease, such as, hepatocellular carcinoma and cholangiocarcinoma; autoimmune hepatitis, a vascular liver disease, such as, Budd-Chiari syndrome; a cholestatic liver disease; and an inherited metabolic liver disease, such as, Wilson's disease and an urea cycle disorder.

In some embodiments, said fulminant liver disorder is an acute liver failure (ALF) or an acute chronic liver failure (ACLF).

In certain embodiments, the acute chronic liver failure (ACLF) is associated with a liver disease selected group consisting of; the non-alcoholic steato-hepatitis (NASH); alcoholic hepatitis; viral-induced hepatitis; a cryptogenic liver disease; a malignant liver disease, such as, hepatocellular carcinoma and cholangiocarcinoma; autoimmune hepatitis, a vascular liver disease, such as, Budd-Chiari syndrome; a cholestatic liver disease; and an inherited metabolic liver disease, such as, Wilson's disease and an urea cycle disorder.

In some embodiments, the fulminant liver disorder is an acute liver failure (ALF).

In certain embodiments, the fulminant liver disorder is an acute chronic liver failure (ACLF).

Without wanting to be bound to a theory, the inventors consider that the liver regeneration properties of the hepatic stem-like cells according to the invention towards ALF and ACLF are driven by the action of the cells towards the healthy liver cells within the diseased liver, by promoting their proliferation. In other words, the liver regeneration properties are mediated by mainly promoting the regeneration of the healthy liver tissue of a diseased liver rather than being mediated by the replacing the diseased cells.

The invention further relates to a method for regenerating a liver in an individual with liver disorder, in particular a fulminant liver disorder, comprising the step of administering to said individual a therapeutically efficient amount of the hepatic stem-like cells, or an extract thereof, the population of cells, or an extract thereof, or the cells derived from the hepatic stem-like cells, or an extract thereof, or the particle, or the suspension, or the pharmaceutical composition according to the instant invention.

A still other aspect of the invention also relates to a method for decreasing the levels of alanine aminotransferase (ALAT) in the serum of an individual with a liver disorder, in particular a fulminant liver disorder, comprising the step of administering to said individual a therapeutically efficient amount of the hepatic stem-like cells, or an extract thereof, the population of cells, or an extract thereof, or the cells derived from the hepatic stem-like cells, or an extract thereof, or the particle, or the suspension, or the pharmaceutical composition according to the instant invention.

Another aspect of the invention further pertains to a method for decreasing liver necrosis in an individual with a liver disorder, in particular a fulminant liver disorder, comprising the step of administering to said individual a therapeutically efficient amount of the hepatic stem-like cells, or an extract thereof, the population of cells, or an extract thereof, or the cells derived from the hepatic stem-like cells, or an extract thereof, or the particle, or the suspension, or the pharmaceutical composition according to the instant invention.

In practice, the individual with a liver disorder, in particular a fulminant liver disorder, may be diagnosed by the mean of a clinical examination and/or blood tests and/or a liver biopsy, following the good practice and the standards in the field.

One aspect of the invention relates to the use of a cryopreserved population of cells comprising hepatic stem-like cells, or an extract thereof, according to invention, for preparing a particle, as defined in the instant disclosure.

In some embodiments, the particle is in the form of a spheroid.

In certain embodiments, the therapeutically efficient amount of the hepatic stem-like cells, and/or the population of cells comprising hepatic stem-like cells, and/or the cells derived from the hepatic stem-like cells, and/or an extract thereof, and/or the particle, and/or the suspension and/or the pharmaceutical composition according to the invention, to be administered may easily be determined by a skilled and/or authorized personnel.

In practice, the therapeutically efficient amount may depend upon a variety of parameters, including the material selected for administration, whether the administration is in single or multiple doses, and the individual's parameters including age, physical conditions, size, weight, gender, and the severity of the fulminant liver disorder to be treated.

In some embodiments, the therapeutic efficient amount is from about 10¹ to about 10¹² hepatic stem-like cells per ml. In practice, a therapeutic efficient amount includes 10¹, 5×10¹, 10², 5×10², 10³, 5×10³, 10⁴, 5×10⁴, 10⁵, 5×10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹ and 10¹² hepatic stem-like cells per ml. In certain embodiments, the therapeutically efficient amount is from about 10¹ to about 10¹² hepatic stem cells per cm³, which includes 10¹, 5×10¹, 10², 5×10², 10³, 5×10³, 10⁴, 5×10⁴, 10⁵, 5×10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹ and 10¹² hepatic stem-like cells per cm³. In some embodiments, the therapeutically efficient amount is from about 10¹ to about 10¹² hepatic stem cells per dose, which includes 10¹, 5×10¹, 10², 5×10², 10³, 5×10³, 10⁴, 5×10⁴, 10⁵, 5×10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹ and 10¹² hepatic stem-like cells per dose.

For therapy, hepatic stem-like cells, populations of cells comprising hepatic stem-like cells, particles, suspensions and pharmaceutical compositions according to the invention may be administered through different routes. The dose and the number of administrations can be optimized by those skilled in the art in a known manner.

In some embodiments, the hepatic stem-like cells, the population of cells comprising hepatic stem-like cells, the cells derived from the hepatic stem-like cells, the extract thereof, the particles, the suspension or the pharmaceutical composition of the invention is to be administered locally or systemically.

In some embodiments, the hepatic stem-like cells, the population of cells comprising hepatic stem-like cells, the cells derived from the hepatic stem-like cells, the extract thereof, the particles, the suspension or the pharmaceutical composition of the invention is/are to be administered locally and include without limitation, an injection or an infusion or an implantation of the population of cells comprising hepatic stem-like cells, particles, suspension or pharmaceutical composition of the invention in, around or near the liver, in the liver parenchyma, under the liver Glisson's capsule, under kidney capsule, in the spleen, in the pancreas, in the peritoneum and omental pouch. Preferably, the local administration is an injection or an infusion or an implantation via blood vessels irrigating the liver (portal vein, artery, vein, mesenteric veins).

In some embodiments, the hepatic stem-like cells, the population of cells comprising hepatic stem-like cells, the cells derived from the hepatic stem-like cells, the extract thereof, the particles, the suspension or the pharmaceutical composition of the invention is/are to be administered via an intraperitoneal, an intravenous, an intraportal or an intrasplenic administration, in particular via an intraperitoneal, an intravenous, an intraportal or an intrasplenic injection.

In another embodiment, the hepatic stem-like cells, the population of cells comprising hepatic stem-like cells, the cells derived from the hepatic stem-like cells, the extract thereof, the particles, the suspension or the pharmaceutical composition of the invention is/are to be administered in a differentiating environment for the population of human hepatic stem-like cells of the invention.

Such route of administration can be achieved by surgery procedure, laparoscopic surgery, via a catheter system or an implantation in the peritoneal cavity.

In some embodiments, the hepatic stem-like cells, the population of cells comprising hepatic stem-like cells, the cells derived from the hepatic stem-like cells, the extract thereof, the particles, the suspension or the pharmaceutical composition of the invention is/are to be administered systemically and include without limitation, intraperitoneal, subcutaneous, enteral or parenteral administration.

Examples of formulations adapted to injection or infusion or implantation include, but are not limited to, liquid solutions or suspensions, solid forms suitable for solution in, or suspension in, liquid prior to injection. Examples of injections include, but are not limited to, intraportal, intrasplenic, intravenous, intra-aortic, intraperitoneal, subcutaneous, intramuscular, intradermal, and intraperitoneal injection, or perfusion. In some embodiments, when injected, the hepatic stem-like cells, the population of cells comprising hepatic stem-like cells, the cells derived from the hepatic stem-like cells, the extract thereof, the particles, the suspension or the pharmaceutical composition of the invention is/are sterile. Methods for obtaining a sterile pharmaceutical composition include, but are not limited to, GMP synthesis (GMP stands for “Good manufacturing practice”).

In some embodiments, the hepatic stem-like cells, the population of cells comprising hepatic stem-like cells, the cells derived from the hepatic stem-like cells, the extract thereof, the particles, the suspension or the pharmaceutical composition of the invention is/are encapsulated. Examples of capsules include without limitation, Matrigel®, biocompatible hydrogels. Methods for encapsulating biological active principles in hydrogels are known from a skilled in the art. One can refer to Perez-Luna et al. (2018).

As used herein, “hydrogel” is intended to refer to a hydrophilic, three-dimensional network, which is capable of uptaking large amounts of water or biological fluids and where the cells are entrapped. In practice, the network comprises homopolymers or copolymers, and is insoluble. Suitable polymers for constituting the network include, without to be limited to, sodium alginate, acrylic acid, cellulose sulphate, ethylene glycol, ethylene glycol dimethacrylate (EGDMA), hyaluronic acid, hydroxyethyl methacrylate (HEMA), hydroxyethoxyethyl methacrylate (HEEMA), hydroxydiethoxyethyl methacrylate (HDEEMA), methoxyethyl methacrylate (MEMA), N-vinyl-2-pyrrolidone (NVP), PEG acrylate (PEGA), PEG methacrylate (PEGMA), PEG diacrylate (PEGDA), PEG dimethacrylate (PEGDMA), silanized hydroxypropyl methyl cellulose (si-HPMC) and the likes.

Hydrogels are particularly disclosed in Peppas et al. (2000); Narayanaswamy and Torchilin (2019).

In some embodiments, the encapsulated hepatic stem-like cells, population of cells comprising hepatic stem-like cells, cells derived from the hepatic stem-like cells, extract thereof, particles, suspension or pharmaceutical composition may be administered by any route, including by intraperitoneal, intravenous, intraportal, intra-tissular injection or any other suitable mode of injection.

In practice, the hydrogel may serve to concentrate the hepatic stem-like cells, the population of cells comprising hepatic stem-like cells, the cells derived from the hepatic stem-like cells, the extract thereof, the particles, the suspension or the pharmaceutical composition of the invention. In certain embodiments, the hydrogel may be incorporated in a patch, in particular, a patch for the sustained release and/or functionality of the hepatic stem-like cells, the population of cells comprising hepatic stem-like cells, the cells derived from the hepatic stem-like cells, the extract thereof, the particles, the suspension or the pharmaceutical composition according to the invention.

One aspect of the invention relates to a patch comprising the hepatic stem-like cells, the population of cells comprising hepatic stem-like cells, the cells derived from the hepatic stem-like cells, the extract thereof, the particles, the suspension or the pharmaceutical composition according to the invention, and optionally a hydrogel.

In practice, the patch may be locally administered on the liver, or on any organs.

In some embodiments, the hepatic stem-like cells, the population of cells comprising hepatic stem-like cells, the cells derived from the hepatic stem-like cells, the extract thereof, the particles, the suspension or the pharmaceutical composition of the invention is/are to be administered in a sustained-release form. In another embodiment, the hepatic stem-like cells, the population of cells comprising hepatic stem-like cells, cells derived from the hepatic stem-like cells, the extract thereof, the particles, the suspension or the pharmaceutical composition of the invention is/are formulated as a delivery system that controls the release of the agent.

In some embodiments, a therapeutically effective amount of the hepatic stem-like cells, the population of cells comprising hepatic stem-like cells, cells derived from the hepatic stem-like cells, the extract thereof, the particles, suspension or pharmaceutical composition of the invention is/are to be administered at least once in the subject's life or several times to obtain and/or to maintain therapeutic benefit in the subject.

In practice, the administration of the hepatic stem-like cells, population of cells comprising hepatic stem-like cells, or an extract thereof, particles, suspension or pharmaceutical composition of the invention may be considered as a graft or a transplant.

Illustratively, the administration of the hepatic stem-like cells, population of cells comprising hepatic stem-like cells, or an extract thereof, particles, suspension or pharmaceutical composition according to the invention may be referred to as grafting or transplantation.

In certain embodiments, the transplantation is autologous. In this peculiar embodiment, the cells used for preparing the hepatic stem-like cells, the population of cells comprising hepatic stem-like cells, or the cells derived from the hepatic stem-like cells, or an extract thereof, according to the invention are originating from the same individual than the individual receiving the transplantation.

In alternative embodiments, the transplantation is allogenic. In this particular embodiment, the cells used for preparing the hepatic stem-like cells, the population of cells comprising hepatic stem-like cells, or the cells derived from the hepatic stem-like cells, or an extract thereof, according to the invention are originating from an individual from the same species but distinct from the individual receiving the transplantation.

In some embodiments, the individual with a liver disorder, in particular with a fulminant liver disorder, may undergo a surgery prior to the administration of the hepatic stem-like cells, the population of cells comprising hepatic stem-like cells, the cells derived from the hepatic stem-like cells, or an extract thereof, the particles, the suspension or the pharmaceutical composition according to the invention, as for removing at least part of the diseased liver tissue, in particular the necrosed liver tissue.

In certain embodiments, the hepatic stem-like cells, the population of cells comprising hepatic stem-like cells, the cells derived from the hepatic stem-like cells, or an extract thereof, the particles, the suspension or the pharmaceutical composition according to the invention may be co-administered with one or more additional active agents, intended to promote or favorize liver regeneration. As used herein, the term “co-administered” includes a simultaneous administration and a sequential administration.

The invention also relates to a combination product, which comprises:
at least one hepatic stem-like cell, a population of cells comprising hepatic stem-like cells, and/or an extract thereof, and/or particles, and/or suspension and/or pharmaceutical composition according to the invention; and
at least one additional active agent, in particular intended to favorize liver regeneration;
for simultaneous, separate or sequential administration.

As used herein, “favorize liver regeneration” refers to the cessation or the lowering of the degradation of the liver tissue, and encompasses the partial or complete recovery of the physiological functions of a healthy liver tissue. Non limitative examples of suitable additional active agent may be an anti-inflammatory agent, an immunosuppressive agent, an antibiotic, an anti-oxidant, an antifibrotic agent, a detoxifying agent.

Non-limitative examples of anti-inflammatory agents include aspirin, celecoxib, diclofenac, etoricoxib, ibuprofen, indomethacin, mefenamic acid and naproxen.

Non-limitative examples of immunosuppressive agents include calcineurin inhibitors, such as, e.g., cyclosporine, tacrolimus; interleukin inhibitors, such as, e.g., basiliximab, benralizumab, brodalumab, daclizumab, dupilumab, ixekizumab, mepolizumab, sarilumab, tocilizumab; TNF alfa inhibitors, such as, e.g., adalimumab, etanercept, golimumab and infliximab.

Non-limitative examples of antibiotics include penicillins, such as, e.g., ampicillin, amoxicillin and dicloxacillin; tetracyclines such as, e.g., demeclocycline, doxycycline, eravacycline, minocycline, omadacycline and tetracycline; cephalosporins, such as, e.g., cefaclor, cefdinir, cefotaxime, ceftazidime, ceftriaxone, and cefuroxime; quinolones such as, e.g., ciprofloxacin, levofloxacin and moxifloxacin; lincomycins, such as, e.g., clindamycin and lincomycin; macrolides such as, e.g., azithromycin, clarithromycin and erythromycin; sulfonamides such as, e.g., sulfasalazine, sulfamethoxazole and trimethoprim; glycopeptides such as, e.g., dalbavancin, oritavancin, telavancin and vancomycin; aminoglycosides such as, e.g., amikacin, gentamicin and tobramycin; carbapenems such as, e.g., doripenem, ertapenem and meropenem.

Non-limitative examples of anti-oxidants include carotenoids, such as, e.g., beta-carotene, lycopene, lutein, and zeaxanthin; selenium; vitamin C and vitamin E.

Non-limitative examples of antifibrotic agents include nintedanib and pirfenidone.

Non-limitative examples of detoxifying agents include N-acetyl cysteine.

In some embodiments, the additional active agent may be administered before, during or after the administration of the hepatic stem-like cells, the population of hepatic stem-like cells, the cells derived from the hepatic stem-like cells, or an extract thereof, the particles, the suspension or the pharmaceutical composition according to the invention.

In certain embodiments, the hepatic stem-like cells, the population of cells comprising hepatic stem-like cells, the cells derived from the hepatic stem-like cells, or an extract thereof, the particles, the suspension or the pharmaceutical composition according to the invention is/are not co-administered with an immunosuppressive agent. As illustrated by the examples section below, survival of ALF-mice following a treatment with the hepatic stem-like cells according to the invention could be observed irrespective of whether an immunosuppressive agent was co-administered or not. Advantageously, the treatment with the hepatic stem-like cells according to the invention may be envisioned as a basis for allogenic (heterologous) graft therapy.

Another aspect of the invention pertains to an in vitro method for screening a drug candidate, said method comprising the steps of:
a) providing at least one hepatic stem-like cell, or an extract thereof, and/or a population of cells comprising hepatic stem-like cells, or an extract thereof, and/or cells derived from the hepatic stem-like cells, or an extract thereof, and/or a particle, and/or a suspension, according to the invention;
b) contacting said at least one cell or an extract thereof, and/or said population of cells and/or extract thereof, said cells derived from the hepatic stem-like cells, or an extract thereof, and/or said particle, and/or said suspension, from step a), with a drug candidate;
c) measuring one or more biological parameter(s) and optionally comparing said one or more biological parameter(s) with one or more reference parameter(s);
d) determining whether the drug candidate is of therapeutic and/or diagnostic interest.

As used herein, the term “drug candidate” refers to a compound with potential therapeutic property and/or commercial interest. Within the scope of the instant invention, a drug candidate may have analgesic properties, antibiotic properties, anticancer properties, anticoagulant properties, anti-diuretic properties, anti-inflammatory properties, antiviral properties, hemostatic properties, neuroleptic properties, proliferative activity, anti-fibrotic, anti-steatosis activity, anti-oxidative stress and the likes. In some embodiments, the drug candidate has liver regenerative properties.

In some embodiments, the cells derived from the hepatic stem-like cells, in particular the hepatocyte like cells or the population of cells comprising hepatocyte like cells (HLCs) derived from a population of cells comprising hepatic stem-like cells according to the invention, are expressing the ALB marker (ALB+) and/or the CYP3A4 marker (CYP3A4+), preferably are expressing both the ALB marker (ALB+) and the CYP3A4 marker (CYP3A4+). In some embodiments, the hepatocyte like cells (HLCs) derived from a population of cells comprising hepatic stem-like cells according to the invention are not expressing the AFP marker (AFP−). In certain embodiments, the HLCs are human HLCs derived from the hepatic stem-like cells according to the invention.

In certain embodiments, step a) may be performed by incubating the hepatic stem-like cells, or the population of cells comprising the hepatic stem-like cells according to the invention with a HLC differentiation culture medium. Suitable culture media are similar to the culture media described to generate the hepatic stem-like cells according to the invention, such as, e.g., RPMI medium. In some embodiments, the HLC differentiation culture medium may comprise a compound selected in a group consisting of dexamethasone, a FGF (fibroblast growth factor), FSK (also referred to as the 6-[(1Z)-3-fluoro-2-(hydroxymethyl)prop-1-en-1-yl]-1,5-dimethylpyrimidine-2,4(1H,3H)-dione), a HGF (hepatocyte growth factor), a KGF (keratin growth factor), a GSK3 inhibitor (such as, e.g., CHIR-99021), oncostatin M, a TGF/Smad inhibitor (such as, e.g., SB431542), a notch inhibitor.

In some embodiments, the biological parameter is selected in the group consisting of a proliferative state of the cells, an apoptotic state of the cells, a necrosis state of the cells, a level of an enzyme (such as, e.g., ALAT, ASAT) or a compound of interest (such as, e.g., interleukin, cytokine) in the serum, the level of a biomarker of interest, and the likes. In practice, the biological parameter may be measured at the nucleic acid level, in particular at the mRNA levels, such as, e.g., by RT-PCR; or at the polypeptide or protein level, such as, e.g., by an immunofluorescence, FACS, ELISA, an enzymatic assay and the likes.

In some embodiments, the reference parameter may originate from a healthy individual, in particular is a mean value for said parameter. In some embodiments, the reference parameter may originate from a placebo, assayed following identical conditions as compared to the drug candidate.

In some embodiments, the in vitro method for screening a drug candidate according to the invention may be used to assess the liver toxicity of said drug candidate and/or for drug screening. In some embodiments, the in vitro method for screening a drug candidate according to the invention may be used to assess the ability of said drug candidate to affect the liver, in particular to heal or regenerate a diseased liver.

The invention also pertains to a kit for treating and/or preventing a fulminant liver disorder, said kit comprising:
a) at least one hepatic stem-like cell, or an extract thereof, or population of cells comprising hepatic stem-like cells, or an extract thereof, or cell derived from the hepatic stem-like cell, or an extract thereof, or particle, or suspension or pharmaceutical composition; and
b) a mean to administer said cell, population, cell derived from said hepatic stem-like cell, extract thereof, particle, suspension or pharmaceutical composition.

In some embodiment, the liver disorder is a fulminant liver disorder.

In certain embodiments, said fulminant liver disorder is selected in a group consisting of an acute liver failure (ALF) and acute chronic liver failure (ACLF); wherein the ACLF is associated with a liver disease selected in the group consisting of the non-alcoholic steato-hepatitis (NASH); alcoholic hepatitis; viral-induced hepatitis; a cryptogenic liver disease; a malignant liver disease, such as, hepatocellular carcinoma and cholangiocarcinoma; autoimmune hepatitis, a vascular liver disease, such as, Budd-Chiari syndrome; a cholestatic liver disease; an inherited metabolic liver disease, such as, Wilson's disease and an urea cycle disorder.

In some embodiments, the pharmaceutical composition comprises from about 10¹ to about 10¹² hepatic stem-like cells per ml.

In certain embodiments, the pharmaceutical composition comprises from about 10¹ to about 10¹² hepatic stem-like cells per cm³. In some embodiments, the mean to administer the said cells or said population is a syringe or a catheter. In some embodiments, the kit further comprises one or more additional active agent(s), in particular selected in a group comprising an anti-inflammatory agent, an immunosuppressive agent, an antibiotic, and a mixture thereof. It is understood that the additional active agent is intended to favorize liver regeneration.

In some embodiments, the kit may be of use for performing cell implantation (intracorporeal therapy) or alternatively for performing an extracorporeal liver therapy.

In some embodiments, the kit according to the invention may be of use to generate a medical device, in particular an external bioartificial liver (EBAL), as disclosed herein.

### BRIEF DESCRIPTION OF THE DRAWINGS

FIGS. 1A-D are histograms showing the relative levels of mRNAs of markers, determined by RT-qPCR, encoding pluripotency (OCT4) (Panel A), definitive endoderm (SOX17) (Panel B)), and hepatic progenitor genes (HNF4A) (Panel C), AFP (Panel D) at day 11 (pStemHeps). The relative gene expression was calculated using the 2-ΔΔCt quantification method after normalization to GAPDH values and expressed as fold of levels found in undifferentiated hESCs cells (D0). Relative gene expression levels are in Log 10 scale for SOX17 (Panel B), HNF4A (Panel C) and AFP (Panel D). * P<0.05.

FIGS. 2A-D are photographs and plots showing the expression or the non-expression of different key markers through hepatic differentiation of hESCs from day 0 (D0), day 5 (D5), day 11 (D11) by immunofluorescence assays (OCT4, FOXA2, AFP and ALB) (Panel A), by flow cytometry for SOX17/HNF4A (Panel B) and for CXCR4 (Panel C) and by ELISA measuring AFP secretion in cell supernatant (Panel D). HPH: human primary hepatocytes, * P<0.05.

FIGS. 3A-C are photographs and graph showing the expression of ALB, AFP and HNF4A by immunofluorescence test (Panel A) at D21 after hepatic differentiation of hESC into HLCs, cell morphology of pStemHeps and HLCs examined by phase contrast microscopy (Panel B) and secretion of ALB in cell supernatant by HLCs measured by ELISA test (Panel C). * and ** P<0.05.

FIG. 4 is a plot showing the percentage survival of mice that did not received (plot 1, control animals with no APAP, n=10) or received (plots 2 and 3) 400 mg/kg body weight of acetaminophen (APAP) and further received (plot 2, APAP+pStemHeps, n=10) or not (plot 3, control ALF animals with APAP only, n=10) transplantation of 1×10⁶ frozen pStemHeps that were prepared accordingly to the protocol A, as described in Example 1.

FIG. 5 is a plot showing the alanine aminotransferase (ALAT) levels (expressed in U/L) in the blood of mice that did not received APAP (NO APAP only, n=5), received only APAP (APAP only, n=6) or received APAP and 1×10⁶ frozen pStemHeps (APAP+pStemHeps, n=8), treated as in FIG. 4. Serum ALAT was measured at 24 hours post-cell injection. * and ** P<0.05.

FIG. 6 is a plot showing the percentage of healthy tissue in the three groups of mice treated as in FIG. 4 (APAP+pStemHeps, n=10; APAP only, n=8; No APAP, n=5) and at 24 hours post-cell injection. Each dot corresponds to an animal. * and ** P<0.05.

FIG. 7 is a plot showing the levels of production of human AFP in animal serum measured by ELISA in animals receiving only APAP (APAP only, n=3) and receiving APAP and pStemHeps (APAP+pStemHeps, n=9), treated as in FIG. 4. Human AFP levels were determined at 24 h post-transplantation of 1×10⁶ frozen pStemHeps. Each dot corresponds to an animal. * P<0.05.

FIG. 8 is a plot showing the presence of human Alu sequences in liver biopsies collected from the three groups of mice treated as in FIG. 4 and at 24 h after transplantation of 1×10⁶ frozen pStemHeps as measured by PCR analysis and PCR amplicons detection by capillary electrophoresis. The arrow corresponds to the Alu PCR amplicon. Lanes 1-4: APAP+pStemHeps; lanes 5-6: APAP only; lanes 7-8: No APAP, no cells; lane 9: CTL+(liver of a mice transplanted with human cells); lane 10: Blank; lane 11: ladder.

FIG. 9 is a histogram showing the relative levels of mRNAs of characteristic cell markers, determined by RT-qPCR in pStemHeps that were prepared accordingly to the protocol A, B or C as described in Example 2. The relative gene expression levels were expressed as fold of those found in undifferentiated hESCs. They are significantly different from hESCs. Relative gene expression levels are in Log 10 scale.

FIG. 10 is a histogram showing the expression or non-expression of mRNAs for some characteristic cell markers, determined by DGE-Seq by pStemHeps that were prepared accordingly to the protocol A (n=11; dark grey bars) or protocol B (n=3; light grey bars) as described in Example 2. Depicted expressed genes are defined as genes for which the number of mRNA are above 200 molecules per million of total mRNA molecules (highly expression, set 1), and genes for which the number of mRNA is between 5 and 200 molecules per million of total mRNA molecules and the fold induction change versus undifferentiated hESCs (n=14) is above 5 (lower expressed genes as compared to set 1). Depicted non expressed gene are defined as genes for which the number of mRNA is zero or below 5 molecules per million of total mRNA molecules.

FIGS. 11A-B are a plot and photographs showing the expression of AFP and non-expression of ALB by pStemHeps that were prepared accordingly to the protocol A, B or C by ELISA test (Panel A) and the expression of AFP, CK19, EPCAM, FOXA2, HNF4A, K167, SOX17 and non-expression of ALB by immunofluorescence test (Panel B), by pStemHeps that were prepared accordingly to the protocol C, as described in Example 2.

FIG. 12 is a plot showing the percentage survival of mice (n=10 in each group) as in FIG. 4, except that frozen pStemHeps were prepared accordingly to protocol B, as described in Table 2 of Example 2. Plot 1: CTRL NO APAP, Plots 2: APAP+pStemHeps, plot 3: APAP only.

FIGS. 13A-B are plots showing the percentage survival of mice as in FIG. 12, except that mice have undergone surgery to remove ⅓ of the liver just before transplantation of pStemHeps that were prepared accordingly to protocol B (Panel A) of protocol C (Panel B), as described in Table 2 of Example 2. Panel A: plot 1: APAP+pStemHeps (n=5); plot 2: APAP only (n=10); Panel B: plot 1: APAP+pStemHeps (n=5); plot 2: APAP only (n=5).

FIG. 14A-C is a set of plot and photographs showing (Panel A) the percentage survival of mice as in FIG. 4, except that frozen pStemHeps were prepared accordingly to protocol C, as described in Table 2 of Example 2. Plot 1: APAP+pStemHeps (n=4), plot 2: APAP only (n=5); (Panels B-C) the number of proliferating cells in liver sections by immunohistochemistry using antibodies against K167 (MKI67) at 24 hours after injection of 1×10⁶ pStemHeps in animals that did received 700 mg/kg body weight of acetaminophen (APAP+ Cells; Panel B) or in untreated animals that did received APAP only (control APAP; Panel B). A K167 (MKI67) positive cell nuclei appears in dark dot. Original magnification ×10.

FIGS. 15A-B are plots showing the percentage survival of mice received 1500 mg/kg body weight of thioacetamide (TAA) and further received (plot 1, TAA+pStemHeps, n=9 for Panel A and n=5 for Panel B) or not (plot 2; TAA only, n=10) transplantation of 1×10⁶ frozen pStemHeps that were prepared accordingly to protocol C, as described in Table 2 of Example 2. Treated mice further received (Panel A) or not (Panel B) 1 mg/kg of tacrolimus (daily for 5 days and beginning 24 hours before cell injection).

FIG. 16 is a plot showing the levels of production of human AFP measured by ELISA in TAA-intoxicated C57BL/6 mice that did not received (CTRL), or that received 1×10⁶ frozen pStemHeps that were prepared accordingly to protocol C, as described in Table 2 of Example 2. Human AFP levels were determined in mice after 24 h post-transplantation. Each dot corresponds to an animal. * P<0.05.

FIGS. 17A-C are photographs showing the generation of spheroids prepared from freshly-prepared (Panel A) and from cryopreserved pStemHeps (Panel B) after plating and 2 days of culture into Aggrewell plates, as described in example 3. Panel C is a photograph showing viability of spheroids, as measured by Live/Dead assay immunofluorescent test. LIVE: esterase activity, DEAD: staining of Dead cells with ethidium homodimer-1, NUCLEI: cell nucleus staining with Hoechst 33342, LIGHT: phase contrast microscopy.

FIG. 18 is a histogram showing the relative levels of mRNAs of markers in freshly-prepared (pStemHep FRESH) or cryopreserved (pStemHep FROZEN) pStemHeps cultured as indicated in example 2 and in spheroids prepared from freshly-prepared (SPHE FRESH) or from cryopreserved pStemHeps (SPHE FROZEN) generated and cultured for 2 days as indicated in example 3, as determined by RT-qPCR, SOX17, HNF4A, and AFP. Relative gene expression levels are in Log 10 scale. All relative gene expression levels were significantly different from those of undifferentiated hESCs.

FIG. 19 is a photograph showing the expression of AFP, CK19, FOXA2, HNF4A and SOX17 and non-expression of ALB by 2-days cultured spheroids prepared from pStemHeps measured by immunofluorescent test.

FIGS. 20A-B are plots showing the expression of AFP and non-expression of ALB by spheroids (SPHE) that were prepared from pStemHeps and cultured for 2 days in Aggrewell 400™ by ELISA tests. pStemHeps and HLCs (generated as indicated in example 1) were used as positive controls for secretion of human AFP (Panel A) and human ALB (Panel B), respectively. * P<0.05.

FIG. 21 is a plot showing the percentage survival of mice (n=5) in each group) as in FIG. 4, except that frozen pStemHeps were prepared accordingly to protocol C, as described in Table 2 of Example 2, and in C57BL6 mice that did received 700 mg/kg body weight of acetaminophen and further transplanted with 2×10⁷ pStemHeps in the peritoneal cavity (plot1) or not (plot 2).

FIG. 22 is a plot showing the levels of in vivo production of human AFP measured by ELISA in 3 groups of C57BL/6 mice that did not received (CTRL, n=3), or that received cell-free/pre-molded alginate hydrogel transplanted in the peritoneal cavity (Alginate, n=5), or pre-molded alginate hydrogel containing 2 days-cultured spheroids that were prepared from 1×10⁷ pStemHeps and transplanted in the peritoneal cavity (SPHE Alginate, n=3). Human AFP levels were determined in mice after 24 h post-transplantation. Each dot corresponds to an animal. * P<0.05.

FIG. 23 is a photograph showing high viability of spheroids that were transplanted and harvested from animals depicted in FIG. 21 at day 8 post-transplantation, as measured by Live/Dead assay immunofluorescent test. After 8 days in the mice peritoneal cavity, most of cells in SPHE have high esterase activity (LIVE) and only few cells were positive to ethidium homodimer-1 (DEAD). Cell nucleus were stained with Hoechst 33342 (NUCLEI).

FIGS. 24A-B are histograms showing the relative levels of mRNAs of key markers of hepatic differentiation, i.e., AFP (Panel A) and HNF4A (Panel B), in spheroids that were prepared from pStemHeps cultured in vitro for 8 days (SPHE D8 in vitro) and in spheroids embedded in alginate hydrogel that were transplanted and harvested from animals depicted in FIG. 21 at day 8 post-transplantation (SPHE D8 Alginate), as determined by real-time RT-qPCR. The Relative gene expression levels are in Log 10 scale. * P<0.05.

FIG. 25 is a graph showing the size distribution of particles secreted by pStemHeps in cell supernatants using nanoparticle tracking analysis (NTA) with the PARTICLEMETRIX® ZetaView instrument.

FIG. 26A-C is a set of schemes and plots showing the detection of tetraspanin on extracellular vesicles (EVs) secreted by pStemHeps by ExoView device. The EVs are first captured on spots by anti-tetraspanin antibodies, then a combination of fluorescent-labelled antibodies against the same tetraspanins are applied and read (Panel A). The results demonstrate particles detection by fluorescence on the four different spots (particles are normalized by the read area, MIgG: isotype control) (Panel B). Finally, the distribution of the number of expressed tetraspanins in all spots are represented (Panel C). The 3 markers are CD63, CD81, and CD9.

FIG. 27 is a scheme showing the proteomics analysis on cell lysate, cell supernatant, and purified vesicles with a Venn diagram based on the detected proteins (threshold >1 PSM) from three samples.

FIG. 28 is a plot showing the expression of HGF in the supernatant of a culture of pStemHeps prepared accordingly to the protocol C as measured by ELISA test.

### EXAMPLES

The present disclosure and invention are further illustrated by the following examples. Unless stated otherwise, the term “pStemHeps” refers herein to the hepatic stem-like cells according to the invention.

### Example 1

### 1.1 Material and Methods

### a) Cell Culture

Human embryonic stem cell (hESCs) lines were derived under current Good Manufacturing Practice (cGMP) conditions on human fibroblast feeder layers and are available in research and clinical-grade formats. hESCs (ESI-BIO) were cultured in feeder-free conditions on culture dishes pre-coated with 5 μg/ml Laminin LN521 (BIOLAMINA®) in mTeSR1™ medium (STEM CELL TECHNOLOGIES®) at 37° C. in a 5% CO₂ incubator with daily media changes and were passaged using TrypLE™ (THERMOFISHER SCIENTIFIC®) and then cultured during 24 hours in the presence of 10 μM of the Rock inhibitor Y-27632 (STEM CELL TECHNOLOGIES®).

### b) Hepatic Differentiation In Vitro (Generation of the Population of Hepatic Stem Cells)

Cells (75,000 cells/cm²) were plated on laminin LN521 (BIOLAMINA®) at 5 μg/ml in mTeSR1™ (STEM CELL TECHNOLOGIES®) containing 10 μM of the Rock inhibitor Y-27632 (STEM CELL TECHNOLOGIES®). After 24 hours, to start differentiation (Day 0), hESCs maintenance medium was replaced by RPMI supplemented with B27 serum-free supplement (LIFE TECHNOLOGIES®) and cells were changed daily thereafter. During the first day of definitive endoderm differentiation induction, cells were cultured in the presence of 100 ng/ml Activin A (MILTENYI BIOTEC®), 50 ng/ml Wnt3a (R&D SYSTEMS®) and 3 μM CHIR-99021 (STEM CELL TECHNOLOGIES®). Then cells were cultured for 1 day in the presence of 100 ng/ml Activin A (MILTENYI BIOTEC®) and 50 ng/ml Wnt3a (R&D SYSTEMS®) and then for 3 days in the presence of 100 ng/ml Activin A (MILTENYI BIOTEC®). To induce the hepatic specification after endoderm formation, with 10 ng/ml fibroblast growth factor 10 (FGF-10) (MILTENYI BIOTEC®) and 10 ng/ml bone morphogenetic protein 4 (BMP-4) (R&D SYSTEMS®) were used for five days. After using this hepatic differentiation protocol A, pStemHeps were frozen in CryoStorm CS10 (STEM CELL TECHNOLOGIES®). In order to produce hepatocyte-like cells (HLCs) by hepatic maturation, cells were cultured in hepatocyte culture medium (HCM) (LONZA®) supplemented with 20 ng/ml hepatocyte growth factor (HGF) and 20 ng/ml oncostatin M (OSM) (MILTENYI BIOTEC®), the media was changed every 2 days.

### c) RNA Extraction and Real-Time Quantitative PCR

Total mRNA was extracted from culture using the RNeasy Mini kit (QIAGEN®) following the manufacturer's recommendations. Real-time reverse-transcription was performed starting from 5 ng RNA, with a one-step RT-PCR kit using Taqman® technology (AgPath-ID™ One-Step RT-PCR, LIFE TECHNOLOGIES®) and using the Applied Biosystems ViiA 7 Real-Time PCR System and the appropriate primers for Taqman assays (LIFE TECHNOLOGIES®): OCT4 (Hs00999632_g1), SOX17 (Hs00751752_s1), HNF4A (Hs00604435-ml), AFP (Hs00173490_ml), and GAPDH (Hs99999905_ml). The relative gene expression was calculated using the 2-ΔΔct quantification method after normalization to GAPDH values and expressed as fold of levels found in undifferentiated hESCs cells

### d) Flow Cytometry

Cells were harvested and incubated on ice with Fixable Viability Dye eFluor™ 450 (eBioscience™ 65-0863-14) for 20 min. The intracellular staining was carried out according to the manufacturer's instructions using Fixation/Permeabilization kit (eBioscience™ 00-5123-43 and 00-5223-56) in the presence or absence of primary antibodies against SOX17 (Allophycocyanin Goat anti-SOX17, R&D SYSTEMS®, #IC1924A) and HNF4A (Alexa Fluor 488 Mouse anti-HNF4A, SANTACRUZ®, #SC-374229). Detection of CXCR4 was performed without cell permeabilization and with anti-CXCR4 monoclonal mouse IgG2b antibody (R&D SYSTEMS®, #Mab173) and Alexa Fluor 568 anti-mouse IgG2b. The analysis was performed with a FACSCanto II (BD BIOSCIENCES®) and Flow Jo software (TREE STAR®, Ashland, OR, USA). Human primary hepatocytes were obtained from Biopredic International.

### e) Immunofluorescence Cell Staining Assay

Cultured cells were fixed with 4% paraformaldehyde for 15 min at room temperature, permeabilized with 0.5% Triton X-100 in PBS for 15 min and blocked with 1% BSA-0.1% Triton in PBS for 30 min. Primary antibodies were diluted in 11% BSA-0.1% Triton in PBS, and incubated 1 h at room temperature (mouse anti-AFP, SIGMA ALDRICH®, #A8452, 1/50; mouse anti-ALB, CEDARLANE®, #CL2513A, 1/300; rabbit anti-FOXA2, ABCAM®, #ab108422, 1/100; mouse anti-HNF4A, SANTACRUZ®, #SC-374229, 1/100; rabbit anti-OCT4, SANTACRUZ®, #SC-9081, 1/50). Secondary antibodies were diluted in 1% BSA-0.1% Triton in PBS and incubated for 1 hour at room temperature (Alexa Fluor 488 Donkey anti-mouse IgG, INVITROGEN®, #A21202, 1/200; Alexa Fluor 488 Goat anti-rabbit IgG, INVITROGEN®, #A21206, 1/200). Cells were mounted using coverslips and ProLong Gold Antifade Mountant (LIFE TECHNOLOGIES®). All pictures were observed under a Zeiss fluorescent microscope.

### f) Enzyme-Linked Immunosorbent Assay (ELISA) Analysis

Human AFP and ALB secreted into the culture medium were determined by the Human AFP Elisa Quantitation kit (ABCAM®) and the Human Albumin ELISA Quantitation kit (Bethyl; http://www.bethyl.com) following manufacturer's instructions.

Human AFP secreted into the sera of transplanted animals were specifically determined by the Human AFP Elisa Quantification Kit (EHAFP, THERMOFISHER SCIENTIFIC®) following the manufacturer's instructions.

### g) Animals and Induction of Acute Liver Failure (ALF)

Male NOD/SCID mice (6 weeks) were treated with 400 mg acetaminophen (APAP)/kg to induce acute liver failure (ALF) 3 hours prior to cell transplantation. ALF was evaluated by means of histological staining and determination of transaminases in the sera of treated animals. Three hours after the injection of APAP, animals received an intrasplenic injection of 1×10⁶ frozen pStemHeps in 50 μL RPMI/B27 medium (LIFE TECHNOLOGIES®). All assays were carried out using pStemHeps that had been cryopreserved and thawed. The control mice had received APAP intoxication and no treatment. At 24 h, mice were sacrificed under anesthesia (isoflurane). Blood, serum and liver were collected and stored at −80° C. until analysis. For histological analyses, formalin-fixed livers were dehydrated, embedded in paraffin blocks and cut into 5 μm sections. The liver sections were stained with haematoxylin and eosin (H&E), scanned with a digital slide scanner (Nanozoomer S360, Hamamatsu Photonics) at ×20 magnification. Digital images were converted to 8-bit grey scale and grey values were measured using ImageJ software to quantitatively evaluate the normal and the necrotic tissue areas. The livers of mice that were not intoxicated with APAP were used to define the grey values of a normal liver.

### h) PCR Alu

Genomic DNA was extracted from tissues using the Genomic DNA from organs and cells Kit (MACHEREY-NAGEL®) following the manufacturer's recommendations. Alu PCR is conducted using two primers: hAluR: 5′-TTT TTT GAG ACG GAG TCT CGC TC-3′ (SEQ ID NO: 1) and hAluF: 5′-GGC GCG GTG GCT CAC G-3′ (SEQ ID NO: 2). PCR is carried with Herculase Kit (AGILENT®) out in a total volume of 25 μL with 10 ng of genomic DNA. PCR is carried with Herculase® Kit (AGILENT®) out in a total volume of 25 μL with 10 ng of genomic DNA. PCR running conditions are the ones recommended by the manufacturer.

### i) Statistical Analysis

Data are expressed as mean values ±SEM. Statistical analysis was made using the GraphPad Prism® 7 software (GraphPad Software, San Diego, CA). Statistical significance was assessed using the Student's t test for comparisons between groups. Survival data were analyzed with the Kaplan-Meier test. The statistical significance of the survival rates was determined by a log-rank test. For all tests, P<0.05 was considered significant (*).

### 1.2 Results

a) The Differentiation of hESCs cGMP into Hepatic Stem Cells (pStemHeps)

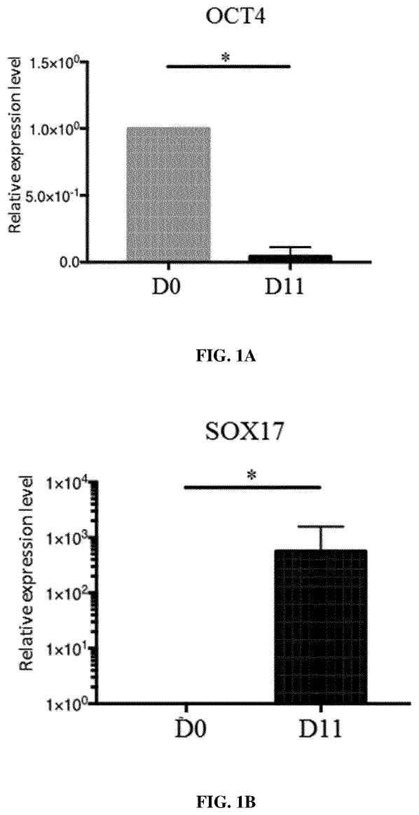

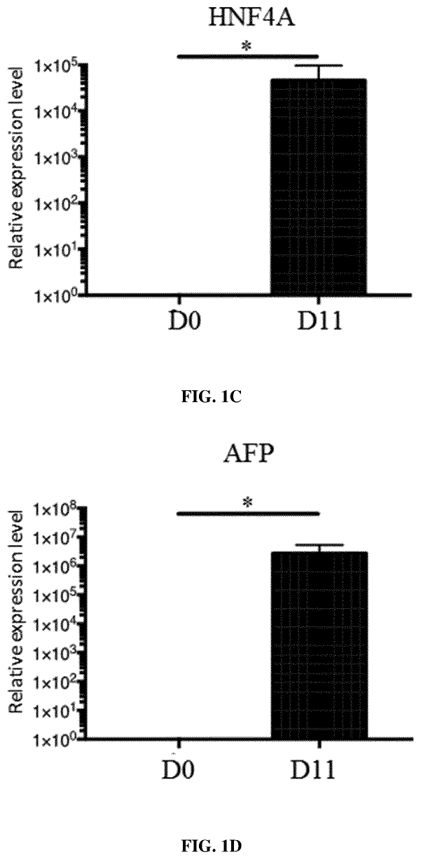

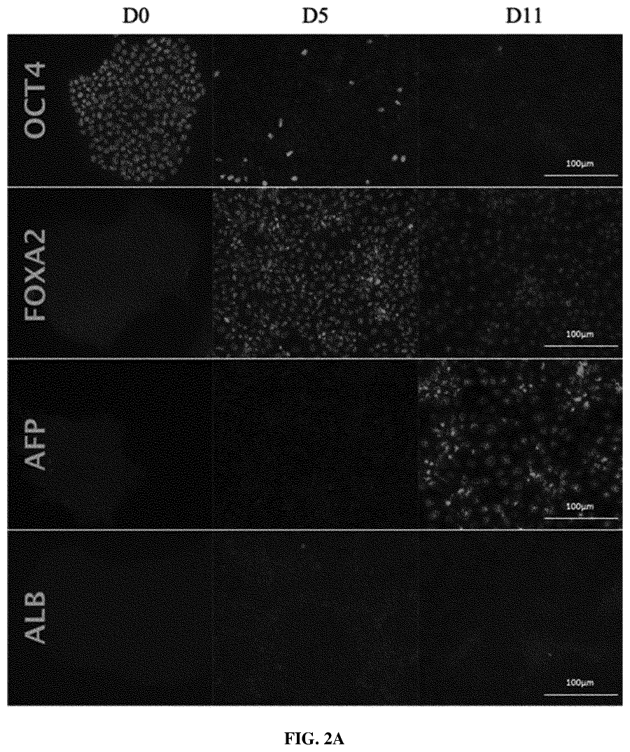

At day 0 of the differentiation protocol, the hESCs cGMP culture were positive for the pluripotency markers octamer-binding transcription factors 4 (OCT4) (FIGS. 1 and 2A). The hESCs cGMP were subjected to a three steps differentiation protocol. The cells were induced into definitive endoderm (DE), followed by 5 days of hepatic specification where the cells differentiated into hepatic stem-like cells (pStemHeps). FIG. 2A shows the kinetics of expression of keys markers of pluripotency (OCT4), DE (FOXA 2), hepatic progenitors (AFP) and mature hepatocyte (ALB): disappearance of OCT4 expression, expression of FOXA2 after DE induction (Day 5), expression of AFP after hepatic induction (Day 11) and no expression of albumin (ALB). At day 11, pStemHeps expressed AFP, CXCR4 (DE marker), FOXA2, HNF4A and SOX17 (DE marker) as measured by RNA, immunofluorescence and/or cytofluorimetry analyses (FIGS. 1B-D and 2A-C). Accordingly, pStemHeps did secrete AFP (FIG. 2D) but not ALB (FIG. 3C).

Cryopreservation and thawing procedures have been reported to have detrimental effects on the viability and function of primary human hepatocytes when compared to freshly isolated cells. The successful cryopreservation of pStemHeps successfully retained high viability on a period of two to nine months (Table 1).

TABLE 1
Cryopreserved and thawing pStemHeps

| | Cell | Cell viability | Cell | | |
|---|---|---|---|---|---|
| | viability | average | viability | | |
| | before | before | after | Cell viability | Freezing |
| # | freezing | freezing | freezing | average after | time |
| Prod | (%) | (%) | (%) | freezing (%) | (month) |
| 1 | 94 | 93 | 92 | 91 | 3 |
| 2 | 91 | | 90 | | 2 |
| 3 | 97 | | 96 | | 9 |
| 4 | 90 | | 85 | | 4 |

b) The Potency of hESCs to Differentiate into HLCs

After hepatic maturation of pStemHeps into HLCs, the morphology of the differentiated cells shared many characteristics with adult primary hepatocytes, including a polygonal shape, distinct round nuclei or double nuclei, and numerous vacuoles or vesicles (FIG. 3B). HLCs secreted ALB, an important function displayed by mature hepatocytes (FIG. 3C). Cell immunostaining showed that HLCs were positive for ALB, AFP and HNF4A (FIG. 3A).

c) pStemHeps Rescue from APAP-Induced ALF

To assess the therapeutic potential of pStemHeps in vivo, the model of acetaminophen toxicity (APAP) in immuno-compromised mice was used, which mimics ALF. This model was first chosen because consistent hepatotoxicity has been shown in murine models and hepatocyte damage in human liver. In western countries, the paracetamol intoxication is the first cause of ALF.

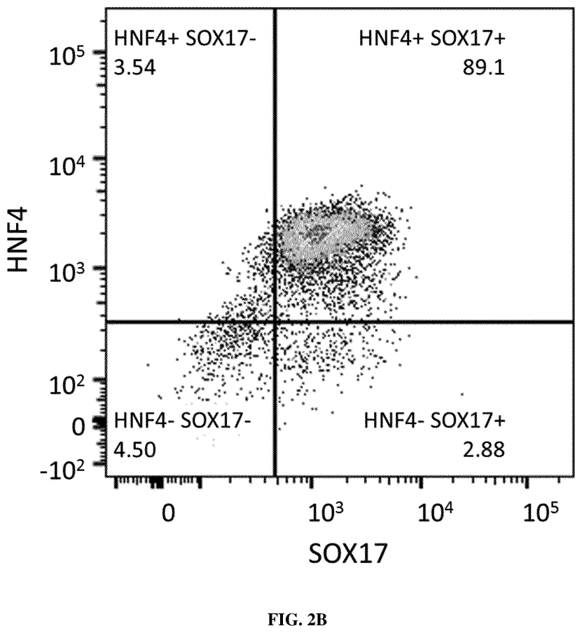

An acetaminophen dose of 400 mg/kg body weight resulted in a rapid death of control animals as soon as 24 hours after the administration of APAP. pStemHeps transplantation significantly increase animal survival (>90% survival), when compared to control ALF animals (FIG. 4).

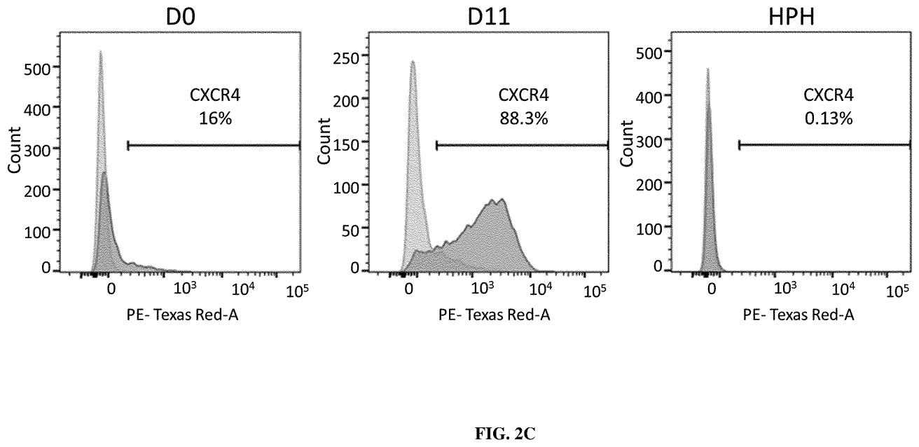

Accordingly, induction of ALF was concomitant with the rapid release of alanine aminotransferase (ALAT, a major liver injury marker) in the blood. pStemHeps transplantation resulted in reduction of ALAT values, as soon as 24 hours after cell therapy, when compared to control ALF animals (FIG. 5).

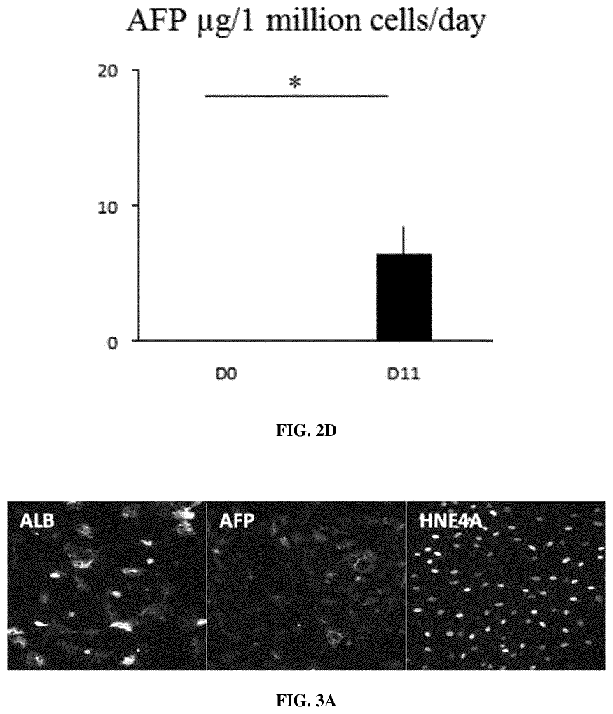

Furthermore, histological analysis indicated a higher extent of liver necrosis in control animal group receiving only APAP compared to animal group receiving APAP and pStemHeps within 24 hours post-cell transplantation (FIG. 6), showing a rapid therapeutic benefit of pStemHeps therapy.

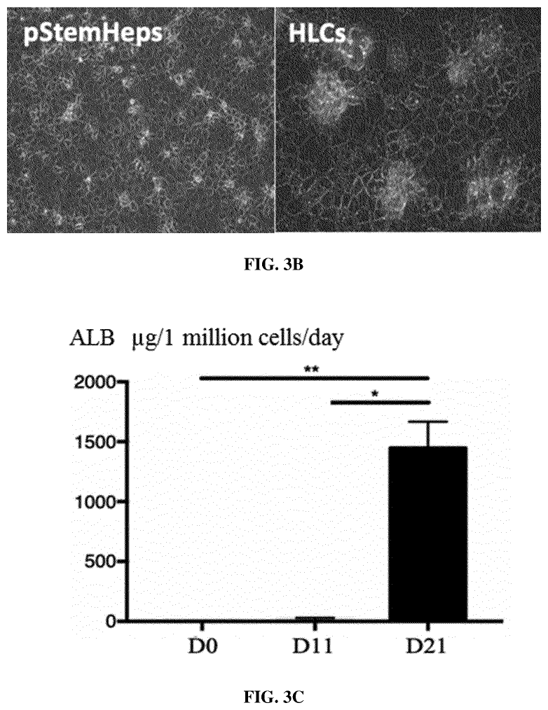

Human AFP was detected in the sera of transplanted mice and not in the sera of control non-transplanted mice at 24 h post cell injection (FIG. 7), showing fast cell recovery and functionality after pStemHep thawing.

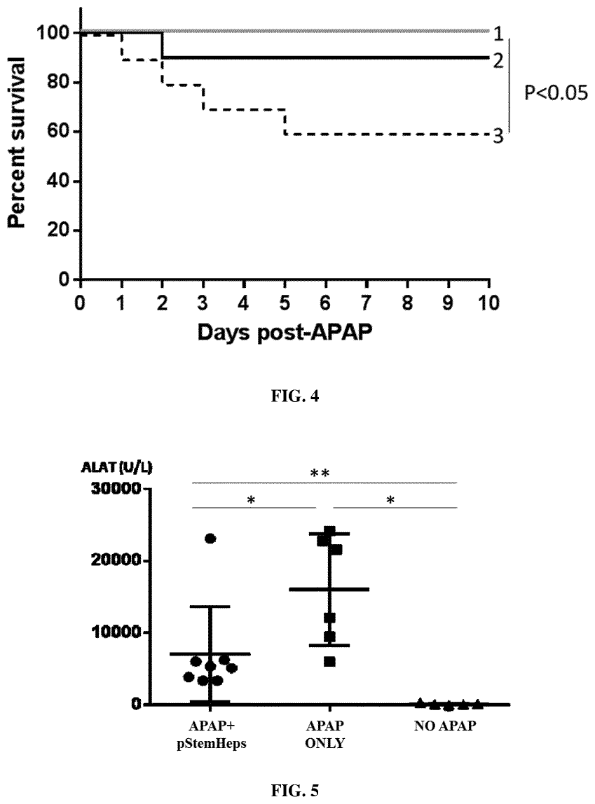

In order to investigate whether the transplanted cells were engrafted within the livers of the recipient mice, the presence of human pStemHeps was detected using human Alu PCR (specific sequence that is highly repeated). Human cells were detected in liver after only 24 h post transplantation (FIG. 8).

### 1.3 Conclusion

Cryopreservation and thawing procedures have been reported to have detrimental effects on the viability and function of primary human hepatocytes when compared to freshly isolated cells (Terry et al., 2010). The successful cryopreservation of human hepatic progenitors that retain high viability, as well as the ability to be cultured and further differentiated, would allow for long-term banking of the cells required for subsequent research and clinical applications. The hepatic stem-like cells were able to proliferate and express hepatic specific markers such as HNF4A and AFP. When further maturated, cells showed liver specific functions such as albumin secretion.

After cell infusion, pStemHeps recover rapidly from cryopreserved state, engrafted and were able to protect mice from lethal acute liver failure. Cells were detected in the liver of the transplanted mice after 24 h. A significant and rapid decrease in serum ALAT and liver tissue necrosis was also reported as compared to controls APAP-ALF mice.

Here, it is shown that frozen immature hepatocytes produced from human pluripotent stem cells are able to rescue mice from APAP-ALF by accelerating liver regeneration of healthy tissue. pStemHeps rapidly recover and become functional within 24 h post-transplantation. Altogether, these results show that higher regeneration of the healthy liver tissue upon pStemHeps transplantation may thus benefit treatment of fulminant liver failure, such as ALF, and also benefit treatment of fulminant liver failure with preexisting chronic liver diseases, such as ACLF. Indeed, similarly to ALF, ACLF benefits from a regeneration of healthy liver tissue within the diseased liver to treat fulminant liver failure.

### Example 2

### 2.1 Material and Methods

### a) Cell Cultures

Human embryonic stem cell (hESCs) lines were derived under current Good Manufacturing Practice (cGMP) conditions on human fibroblast feeder layers and are available in research and clinical-grade formats. hESCs (ESI-BIO) were cultured in feeder-free conditions on culture dishes pre-coated with 5 μg/mL Laminin LN521 (BIOLAMINA®) or with 0.5 μg/cm² vitronectin (GIBCO™) in mTeSR1 medium (STEM CELL TECHNOLOGIES®) at 37° C. in a 5% CO₂ incubator with daily media changes, and were passaged using TrypLE™ (THERMOFISHER SCIENTIFIC®) and then cultured during 24 hours in the presence of 10 μM of the Rock inhibitor Y-27632 (STEM CELL TECHNOLOGIES®).

### b) Hepatic Differentiation In Vitro and Characterization of the Hepatic Stem Cells

Cells (20,000 to 50,000 cells/cm²) were plated on laminin LN521 (BIOLAMINA®) at 5 μg/ml (protocol A and B) or LN511 (iMatrix 511, AMSBIO®) at 0.0625 μg/cm² (protocol C) cultured in mTeSR1™ (STEM CELL TECHNOLOGIES®) with daily culture changes, and were passaged using TrypLE™ (THERMOFISHER SCIENTIFIC®) and then cultured during 24 hours in the presence of 10 μM of the Rock inhibitor Y-27632 (STEM CELL TECHNOLOGIES®).

After 48 hours, hepatic differentiation of hESCs was then started as performed in example 1 to generate pStemHeps, according to the Table 2, which provides the cultures' protocols.

TABLE 2
protocols for the preparation of hepatic stem cells from hESCs

| Protocol | | |
|---|---|---|
| number | Endoderm induction* | Hepatic differentiation* |
| A | d0-d1: ACTA + Wnt3A + CHIR-99021 | d5-d11: BMP4 + |
| | d1-d2: ACTA + Wnt3A | FGF10 |
| | d2-d5: ACTA | (matrix: LN521) |
| | (matrix: LN521) | |
| B | d0-d2: CHIR99021 | d3-d8: BMP4 + FGF10 |
| | (matrix: LN521) | d8-d10: HGF + |
| | | CHIR99021 |
| | | (matrix: LN521) |
| C | d0-d2: CHIR99021 | d3-d8: BMP4 + FGF10 |
| | (matrix: iMatrix 511) | d8-d10: HGF + |
| | | CHIR99021 |
| | | (matrix: iMatrix 511) |

*Duration of the step with ACT-A (100 ng/ml), Wnt3A (50 ng/ml), CHIR99021 (3 μM), BMP4 (10 ng/ml), FGF10 (10 ng/ml), HGF (20 ng/ml).

The population of hepatic stem-like cells generated by the protocols above were characterized in vitro and in vivo.

For in vitro characterization, parameters such as the harvested density of cells, the yield, the viability of the cells, the levels of markers for DE specification such as FOXA2 and SOX17, for hepatic stem cell specification such as AFP, APOA1, APOB, HNF1B HNF4A, TBX3, KRT19 and TTR, and for mature hepatocytes specification such as ALB, ASGR1, CYPs F9, NAGS, and UGT1A1 were assessed.

The level of markers was assessed by real-time RT-PCR, ELISA, FACS, immunofluorescence cell staining as mentioned. Information relative to the qPCR primers for Taqman assays are indicated in example 1 and with qPCR primers (LIFE TECHNOLOGIES®) for FOXA2 (Hs00232764_ml), HNF1B primers (Hs01001602-ml), TBX3 (Hs00195612_ml), TTR (Hs00174914-ml). Information relative to the antibodies for immunofluorescence cell staining are indicated in example 1 and with anti-EPCAM (R&D SYSTEMS®, #AF960), anti-CK19/KRT19 (DAKO®, #M0888), anti-SOX17 (R&D SYSTEMS®, #AF1924) anti-KI67 (ABCAM®, #Ab15580), and Alexa Fluor 568 Donkey anti-goat IgG (INVITROGEN®, #A11057).

Alternatively, the levels of markers were assessed by mRNA expression profiling by 3′ DGE (DGE-Seq). RNA-sequencing protocol was performed on 10 ng of total RNA to determine the number of mRNA molecules per million of total mRNA molecules as described by Kilens et al. (2018).

### c) Animals and Induction of Acute Liver Failure (ALF)

For in vivo characterization, the hepatic stem cells were assessed for their ability to rescue APAP-induced ALF in NOD/SCID mice at a dose of 400 mg/kg or 720 mg/kg (see Example 1 above).

Alternatively, the NOD/SCID mice with APAP-induced ALF have undergone surgery as to remove approximately ⅓ of the liver prior to the transplantation with 1×10⁶ cryopreserved pStemHeps, as indicated.

Alternatively, male C57BL/6 mice (6 weeks) were treated with 1,500 mg thioacetamide (TAA)/kg to induce ALF 24 hours prior to transplantation of 1×10⁶ cryopreserved pStemHeps that was performed as described in example 1.

At time of sacrifice, blood was collected and serum aliquots were protected from light and stored at −80° C. until analyses measuring human AFP by ELISA as described in example 1.

The presence of K167 (MKI67)-positive cells was assessed by immunohistochemistry on formalin-fixed/paraffin-embedded liver sections (3 μm) at 24 hours after injection of 1×10⁶ pStemHep (prepared as in protocol C; see Table 2) in animals that did received 700 mg/kg body weight of APAP or in untreated control animals that did received APAP only. After paraffin was extracted from sections, endogenous avidin/biotin binding sites were blocked using an Avidin/biotin blocking kit (THERMOFISHER SCIENTIFIC®, #00-4303) and endogenous peroxidase activity was inhibited by incubation for 10 minutes in a 3% H₂O₂ solution in PBS. After incubation in Animal Free Blocker (VECTOR LABORATORIES®, #SP-5030-250) for 1 hour, rabbit polyclonal anti-KI67 primary antibodies (ABCAM®, #ab15580) diluted 1:1,000 in Animal Free Blocker was applied overnight at 4° C. The K167-positive cells were revealed with biotinylated goat anti-rabbit immunoglobulin and streptavidin-peroxidase (ABCAM®; #ab64261) using diaminobenzidine as a chromogenic substrate. Slides were counter-stained with hematoxylin and xylene.

### d) Statistical Analysis

Statistical analyses were performed as indicated in example 1.

### 2.2 Results

a) The Differentiation of hESCs cGMP into pStemHeps According to 3 Different Protocols

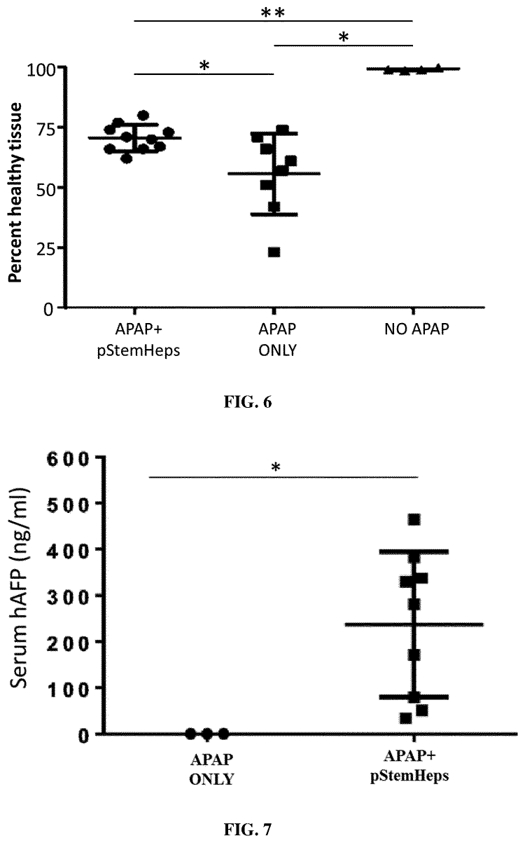

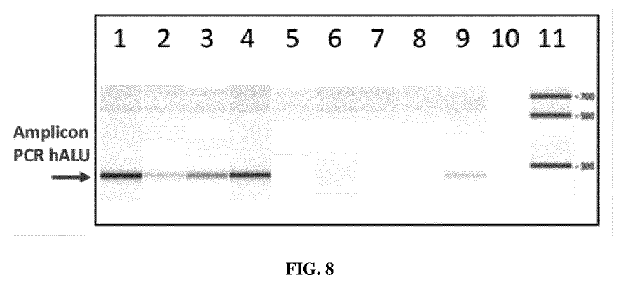

hESCs have been differentiated in hepatic stem cells accordingly to one among 3 protocols (different matrix and cocktails for hepatic differentiation) as in Table 2. pStemHeps that were produced according to the different protocols all expressed AFP but also FOXA2, HNF1b, HNF4A, TBX3 and TTR as assessed by RTqPCR analyses (FIG. 9). Furthermore, RNAseq analyses of pStemHeps (protocol A and B) showed high expression of the following genes: AFP, APOA1, APOA2, APOB, APOE, CD164, CD24, DPP4, EPCAM, G1A1, GSTA2, KRT18, KRT19, SEPP1, SOD1, SPARC, TTR, VIM, VTN (FIG. 10). They also expressed these genes at a lower relative level: APOA4, BMP2, BMP4, DLKT, GATA4, GATA6, GSTA1, HNF1B, HNF4A, SMAD7, TBX3. And they did not express the following genes characteristic of mature hepatocytes: ABCB11, ASGR1, CYP1A2, CYP2A6, CYP2B6, CYP2B7P, CYP2C9, CYP2E1, CYP3A4, CYP3A7, F9, NAGS, UGT1A1 and did not express PDX1.

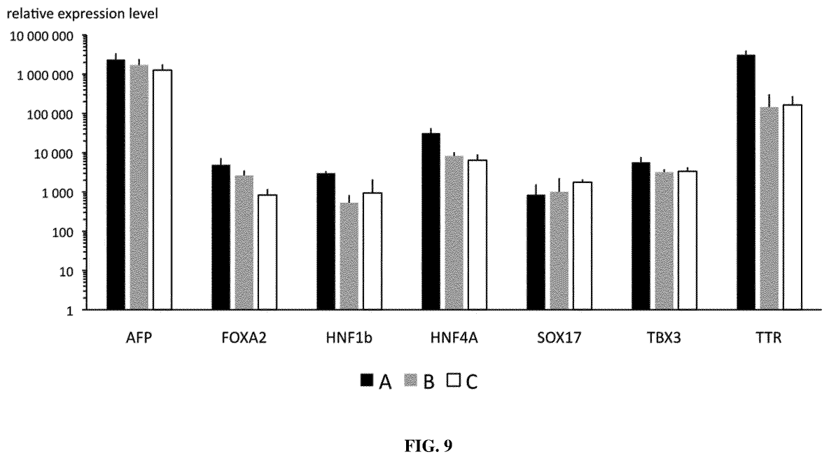

Accordingly, pStemHeps that were produced using Protocol A, B and C did produce and secrete AFP proteins and not ALB proteins (FIGS. 11A and 11B). FIG. 11B also shows that pStemHeps were positive for HNF4A, SOX17, EPCAM, CK19 (KRT19), FOXA2 proteins. pStemHeps were also positive for KI67 proteins, a marker of proliferating cells (FIG. 11B).

b) pStemHeps Rescue from APAP-Induced ALF

Cryopreserved pStemHeps generated by protocols A, B and C were assessed for their ability to rescue APAP-induced ALF in NOD/SCID mice.

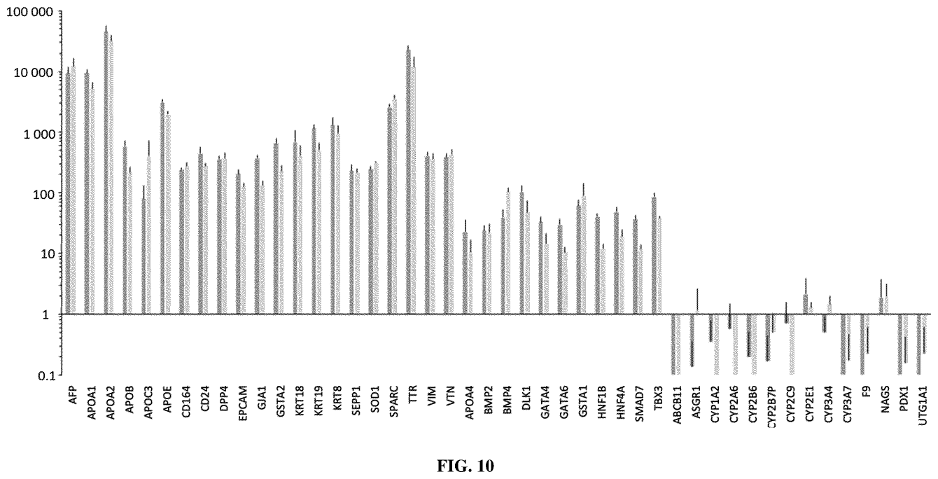

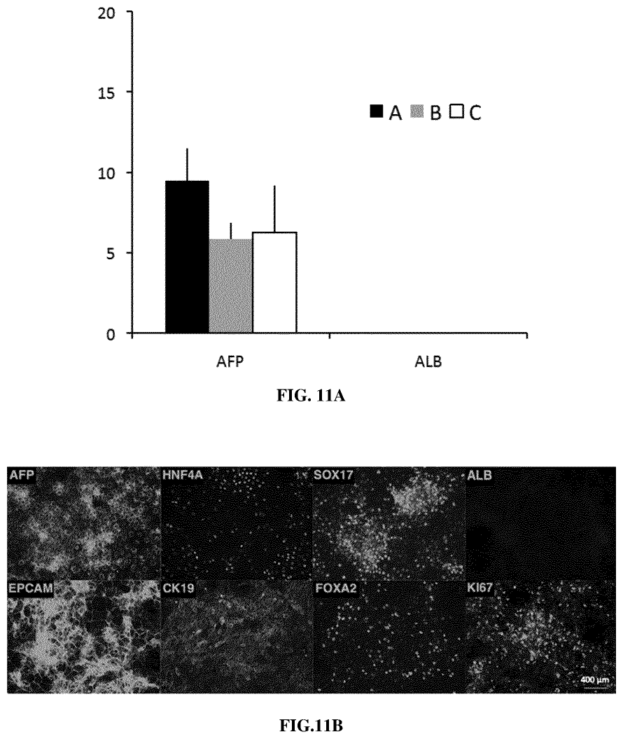

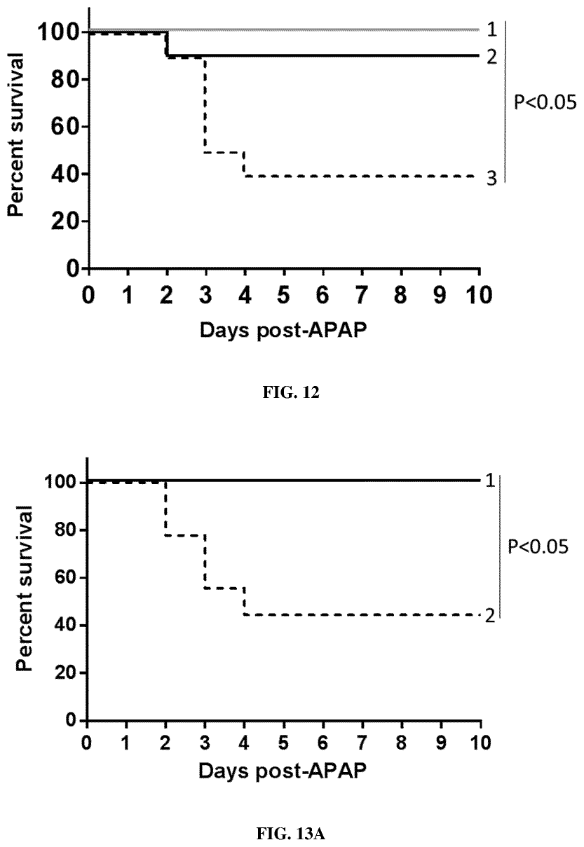

FIGS. 4, 12, 13A-B and 14A respectively show that the pStemHeps generated by these protocols all promote a significant increase of survival of NOD/SCID mice with acetaminophen-induced ALF (FIG. 4: protocol A, FIG. 12: protocol B, FIGS. 13A-B: protocols B and C, respectively, FIG. 14A: protocol C).

pStemHeps generated with protocols B and C were able to rescue acetaminophen-induced ALF in NOD/SCID mice having undergone surgery to remove ⅓ of the liver before transplantation with these cells (see FIG. 13A-B, respectively).

pStemHeps are able to rescue ALF in NOD/SCID mice intoxicated at a high dose of acetaminophen resulting in 100% mice death within 2 days in untreated control mice group (FIG. 14A).

As shown in FIG. 14B-C, pStemHeps promote a significant increase of proliferating cells in the liver of NOD/SCID mice with acetaminophen-induced ALF (FIG. 14B) at 24 h post-cell injection as compared to untreated control mice with acetaminophen-induced ALF that did not received pStemHeps (FIG. 14C). These results demonstrate faster liver regeneration after pStemHeps cell therapy in APAP-induced ALF mice.

c) pStemHeps Rescue from Non APAP-Induced ALF

Cryopreserved pStemHeps generated were assessed for their ability to rescue ALF induced in mice using hepatotoxin thioacetamide (TAA) as an animal model of non-acetaminophen induced human ALF.

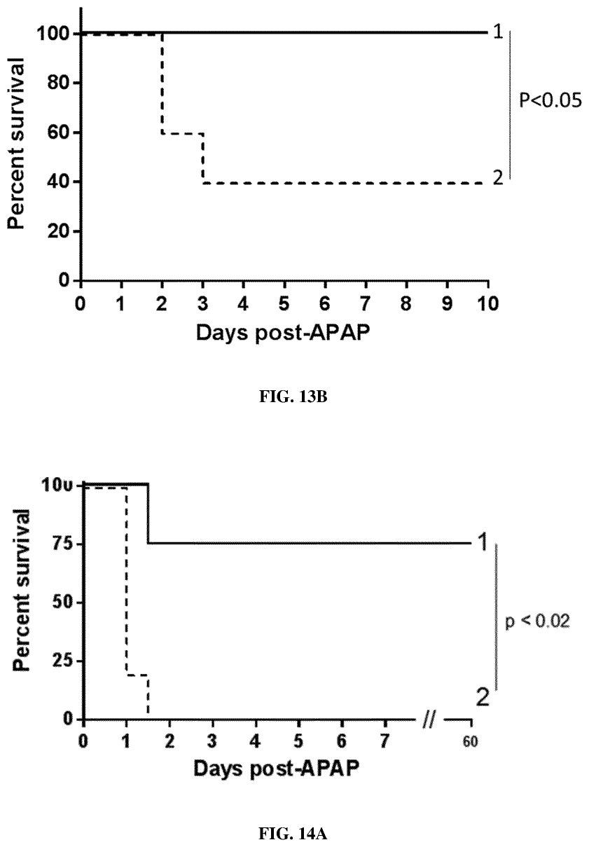

FIG. 15A-B shows that the pStemHeps generated by protocol rescue immunocompetent mice from TAA-induced ALF, in the presence or in the absence of tacrolimus (immunosuppressive agent). Altogether, these results provide evidence about the low immunogenicity of the cells.

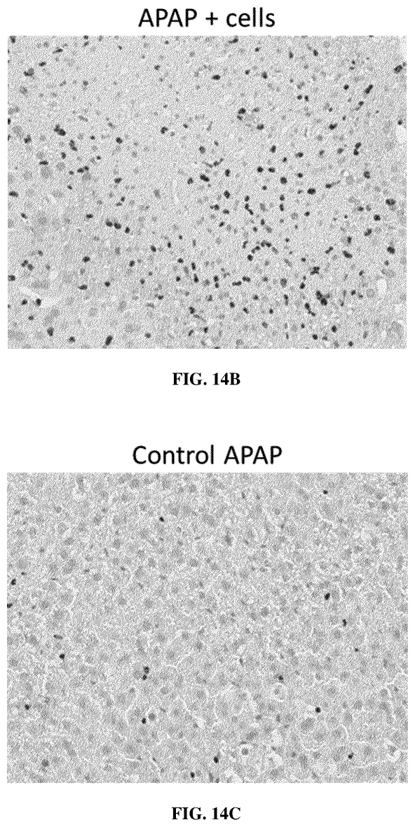

FIG. 16 shows that human AFP was detected in the sera of transplanted mice and not in the sera of control non-transplanted mice at 24 h post cell injection, showing fast cell recovery and functionality after pStemHeps thawing in a non-APAP-induced ALF in mice.

### 2.3 Conclusions

Here is shown that the obtention of hepatic stem-like cells (pStemHeps) preparation from hESCs are not limitative steps, since several protocols may be implemented with significantly equivalent therapeutic results to treat acute liver failure in a mouse model. Furthermore, pStemHeps can rescue APAP-induced and non-APAP induced ALF. The pStemHeps became therapeutically active fast enough (within 24 hours) after cell thawing and cell transplantation to rescue mice from ALF (first death occurring within 24 hours) and/or in absence of immunosuppression. Altogether, these results suggest that pStemHeps transplantation allow higher liver regeneration of healthy tissue in conditions of fulminant liver failure, such as ALF, as well as in conditions of fulminant liver failure with preexisting of chronic liver diseases, such as in ACLF. Again, as for ALF, ACLF benefits from a regeneration of healthy liver tissue within the diseased liver to treat fulminant liver failure.

### Example 3

### 3.1 Materials and Methods

a) Generating Spheroids from pStemHeps

At the end of the pStemHeps specification differentiation stage (see protocols described in example 2), pStemHeps cells were rinsed with Ca/Mg free PBS (GIBCO®) and incubated with TrypLE™ (LIFE TECHNOLOGIES®) for 10 minutes at 37° C. RPMI/B27 (LIFE TECHNOLOGIES®) was added to the dissociated cell suspension and cells were gently flushed to be fully dissociated. Cells were then plated into Aggrewell 400™ plates (STEM CELL TECHNOLOGIES®) to attain a final density of 1,000 cells/spheroid in complete HCM (LONZA®) supplemented with 10 μM of Y27632 (STEM CELL TECHNOLOGIES®), 20 ng/ml HGF (MILTENYI®), 20 ng/ml OSM (MILTENYI®) and 1 μM dexamethasone (SIGMA ALDRICH®) (D0 SPHE). Cells were incubated for 48 hours at 37° C., 5% CO₂ with no medium change.

### b) Gene Expression Analysis

The level of markers was assessed by RT-qPCR and ELISA, as mentioned above.

For immunofluorescence cell staining, spheroids were rinsed with Ca/Mg supplemented PBS and fixed for 30 minutes using 4% PFA, permeabilized with 0.5% Triton in PBS for 15 minutes. Cell Immunostaining were performed by incubating spheroids in PBS containing 0.1% Triton and 1% BSA (blocking buffer) for 1 h with the primary antibodies, and 1 h with the appropriate secondary antibodies at room temperature (Table 3).

TABLE 3
Antibodies used for immunofluorescent staining of spheroids

| | | |
|---|---|---|
| Target Species | Provider's Reference | Dilution |

Primary antibodies

| | | |
|---|---|---|
| ALB ms | CEDARLANER #CL2513A | 1/300 |
| CK19 ms | DAKOR #M0888 | 1/100 |
| AFP ms | SIGMA ALDRICH ® #A8452 | 1/50 |
| HNF4A ms | SANTACRUZ ® #SC-374229 | 1/100 |
| FOXA2 rbt | ABCAM ® # AB108422 | 1/100 |
| SOX17 gt | R&D SYSTEMS ® #AF1924 | 1/40 |

Secondary antibodies

| | | |
|---|---|---|
| Dk anti-ms AF488 | INVITROGEN ® #A21202 | 1/200 |
| Gt anti-rbt AF568 | INVITROGEN ® #A11036 | 1/200 |
| Dk anti-gt AF568 | INVITROGEN ® #A11057 | 1/200 |

### b) Cell Viability

To assess in vitro cell viability, 0.4 μM calcein-AM and 4 μM ethidium homodimer-1 (LIVE/DEAD viability/cytotoxicity kit, LIFE TECHNOLOGIES®) and 10 μg/ml Hoechst 33342 (LIFE TECHNOLOGIES®) were added to the culture medium for 30 minutes before imaging.

### c) Transplantation of Spheroids and In Vivo Functionality

Before transplantation, spheroids were embedded in alginate hydrogels. For this, two-days cultured spheroids were generated as described above, harvested from Aggrewell, centrifuged at 100×g for 5 min and resuspended in calcium/magnesium free PBS (LIFE TECHNOLOGIES®). Spheroids were mixed with 8.9% ultra-pure sodium alginate, with low viscosity and high glucoronic acid (Pronova SLG20, NOVAMATRIX®) and then gently mixed with 0.0225 M CaCO₃ (SIGMA ALDRICH®), and 0.045 M glucono-d-lactone (SIGMA ALDRICH®) to attain a final cell concentration of 20×10⁶/ml (approximately 2×10⁴ spheroids). Gelation of 250 μl hydrogels took place at room temperature for 3 min. Two 250 μl pre-molded alginate hydrogel containing or not spheroids were intraperitoneally transplanted under laparotomy into immunocompetent C57BL/6.

To assess in vivo viability of spheroids, alginate hydrogels were harvested from transplanted animals at day 8 post-transplantation, and dissociated using a solution of PBS without calcium and magnesium (LIFE TECHNOLOGIES®) containing 0.1 M EDTA (LIFE TECHNOLOGIES®), and 0.2 M sodium citrate tribasic (SIGMA ALDRICH®). After complete hydrogel dissociation, spheroids were spun down at 100×g for 5 min, incubated in fresh RPMI/B27 containing 0.4 μM of calcein-AM and 4 μM ethidium homodimer-1 (LIVE/DEAD viability/cytotoxicity, LIFE TECHNOLOGIES®) and 10 μg/ml Hoechst 33342 (LIFE TECHNOLOGIES®) for 90 minutes before imaging.

Gene expression levels in spheroids was assessed at 8 days post-transplantation by real-time RT-PCR after harvesting and dissociating alginate hydrogels as described above. Serum AFP of transplanted animals were determined by the AFP Elisa Quantification Kit specific for human AFP (EHAFP, THERMOFISHER SCIENTIFIC®) following the manufacturer's instructions.

### d) Statistical Analysis

Statistical analyses were performed as indicated in example 1

### 3.2 Results

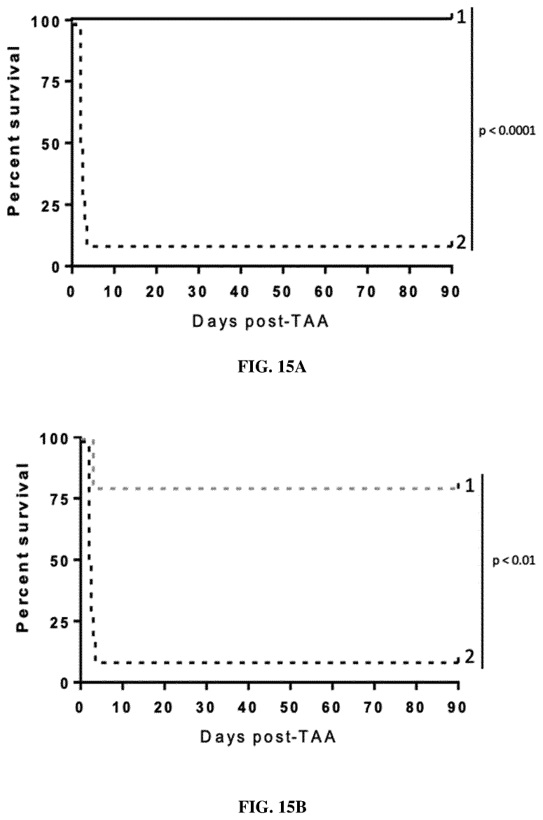

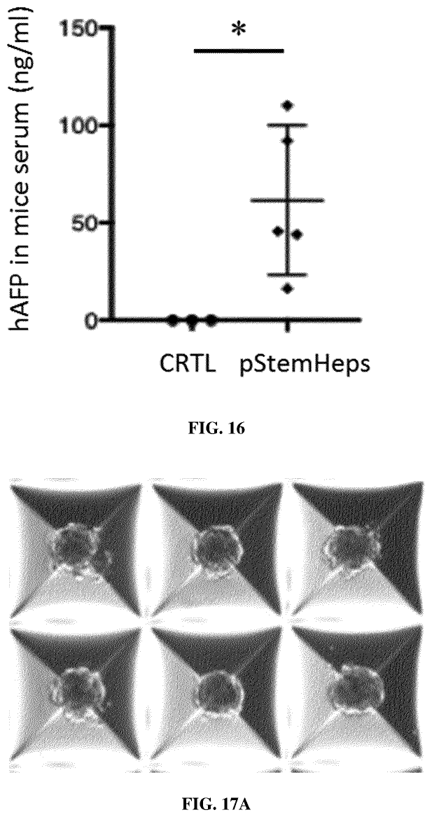

As shown in FIG. 17A-C either freshly-prepared or cryopreserved pStemHeps could aggregate and form spheroids in 48 hours after plating into Aggrewell plates. At harvest, spheroids had high esterase activity and were not positive for ethidium homodimer-1 staining. Only non-aggregated cells were positive to ethidium homodimer-1 and did not show any esterase activity. These results showed high viability of all spheroids. In addition, spheroids prepared from freshly-prepared or cryopreserved pStemHeps expressed high levels of AFP, HNF4A and SOX17 mRNA, similar to those of pStemHeps from which they were prepared (FIG. 18).

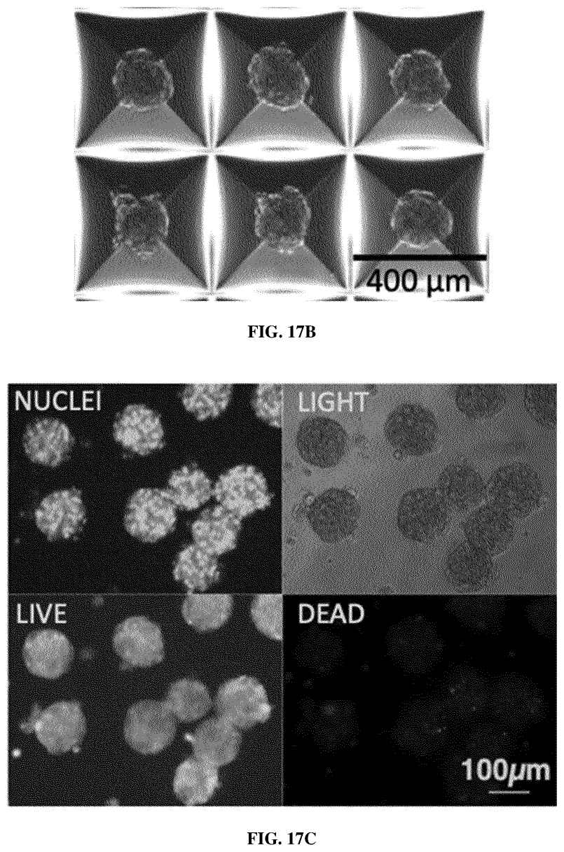

An immunofluorescent assay was further performed on spheroids, in order to assess the levels of expression of some key markers of hepatic differentiation. Results showed that spheroids express AFP, CK19 (KRT19), FOXA2, HNF4A, and SOX17 proteins, whereas they did not express the ALB proteins (FIG. 19).

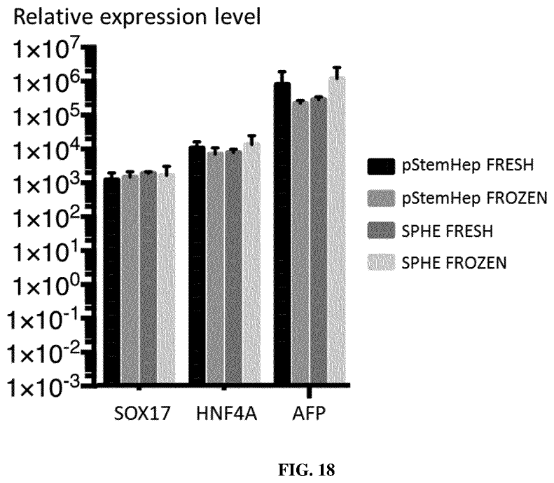

Finally, ELISA for AFP performed on cell supernatant samples confirmed that spheroids secreted AFP, which level was similar level to that of pStemHeps (FIG. 20A). In addition, no secretion of ALB by the 2 days-cultured spheroids was found, in contrast to HLCs (FIG. 20B).

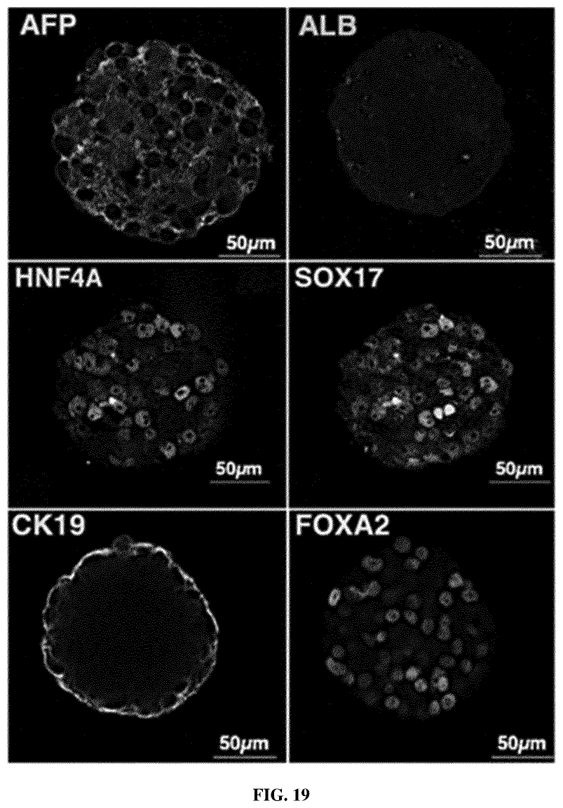

To evaluate the in vivo functionality of spheroids, two pre-molded alginate hydrogels containing a total of about 10×10⁶ pStemHeps were intraperitoneally transplanted in CB57BL/6 mice. In addition, 2×10⁷ pStemHeps were intraperitoneally transplanted in CB57BL/6 ALF-mice and rescue the survival (FIG. 21).

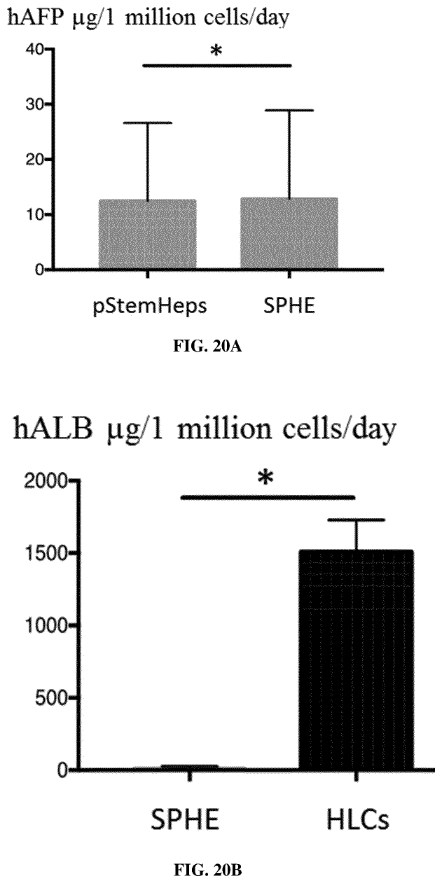

FIG. 22 shows that human AFP was detected in the sera of C57BL/6 mice at 2 days post-transplantation with pre-molded alginate hydrogels containing spheroids at a mean level of about 320 ng/ml.

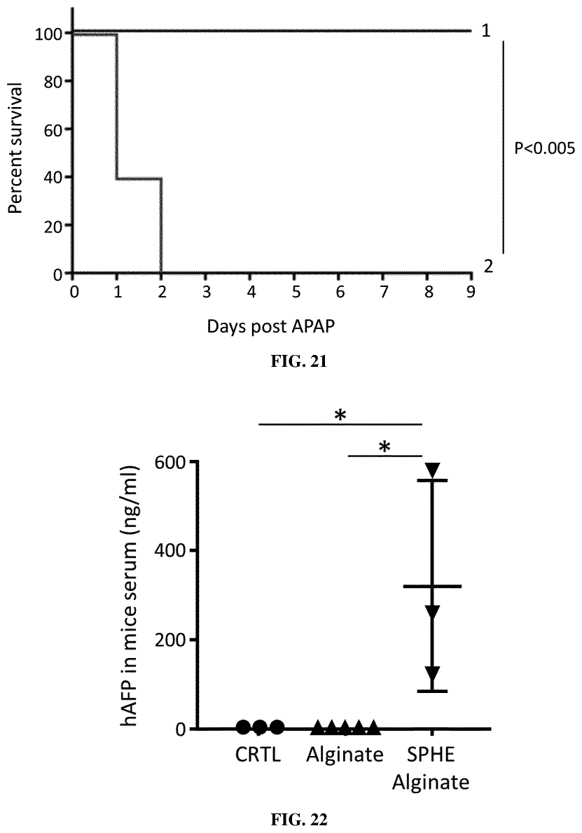

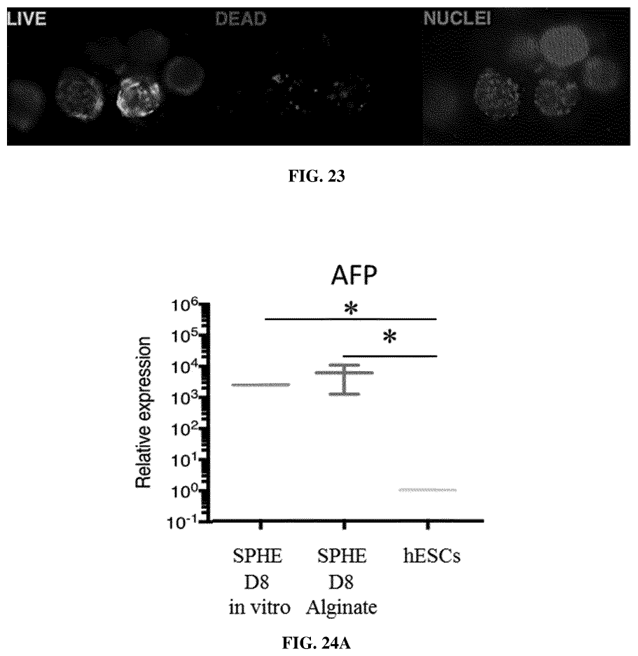

After 8 days post-transplantation, spheroids embedded in alginate hydrogels are highly viable (FIG. 23) and expressed AFP and HNF4A at a similar level than that of spheroids (non-embedded in alginate hydrogel) cultured in vitro during 8 days (FIG. 24A-B).

### 3.3 Conclusions

Here is shown that it is possible to quickly and efficiently generate spheroids from either freshly-prepared or cryopreserved isolated pStemHeps cells. Spheroids show high viability in vitro and maintain their progenitor status as similar to pStemHeps cells, in particular with expression of the AFP marker and no expression of the ALB marker. These spheroids, which were embedded in alginate hydrogels, have the ability to secrete AFP in vivo and survive for at least 8 days in the peritoneal cavity of immune competent animals, which is a time sufficient enough to rescue mice from acute liver failure (death occurs within 5 days, see example 1 and example 2). In addition, pStemHeps transplanted in the in peritoneal cavity rescue mice from ALF. Altogether, these results support that pStemHeps embedded in hydrogels may thus benefit treatment of fulminant liver failure, such as ALF, and also benefit treatment of fulminant liver failure with preexisting chronic liver diseases, such as ACLF.

### Example 4

### 4.1 Materials and Methods

a) Purification and Size Distribution of Extracellular Vesicles (EVs) Secreted by pStemHep in Cell Supernatants

Cell supernatant was clarified at 2,000×g during 10 min to remove cellular debris. Then it was aliquoted in 40 ml tubes and frozen at −80° C. Three tubes were thawed, and particles size distribution and concentration were determined by nanoparticle tracking analysis (NTA) using a ZetaView (PARTICLEMETRIX®, Germany) with a 405 nm laser. Before measurements, EVs were diluted 100 times with sterile PBS (confirmed to be particle-free by NTA measurement). For each sample, a sensitivity of 80 and a shutter of 100 were set.

### b) Further Characterization of EVs

EVs from cell supernatant were concentrated and purified by two consecutive runs of ultracentrifugation at 150,000×g during 90 min using an optima MAX-XP ultracentrifuge (BECKMAN COULTER®, UK). Concentrated EVs were analyzed by ExoView (NANOVIEW BIOSCIENCES®, USA). The sample was diluted at 1×10⁸ EV/ml in the kit's reagent A. The sample was incubated on the ExoView Tetraspanin Chip for human EVs placed in a 24-well plate for 16 h at room temperature. The chips were washed 3 times with reagent A. Chips were incubated with ExoView Tetraspanin Labelling antibodies that consist of anti-CD81 Alexa-555, anti-CD63 Alexa-488, and anti-CD9 Alexa-647. The antibodies were diluted 1:600 in immunofluorescence blocking solution. The chips were incubated with 250 μl of the labelling solution for 1 h, washed in solution A (PBS with 0.05% Tween-20), then in solution B (PBS alone) 3 times and dried. The chips were imaged with the ExoView R100 reader using the NScan acquisition software. The data were analyzed using ExoViewer.

Human HGF was specifically determined by the Human HGF Elisa Quantification Kit (THERMOFISHER SCIENTIFIC®) following the manufacturer's instructions.

### c) NanoLC-MS/MS Protein Identification and Quantification

S-Trap™ micro spin column (PROTIFI®, Hutington, USA) digestion was performed on 40 μg of cell lysate, supernatant and extra-vesicles according to manufacturer's instructions. Briefly, proteins were alkylated with 50 mM iodoacetamide in 5% SDS and 1.2% aqueous phosphoric acid. Colloidal protein particulate was formed with the addition of 6 times the sample volume of S-Trap binding buffer (90% aqueous methanol, 100 mM TEAB, pH 7.1). The protein mixtures were transferred into the S-Trap micro columns and centrifuged at 4,000×g for 30 seconds, and washed with 150 μL S-Trap binding buffer. Samples were then digested with 4 μg of trypsin (PROMEGA®) at 47° C. for 1 h. Peptides were eluted according to the manufacturer's protocol, and dried in Speed Vacuum.

They were resuspended in 10% ACN, 0.10% TFA in HPLC-grade water for LC-MS+MS analysis using the nanoRSLC-Q Exactive PLUS (RSLC Ultimate 3000 (THERMOFISHER SCIENTIFIC®, Waltham MA, USA). Peptides (1-2 μg) were loaded onto a p-precolumn (Acclaim PepMap 100 C18, cartridge, 300 μm i.d.×5 mm, 5 μm, THERMOFISHER SCIENTIFIC®), and were separated on a 50 cm reversed-phase liquid chromatographic column (0.075 mm ID, Acclaim PepMap 100, C18, 2 μm, THERMOFISHER SCIENTIFIC®) using mobile phase A (H₂O with 0.1% formic acid), and mobile phase B (80% acetonitrile, 0.08% formic acid). Peptides were eluted from the column with a gradient of 5% to 40% for 120 minutes, of 40% to 80% for 1 minute, and then the gradient stayed at 80% for 5 minutes after which it returned to 5% to re-equilibrate the column for 20 minutes before the next injection.

Eluted peptides were analyzed by data dependent MS/MS, using top-10 acquisition method and were fragmented by higher-energy collisional dissociation (HCD). MS scans and MS/MS scans were performed at a resolution of 70,000 and 17,5000 respectively. MS and MS/MS AGC target were set to 3×10⁶ and 1×10⁵ counts with maximum injection time set to 200 ms and 120 ms, respectively. The MS scan range was from 400 to 2,000 m/z. Dynamic exclusion was set at 30 seconds.

The MS files were processed with the Proteome Discoverer software version 2.4.0.305 and searched with Mascot search engine against the UniProtKB/Swiss-Prot Homo sapiens database (release 15 Apr. 2019, 20415 entries). To search parent mass and fragment ions, a mass deviation was set to 3 ppm and 20 ppm respectively. Other search parameters included: a minimum peptide length of 7 amino acids with a strict specificity for trypsin cleavage, carbamidomethylation (Cys) as fixed modification, whereas oxidation (Met) and N-term acetylation as variable modifications.

### 4.2 Results

a) Purification and Size Distribution of EVs Secreted by pStemHep in Cell Supernatants

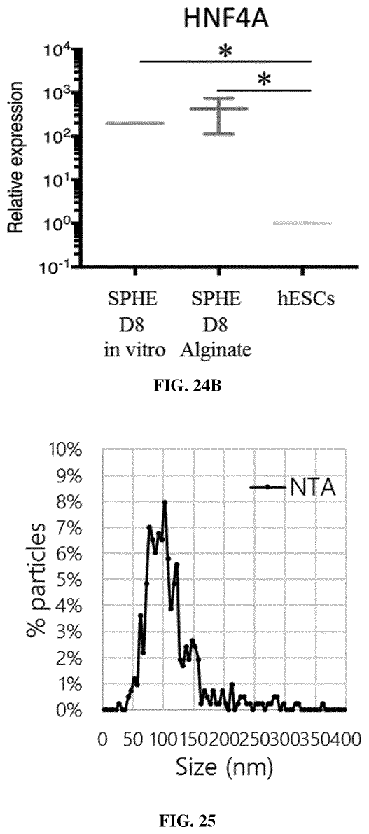

Cell supernatant was studied by NTA. A high number of particles of 4.2±0.4×10⁹ particles/mL was measured, which corresponds to a total particles of (2.0±0.2)×10¹² for 2×10⁹ seeded cells, meaning around 10³ particles/cells. Their size around 100 nm corresponds well to an EVs distribution (FIG. 25 and Table 4).

TABLE 4
EVs size distribution

| | Mean¹ |
|---|---|
| Diameter mode (nm) | 98.0 ± 5.5 |
| Diameter Mean (nm) | 103.9 ± 10.9 |
| Concentration (particle/mL) | 4.2 ± 0.4 E+09 |

¹diameter mean, mode, and concentration of particles present in supernatant samples, represented as the mean ± SD of three independent measurements by NTA.

### b) Further Characterization of the EVs

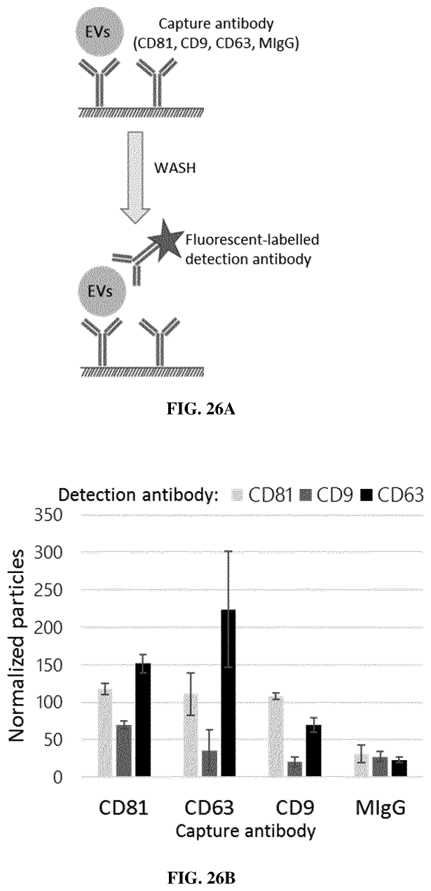

The obtained data indicated that EVs could be immuno-captured by the three tested anti-tetraspanins (CD63/CD81/CD9 antibodies; see FIG. 26A) with around 150 to 300 particles detected per spot. Most of the signal was associated with CD63 (40-70%) and to a lesser extent with CD81 markers (35-70%) (FIG. 26B). Respectively 2 and 16% of the immuno-captured EVs were expressing 3 or 2 of the 3 tetraspanin markers (FIG. 26C). This confirms the presence of EVs (tetraspanin positive) among the purified particles.

### c) NanoLC-MS/MS Protein Identification and Quantification

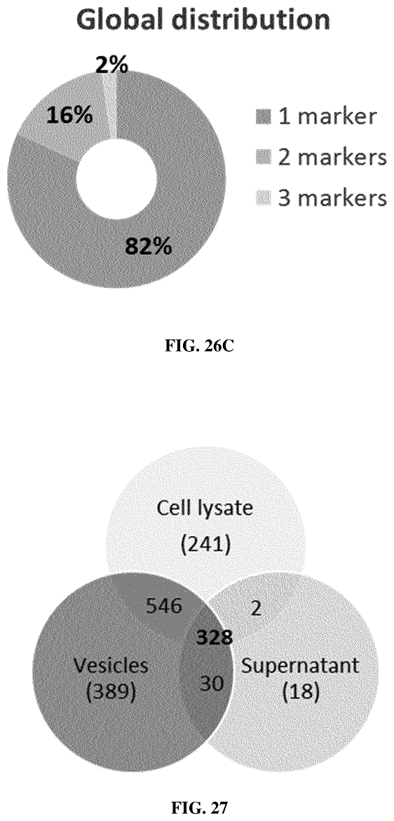

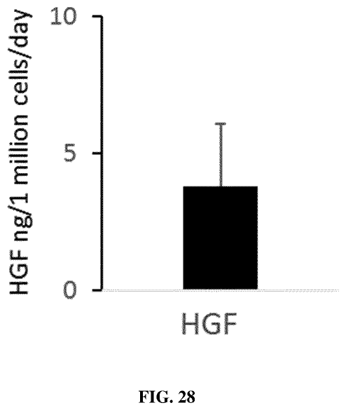

Proteomic analysis on the purified vesicles was compared with proteins from the cell lysate and of the whole supernatant. From the 0,5 μg of proteins analyzed, the vesicle sample showed the highest diversity in expressed proteins, with most of them shared with the cell lysate (FIG. 27). Many cytosolic and membrane proteins classically present in EVs were encountered in the vesicle sample at a high concentration. With this proteomic analysis, it was confirmed that there was significant EVs and exosomes in the supernatant of the cultured cells and that it was possible to purify them. Of note, EVs contains the same proteins of interest as the cells, such as AFP, HGF, apolipoteins (Tables 5 and 6, FIG. 28), as well as proteins that are usually found in EVs (Table 7).

TABLE 5
Proteins of interest that are found in the EVs

| Uniport | | Purified |
|---|---|---|
| accession | Protein description (Gene name) | vesicles |
| P02771 | Alpha-fetoprotein O (AFP) | 3 |
| P02647 | Apolipoprotein A-I (APOA1) | 20 |
| P04114 | Apolipoprotein B-100 (APOB) | 11 |
| P02649 | Apolipoprotein E (APOE) | 45 |
| P27487 | Dipeptidyl peptidase 4 (DPP4) | 49 |
| P17302 | Gap junction alpha-1 protein (GJA1) | 2 |
| P14210 | Hepatocyte growth factor (HGF) | 2 |
| P05783 | Keratin, type I cytoskeletal 18 (KRT18) | 23 |
| P08727 | Keratin, type I cytoskeletal 19 (KRT19) | 24 |
| P09486 | SPARC | 2 |
| P08670 | Vimentin O (VIM) | 42 |

TABLE 6
Other proteins of interest that are found in EVs

| Uniport | | Purified |
|---|---|---|
| accession | Protein description (Gene name) | vesicles |
| P07585 | Decorin (DCN) | 14 |
| P51654 | Glypican-3 (GPC3) | 6 |
| P17936 | Insulin-like growth factor-binding protein 3 (IGFBP3) | 11 |
| P23229 | Integrin alpha-6 (ITGA6) | 14 |
| P40189 | Interleukin-6 receptor subunit beta (IL6ST) | 5 |
| P13796 | Plastin-2 (LCP1) | 11 |

TABLE 7
Proteins that are usually found in EVs

| Uniprot | | Purified |
|---|---|---|
| accession | Protein description (Gene name) | vesicles |
| P60709 | Actin, cytoplasmic 1 (ACTB) ^{(1) (2)} | 99 |
| P63261 | Actin, cytoplasmic 2 (ACTG1) ^{(1) (2)} | 98 |
| P06733 | Alpha-enolase (ENO1) ⁽¹⁾ | 56 |
| P07355 | Annexin A2 (ANXA2) ^{(1) (2)} | 53 |
| P08758 | Annexin A5 (ANXA5) ^{(1) (2)} | 41 |
| P08133 | Annexin A6 (ANXA6) ⁽²⁾ | 75 |
| P98160 | Basement membrane-specific heparan sulfate | 52 |
| | proteoglycan core protein (HSPG2) ⁽³⁾ | |
| Q00610 | Clathrin heavy chain 1 (CLTC) ⁽²⁾ | 99 |
| P68104 | Elongation factor 1-alpha 1 (EEF1A1) ⁽¹⁾ | 40 |
| P13639 | Elongation factor 2 (EEF2) ⁽¹⁾ | 22 |
| P04406 | Glyceraldehyde-3-phosphate dehydrogenase | 68 |
| | (GAPDH) ⁽¹⁾ | |
| P11142 | Heat shock cognate 71 kDa protein (HSPA8) ^{(1) (2)} | 47 |
| P07900 | Heat shock protein HSP 90-alpha (HSP90AA1) ^{(1) (2)} | 43 |
| P08238 | Heat shock protein HSP 90-beta (HSP90AB1) ^{(1) (2)} | 54 |
| P14618 | Pyruvate kinase PKM (PKM) ⁽¹⁾ | 35 |
| P02786 | Transferrin receptor protein 1 (TFRC) ⁽³⁾ | 21 |
| Q71U36 | Tubulin alpha-1A chain (TUBA1A) ⁽²⁾ | 46 |
| P68363 | Tubulin alpha-1B chain (TUBA1B) ⁽²⁾ | 49 |
| P07437 | Tubulin beta chain (TUBB) ⁽²⁾ | 48 |
| Q13885 | Tubulin beta-2A chain (TUBB2A) ⁽²⁾ | 47 |
| Q9BVA1 | Tubulin beta-2B chain (TUBB2B) ⁽²⁾ | 47 |
| Q13509 | Tubulin beta-3 chain (TUBB3) ⁽²⁾ | 27 |
| P04350 | Tubulin beta-4A chain (TUBB4A) ⁽²⁾ | 36 |
| P68371 | Tubulin beta-4B chain (TUBB4B) ⁽²⁾ | 45 |
| Q9BUF5 | Tubulin beta-6 chain (TUBB6) ⁽²⁾ | 27 |

⁽¹⁾ Proteins from the top twenty highest occurring proteins found in studies on EVs (ExoCarta database),
⁽²⁾ Cytosolic proteins recovered in EVs (MISEC 2018 classification);
⁽³⁾ the transmembrane or GPI-anchored proteins associated to plasma membrane and/or endosomes.

### REFERENCES

Arroyo V, Moreau R, Kamath P S, Jalan R, Ginés P, Nevens F, Fernandez J, To U, Garcia-Tsao G, Schnabl B. Acute-on-chronic liver failure in cirrhosis. Nat Rev Dis Primers. 2016 Jun. 9(2):16041.
Cameron K, Tan R, Schmidt-Heck W, Campos G, Lyall M J, Wang Y, Lucendo-Villarin B, Szkolnicka D, Bates N, Kimber S J, Hengstler J G, Godoy P, Forbes S J, Hay D C. Recombinant Laminins Drive the Differentiation and Self-Organization of hESC-Derived Hepatocytes. Stem Cell Reports. 2015 Dec. 8; 5(6):1250-1262.
Carpenter S P, Lasker J M, Raucy J L. Expression, induction, and catalytic activity of the ethanol-inducible cytochrome P450 (CYP2E1) in human fetal liver and hepatocytes. Mol Pharmacol. 1996; 49(2):260-268.
Chen S, Wang J, Ren H, Liu Y, Xiang C, Li C, Lu S, Shi Y, Deng H, Shi X. Hepatic spheroids derived from human induced pluripotent stem cells in bio-artificial liver rescue porcine acute liver failure. Cell Research (2019) 0:1-3; https://doi.org/10.1038/s41422-019-0261-5.
Chinnici C M, Timoneri F, Amico G, Pietrosi G, Vizzini G, Spada M, Pagano D, Gridelli B, Conaldi P G. Characterization of Liver-Specific Functions of Human Fetal Hepatocytes in Culture. Cell Transplant. 2015; 24(6):1139-1153.
Chung Y, Klimanskaya I, Becker S, Li T, Maserati M, Lu S J, Zdravkovic T, Ilic D, Genbacev O, Fisher S, Krtolica A, Lanza R. Human embryonic stem cell lines generated without embryo destruction. Cell Stem Cell. 2008 Feb. 7; 2(2):113-7.
De Coppi P1, Bartsch G Jr, Siddiqui M M, Xu T, Santos C C, Perin L, Mostoslavsky G, Serre A C, Snyder E Y, Yoo J J, Furth M E, Soker S, Atala A. Isolation of amniotic stem cell lines with potential for therapy. Nat Biotechnol. 2007 January; 25(1):100-6.
Dellavalle A1, Sampaolesi M, Tonlorenzi R, Tagliafico E, Sacchetti B, Perani L, Innocenzi A, Galvez B G, Messina G, Morosetti R, Li S, Belicchi M, Peretti G, Chamberlain J S, Wright W E, Torrente Y, Ferrari S, Bianco P, Cossu G. Pericytes of human skeletal muscle are myogenic precursors distinct from satellite cells. Nat Cell Biol. 2007 March; 9(3):255-67.
Dhawan A, Strom S C, Sokal E, Fox I J. Human hepatocyte transplantation. Methods Mol Biol. 2010; 640:525-34.
Dhawan A, Chaijitraruch N, Fitzpatrick E, Bansal S, Filippi C, Lehec S C, Heaton N D, Kane P, Verma A, Hughes R D, Mitry R R. Alginate microencapsulated human hepatocytes for the treatment of acute liver failure in children. J Hepatol. 2020 May; 72(5):877-884.
D'Ippolito G, Diabira S, Howard G A, Menei P, Roos B A, Schiller P C. Marrow-isolated adult multilineage inducible (MIAMI) cells, a unique population of postnatal young and old human cells with extensive expansion and differentiation potential. J Cell Sci. 2004 Jun. 15; 117(Pt 14):2971-81.
Glorioso J M, Mao S A, Rodysill B, Mounajjed T, Kremers W K, Elgilani F, Hickey R D, Haugaa H, Rose C F, Amiot B, Nyberg S L. Pivotal preclinical trial of the spheroid reservoir bioartificial liver. J Hepatol. 2015 August; 63(2):388-398.
Gonzalez F, Boué S, Izpisúa Belmonte J C. Methods for making induced pluripotent stem cells: reprogramming á la carte. Nat Rev Genet. 2011 April; 12(4):231-42.
Hansel M C, Gramignoli R, Skvorak K J, Dorko K, Marongiu F, Blake W, Davila J, Strom S C. The history and use of human hepatocytes for the treatment of liver diseases: the first 100 patients. Curr Protoc Toxicol. 2014 Nov. 6; 62:14.12.1-23. Parallel derivation of isogenic human primed and naive induced pluripotent stem cells.
Jochheim A, Hillemann T, Kania G, Scharf J, Attaran M, Manns M P, Wobus A M, Ott M. Quantitative gene expression profiling reveals a fetal hepatic phenotype of murine ES-derived hepatocytes. Int J Dev Biol. 2004 February; 48(1):23-9.
Kilens S, Meistermann D, Moreno D, Chariau C, Gaignerie A, Reignier A, Leliévre Y, Casanova M, Vallot C, Nedellec S, Flippe L, Firmin J, Song J, Charpentier E, Lammers J, Donnart A, Marec N, Deb W, Bihouée A, Le Caignec C, Pecqueur C, Redon R, Barriere P, Bourdon J, Pasque V, Soumillon M, Mikkelsen T S, Rougeulle C, Freour T, David L; Milieu Interieur Consortium. Nat Commun. 2018 Jan. 24; 9(1):360. doi: 10.1038/s41467-017-02107-w.).
Kögler G, Sensken S, Airey J A, Trapp T, Miischen M, Feldhahn N, Liedtke S, Sorg R V, Fischer J, Rosenbaum C, Greschat S, Knipper A, Bender J, Degistirici O, Gao J, Caplan A I, Colletti E J, Almeida-Porada G, Müller HW, Zanjani E, Wernet P. A new human somatic stem cell from placental cord blood with intrinsic pluripotent differentiation potential. J Exp Med. 2004 Jul. 19; 200(2):123-35.
Larsen F S, Schmidt L E, Bernsmeier C, Rasmussen A, Isoniemi H, Patel V C, Triantafyllou E, Bernal W, Auzinger G, Shawcross D, Eefsen M, Bjerring P N, Clemmesen J O, Hockerstedt K, Frederiksen H J, Hansen B A, Antoniades C G, Wendon J. High-volume plasma exchange in patients with acute liver failure: An open randomised controlled trial. J Hepatol. 2016; 64(1):69-78.
Miyazono K, Kusanagi K, Inoue H. Divergence and convergence of TGF-beta/BMP signaling. J Cell Physiol. 2001 June; 187(3):265-76. Review. Narayanaswamy R. and Torchilin V P. Hydrogels and their applications in targeted drug delivery. Molecules 2019, 24, 603.
Ntai A, La Spada A, De Blasio P, Biunno I. Trehalose to cryopreserve human pluripotent stem cells. Stem Cell Res. 2018; 31:102-112.
Pareja E, Gomez-Lechón MJ, Tolosa L. Alternative Cell Sources to Adult Hepatocytes for Hepatic Cell Therapy. Methods Mol Biol. 2017; 1506:17-42.
Payne C M, Samuel K, Pryde A, King J, Brownstein D, Schrader J, Medine C N, Forbes S J, Iredale J P, Newsome P N, Hay D C. Persistence of functional hepatocyte-like cells in immune-compromised mice. Liver Int. 2011 February; 31(2):254-62.
Peppas N A, Bures P, Leobandung W, Ichikawa H. Hydrogels in pharmaceutical formulations. Eur J Pharm Biopharm. 2000 July; 50(1):27-46.
Perez-Luna V H, Gonzalez-Reynoso O. Encapsulation of Biological Agents in Hydrogels for Therapeutic Applications. Gels. 2018 Jul. 11; 4(3). pii: E61. doi: 10.3390/gels4030061.
Pietrosi G, Vizzini G, Gerlach J, Chinnici C, Luca A, Amico G, D'Amato M, Conaldi P G, Li Petri S, Spada M, Tuzzolino F, Alio L, Schmelzer E, Gridelli B. Phases I-II Matched Case-Control Study of Human Fetal Liver Cell Transplantation for Treatment of Chronic Liver Disease. Cell Transplant. 2015; 24(8):1627-38.
Rao M S, Khan A A, Parveen N, Habeeb M A, Habibullah C M, Pande G. Characterization of hepatic progenitors from human fetal liver during second trimester. World J Gastroenterol. 2008 Oct. 7; 14(37):5730-7.
Roelandt P, Pauwelyn K A, Sancho-Bru P, Subramanian K, Bose B, Ordovas L, Vanuytsel K, Geraerts M, Firpo M, De Vos R, Fevery J, Nevens F, Hu W-S, Verfaillie C M. Human Embryonic and Rat Adult Stem Cells with Primitive Endoderm-Like Phenotype Can Be Fated to Definitive Endoderm, and Finally Hepatocyte-Like Cells. PLoS One. 2010; 5(8): e12101.
Sagi I, Chia G, Golan-Lev T, Peretz M, Weissbein U, Sui L, Sauer M V, Yanuka O, Egli D, Benvenisty N. Derivation and differentiation of haploid human embryonic stem cells. Nature. 2016 Apr. 7; 532(7597):107-11.
Sampaolesi M, Blot S, D'Antona G, Granger N, Tonlorenzi R, Innocenzi A, Mognol P, Thibaud J L, Galvez B G, Barthelemy I, Perani L, Mantero S, Guttinger M, Pansarasa O, Rinaldi C, Cusella De Angelis M G, Torrente Y, Bordignon C, Bottinelli R, Cossu G. Mesoangioblast stem cells ameliorate muscle function in dystrophic dogs. Nature. 2006 Nov. 30; 444(7119):574-9.
Siller R, Greenhough S, Naumovska E, Sullivan G J. Small-molecule-driven hepatocyte differentiation of human pluripotent stem cells. Stem Cell Reports. 2015 May 12; 4(5):939-52.
Struecker B, Raschzok N, Sauer I M. Liver support strategies: cutting-edge technologies. Nat Rev Gastroenterol Hepatol. 2014 March; 11(3):166-76.
Takayama K, Nagamoto Y, Mimura N, Tashiro K, Sakurai F, Tachibana M, Hayakawa T, Kawabata K, Mizuguchi H. Long-term self-renewal of human ES/iPS-derived hepatoblast-like cells on human laminin 111-coated dishes. Stem Cell Reports. 2013 Oct. 3; 1(4):322-35.
Takayama K, Akita N, Mimura N, Akahira R, Taniguchi Y, Ikeda M, Sakurai F, Ohara O, Morio T, Sekiguchi K, Mizuguchi H. Generation of safe and therapeutically effective human induced pluripotent stem cell-derived hepatocyte-like cells for regenerative medicine. Hepatol Commun. 2017 Oct. 12; 1(10):1058-1069.
Terry C, Dhawan A, Mitry R R, Lehec S C, Hughes R D. Optimization of the cryopreservation and thawing protocol for human hepatocytes for use in cell transplantation. Liver Transpl. 2010 February; 16(2):229-37.
Tolosa L, Caron J, Hannoun Z, Antoni M, López S, Burks D, Castell J V, Weber A, Gomez-Lechon M J, Dubart-Kupperschmitt A. Transplantation of hESC-derived hepatocytes protects mice from liver injury. Stem Cell Res Ther. 2015 Dec. 12; 6:246.
Warren L, Manos P D, Ahfeldt T, Loh Y H, Li H, Lau F, Ebina W, Mandal P K, Smith Z D, Meissner A, Daley G Q, Brack A S, Collins J J, Cowan C, Schlaeger T M, Rossi D J. Highly efficient reprogramming to pluripotency and directed differentiation of human cells with synthetic modified mRNA. Cell Stem Cell. 2010 Nov. 5; 7(5):618-30.
Zheng J F, Liang L J, Wu C X, Chen J S, Zhang Z S. Transplantation of fetal liver epithelial progenitor cells ameliorates experimental liver fibrosis in mice. World J Gastroenterol. 2006 Dec. 7; 12(45):7292-8.

1. An isolated population of cells, comprising at least 5% of hepatic stem-like cells expressing the alpha-fœtoprotein marker (AFP+) and not expressing the albumin marker (ALB−), or an extract thereof.

2. The isolated population of cells according to claim 1, wherein the hepatic stem-like cells are further expressing the T-Box Transcription Factor 3 marker (TBX3+) and/or the Hepatocyte Nuclear Factor 4 Alpha marker (HNF4A+), preferably the T-Box Transcription Factor 3 marker (TBX3+) and the Hepatocyte Nuclear Factor 4 Alpha marker (HNF4A+).

3. The isolated population of cells according to claim 1, wherein the hepatic stem-like cells are cryopreserved.

4. A particle, in particular a spheroid, comprising the isolated population of cells, or an extract thereof, according to claim 1.

5. A suspension comprising the isolated population of cells, or an extract thereof, according to claim 1.

6. A pharmaceutical composition comprising (i) the isolated population of cells, or an extract thereof, according to claim 1, and (ii) a pharmaceutically acceptable vehicle.

7. A medical device comprising the isolated population of cells, or an extract thereof according to claim 1.

8. A non-human animal model comprising the population of cells, or an extract thereof, according to claim 1, wherein the population of cells are heterologous.

9. (canceled)

10. A method of preventing and/or treating a fulminant liver disorder in a subject in need thereof comprising administering the population of cells, or an extract thereof, according to claim 1.

11. The method according to claim 10, wherein the fulminant liver disorder is an acute liver failure (ALF) or an acute chronic liver failure (ACLF).

12. The method according to claim 11, wherein the ACLF is associated with a liver disease selected in the group consisting of the non-alcoholic steatohepatitis (NASH); alcoholic hepatitis; viral-induced hepatitis; a cryptogenic liver disease; a malignant liver disease, such as hepatocellular carcinoma and cholangiocarcinoma; autoimmune hepatitis, a vascular liver disease, such as Budd-Chiari syndrome; a cholestatic liver disease; and an inherited metabolic liver disease, such as, Wilson's disease and an urea cycle disorder.

13. The population of cells, or an extract thereof, according to claim 1, wherein the population of cells is cryopreserved.

14. An in vitro method for screening a drug, said method comprising the steps of:
a. providing population of cells according to claim 1;
b. contacting said population of cells or extract thereof, from step (a), with a drug candidate;
c. measuring one or more biological parameter(s) and optionally comparing said one or more biological parameter(s) with one or more reference parameter(s);
d. determining whether the drug candidate is of therapeutic and/or diagnostic interest.

15. A kit for treating and/or preventing a fulminant liver disorder, said kit comprising:
a. a population of cells, or an extract thereof according to claim 1; and
b. a mean to administer said cells or extract thereof, population or extract thereof, or particle, or suspension or pharmaceutical composition.
